(19) United States
(12) Patent Application Publication
(10) Pub. No.: US 2024/0238243 A1
(43) Pub. Date: Jul. 18, 2024

(54) METHODS AND COMPOSITIONS FOR TREATMENT OF HEART FAILURE WITH PRESERVED EJECTION FRACTION

(71) Applicant: Northwestern University, Evanston, IL (US)

(72) Inventors: Hossein Ardehali, Evanston, IL (US); Yuki Tatekoshi, Evanston, IL (US)

(21) Appl. No.: 18/412,362

(22) Filed: Jan. 12, 2024

Related U.S. Application Data
(60) Provisional application No. 63/479,955, filed on Jan. 13, 2023.

(51) Int. Cl. A61K 31/382; A61P 9/04

(52) U.S. Cl. CPC A61K 31/382; A61P 9/04

(57) ABSTRACT

Disclosed are methods and compositions for treating or preventing heart failure with preserved ejection fraction that involve administering an O-linked N-acetylglucosamine transferase (OGT) inhibitor, for example 5S-GlcNHex, or increasing O-linked N-acetylglucosaminase (OGA) expression in endothelial cells in the heart.

### CROSS REFERENCE TO RELATED APPLICATIONS

This application claims the benefit of U.S. Provisional Application Ser. No. 63/479,955, filed Jan. 13, 2023, the entire contents of which are incorporated by reference herein.

### STATEMENT REGARDING FEDERALLY SPONSORED RESEARCH OR DEVELOPMENT

This invention was made with government supporn under grant numbers HL155953, HL138982, HL140927, and HL140973 awarded by the National Institutes of Health. The government has certain rights in the invention.

### BACKGROUND

Heart failure (HF) with preserved ejection fraction (HFpEF) is associated with heightened all-cause mortality, however, the precise molecular mechanisms driving the HFpEF pathogenesis remain poorly understood. HFpEF is more prevalent with older age, and frequently coexists with multiple comorbidities, such as hypertension, renal dysfunction, and diabetes. There are few treatment options for HFpEF, and the current therapies primarily address symptom relief and target comorbid conditions. Evidence suggests that endothelial cell (EC) dysfunction plays a critical role in the development of HFpEF, including microvascular dysfunction (MVD). MVD is associated with worse diastolic dysfunction that increases in the major adverse cardiovascular events. A positive correlation between EC dysfunction and exercise intolerance in HFpEF patients is also reported. However, the mechanism underlying the observed EC dysfunction and the causal relationship between EC dysfunction and HFpEF development remain elusive. Notably, two major risk factors for the development of HFpEF, diabetes and hypertension, are both associated with increased protein O-linked N-acetylglucosaminylation (O-GlcNAcylation) in ECs. Consequently, it is conceivable that HFpEF pathophysiology may involve increased protein O-GlcNAcylation in ECs as a consequence of predisposing risk factors.

ECs highly rely on glycolysis to produce energy and perform their physiological functions, as they generate >85% of their ATP through glycolysis even in oxygen-replete conditions. Hexokinases (HKs) carry out the first step in glucose utilization by phosphorylating glucose to glucose-6-phosphate (G6P), thereby committing it to various downstream metabolic pathways. While glycolysis yields energy-producing intermediates, it can also be shunted into other pathways, including the pentose phosphate pathway (PPP), hexosamine biosynthesis pathway (HBP), or be converted to glucose-1-phosphate for glycogen synthesis.

Identifying molecular mechanisms underlying the development of HFpEF and compounds targeting the pathway to treat or prevent this devastating disease is of great interest. Here the inventors demonstrate a significant role for HK1 mitochondrial localization in the fate of HBP intermediates into either N-glycosylation or O-GlcNAcylation machinery in ECs. They demonstrate that HK1 is predominantly expressed in ECs relative to other cardiac cell types and dislocates from mitochondria in the setting of HFpEF. The findings highlight the significant role of HK1 mitochondrial binding in orchestrating HBP metabolites to be shuttled among protein glycosylation machineries, providing a promising therapeutic pathway for HFpEF by targeting the elevation of protein O-GlcNAcylation.

### SUMMARY

Disclosed are methods and compositions for treating or preventing heart failure with preserved ejection fraction (HFpEF). The disclosed methods include treating or preventing heart failure with preserved ejection fraction comprising administering a composition comprising an 0-linked N-acetylglucosamine transferase (OGT) inhibitor to treat or prevent HFpEF. In the methods, the OGT inhibitor reduces protein O-linked N-acetylglucosaminylation (0-GlcNAcylation) in endothelial cells of the heart. An example of an OGT used in the methods is 2-deoxy-2-N-hexanamide-5-thio-D-glucopyranoside (5SGlcNHex). The methods may include administering a pharmaceutical composition comprising the OGT inhibitor to treat I-FpEF, such as to a subject who has experienced HFpEF.

The disclosed methods also include treating or preventing HFpEF comprising administering a composition that increases O-linked N-acetylglucosaminase (OGA) in endothelial cells of the heart. In embodiments, the composition is administered to a subject who has experienced or is at risk of experiencing HFpEF, and preferably the method protects against developing HFpEF or treats HFpEF. In some embodiments, the composition protects against angiogenic defect in the endothelial cells, restores or maintains diastolic function, and/or reduces or eliminates cardiac hypertrophy and microvascular rarefaction in a heart following HFpEF.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-IF show HK1 is highly expressed in endothelial cells (ECs) compared to cardiomyocytes (CMs) and fibroblasts (FBs). (FIG. 1A) Violin plot of mRNA expression levels of glycolytic enzymes in CMs. FBs, and ECs from human heart single-cell RNA-seq data (n=15 in CMs, n=19 in FBs and n=4 in ECs). (FIG. 1B) Representative images of isolated CMs, FBs and ECs. (FIG. 1C) mRNA expression levels of genes specific to CMs, FBs and ECs isolated from mouse hearts. Data were analyzed by one-way ANOVA with Tukey's test for multiple group comparison. (FIG. 1D) Representative immunoblots of isolated cells from mouse hearts, human primary FBs (hFB), and HUVECs. (FIG. 1E) Summary of densitometry of immunoblot analysis from FIG. 1D (n=2-3). (FIG. 1F) Representative immunofluorescent image of hearts from C57BL/6J mice probed with hexokinase-1 (HK1) (red), CD31 (white), and 4′,6-diamnidino-2-phenylindole (DAPI) (blue). The scale bar indicates 10 m. Data were analyzed by one-way ANOVA with Tukey's test for multiple group comparison (FIG. 1E).

FIGS. 2A-2O show HK1 is dislocated from mitochondria in ECs from HFpEF mice. (FIG. 2A) Representative images of pulsed-wave Doppler of trans-mitral flow (TMF) in mice treated with normal chow (NC) or high fat diet (HFD) HFD_L-NAME. The vertical scale bar indicates 200 mm/s, and the horizontal one indicates 0.05 sec. Bar graphs indicate the summary of E/A and E/e' (n=4-5). (FIG. 2B) Summary of the left atrial (LA) area (n=4-5). (FIG. 2C) Representative echocardiography images of M-mode of LVs in the mice. The vertical scale bar indicates 1 mm, and the horizontal one indicates 0.1 sec. (FIG. 2D) Summary of EF, FS, IVSd, LVDd and LVDs (n 4-5 mice per group). (FIG. 2E) Running distance during exercise exhaustion test (n=4-5). (FIG. 2F) Systolic, mean, and diastolic blood pressure (BP) (sBP, mBP, and dBP) (n=4). (FIG. 2G) Representative immunofluorescent images of hearts probed with CD31 (white) and DAPI (blue). The scale bar indicates 25 m. The bar graph indicates microvascular density (n=8-10 areas per group). Data were analyzed by unpaired Student t test. (FIG. 2H) Representative immunoblots of protein samples from isolated ECs from the heart. (FIG. 2I) Summary bar graph of densitometry of FIG. 2H (n=2-3). (FIG. 2J) Representative immunofluorescent images of freshly isolated ECs from hearts of mice treated with NC or HFD_L-NAME probed with HK1 (green), ATP synthase beta (red), and DAPI (blue). The scale bar indicates 2.5 m. (FIG. 2K) Histogram of relative intensity of HK1 and ATP synthase-0 on the white lines on the images in FIG. 2J. (FIG. 2L) Pearson's R values of localization of HK1 and ATP synthase-0 from the fluorescent images (n=15 cells per group). (FIG. 2M) Representative images of immunogold electron microscopy with anti-HK1 antibody. White arrows indicate the Z-line of myofibrils in cardiomyocytes. Yellow arrows indicate the structure of mitochondria. Red arrows indicate the presence of gold particles on OMM in both groups. (FIG. 2N) Representative images of IEM with anti-HK1 antibody in heart sections of mice treated with NC or HFD_L-NAME. (FIG. 2O) Number of gold particles not on the mitochondria (i.e., in the cytoplasm and nucleus) (n=22 areas per group). Data were analyzed by unpaired Student t test (FIGS. 2A, 2I, and 2L) or Mann-Whitney test for (FIG. 2O).

FIGS. 3A-3CC illustrate that AE1HK1 mice develop HFpEF at 40 weeks of age. (FIG. 3A) Representative immunoblots of cytosolic and mitochondrial protein fractions from hearts of WT and AE1HK1 mice. (FIG. 3B) Representative images of M-mode of LVs at the age of 20 and 40 weeks. The vertical scale bar indicates 2 mm, and the horizontal one indicates 0.1 sec. The bar graphs indicate the summary of EF, the intraventricular wall thickness in diastole (IVSTd or IVSd), LVDd, and LVDs (n=8-9 mice per group). (FIG. 3C) Representative images of pulsed-wave Doppler of TMF and bar graph of E/A (n=8-9 mice per group) The vertical scale bar indicates 400 mm/s, and the horizontal one indicates 0.1 sec. (FIG. 3D) Representative images of tissue Doppler and the bar graph of e' and E/e' (n=8-9 mice per group). The vertical scale bar indicates 30 mm/s, and the horizontal one indicates 0.1 sec. (FIG. 3E) LA area of the hearts of WT and AE1HK1 mice (n=8-9 mice per group). (FIG. 3F) Running distance during exercise exhaustion test (n=7-8 mice per group). (FIG. 3G). BP in WT and AE1HK1 mice. The bar graphs indicate sBP, mBP, and dBP of male and female mice at the ages of 12, 24, and 40 weeks (n=5 mice per group). Data were analyzed by two-way ANOVA with Tukey's test for multiple group comparison. (FIG. 3H) Fluid volume in lungs calculated by subtracting dry lung weight from wet one at 40 weeks of age (n=8 mice per group). (FIG. 3I) Representative images of heart sections with MT staining. The scale bar indicates 20 m. The bar graph indicates % fibrosis area in the heart sections (n=9-10 fields per group). (FIGS. 3J-3K) Echocardiography in WT and AE1HK1 female. The bar graphs indicate summary of EF, FS, IVSd, PWTd, LVDd, LVDs, E/A, e', E/e', LA area, and exercise tolerance (n 6-9 mice per group). Data were analyzed by two-way ANOVA with Tukey's test for multiple group comparison for EF, FS, IVSd, PWTd, LVDd, LVDs, E/A and e' or by Student t test for LA area and Exercise tolerance. (FIG. 3L) Representative images ofM-mode of LVs in the mice treated with HFD for 20 weeks. The vertical scale bar indicates 2 mm, and the horizontal one indicates 0.1 sec. The bar graphs indicate the sumimary ofEF, IVSd, LVDd, and LVDs (n=8-9 mice per group). (FIG. 3M) Representative tissue doppler imaging (TDI) and the bar graphs of e' and E/e' (n=8-9 mice per group). The vertical scale bar indicates 30 mm/s, and the horizontal one indicates 0.1 sec. (FIG. 3N) Representative images of pulsed-wave Doppler of TMF and the bar graph of E/A. The vertical scale bar indicates 400 mm/s, and the horizontal one indicates 0.1 sec. (n=8-9 mice per group). (FIG. 3O) Running distance during exercise exhaustion test (n=7-8 mice per group). (FIG. 3P) Summary of body weight (BW) (n=8-9) and (FIG. 3Q) sBP, mBP and dBP (n=7-8 mice per group) in WT and AElHK1 mice. Data were analyzed by two-way ANOVA with Tukey's test for multiple group comparison (FIGS. 3L-3O) and unpaired Student t test (FIGS. 3P, 3Q). AE1 refers to AE1HK1 mice. FIGS. 3R-3W show physiological data of several organs in ΔE1HK1 mice at 40 weeks of age: (FIG. 3R) Serum ALT level, (FIG. 3S) serum Cr level, (FIG. 3T) serum BUN level, (FIG. 3U) Kidney weight divided by BW, (FIG. 3V) Grip strength divided by BW and (FIG. 3W) Gastrocnemius muscle weight divided by BW of WT and AE1HK1 mice. (n=4) Data were analyzed by unpaired Student/test. (FIG. 3X) Ca2+ half decay time in isolated CMs from 20-week-old mice (n=5 cells per group). (FIG. 3Y) Representative immunofluorescent images of hearts probed with CD31 (white) and DAPI (blue). The scale bar indicates 25 m. The bar graph indicates microvascular density in the heart (n=9 fields per group). (FIG. 3Z) Representative images of pulse doppler images of left coronary artery blood flow rate. The vertical scale bar indicates 400 mm/s, and the horizontal one indicates 0.1 sec. The bar graph indicates a summary of CFR calculated by maximus flow rate in 2.5% isoflurane divided by the one in 1% isoflurane. (FIG. 3AA) Time course of EF, LVDd, PWTd, E/A, e' and E/e' in C57BL/6J after HFD_L-NAME treatment (n=7-10). (FIG. 3BB) Representative immunofluorescent images of hearts of mice treated with HFD_L-NAME probed with CD31 (white) and DAPI (blue). The scale bar indicates 50 m. The bar graph indicates microvascular density (n=8 areas per group). (FIG. 3CC) Representative images of pulse doppler images of left coronary artery blood flow rate. The bar graph indicates a summary of calculated CFR (n=4). Data were analyzed by two-way ANOVA with Tukey's test for multiple group comparison (FIGS. 3B, 3C, 3D and 3I) or unpaired Student t test (FIGS. 3E, 3F, 3H, 3X, 3Y, 3Z, 3AA, 3BB, and 3CC).

FIGS. 4A-4H show that isolated ECs from hearts of AE1HK1 mice display reduced angiogenic potential. (FIG. 4A) Representative immunoblots of protein samples of isolated ECs from the heart of WT and ΔE1HK1 mice. The bar graph indicates a summary of densitometry (n=4). (FIG. 4B) Representative immunofluorescent images of isolated ECs from hearts probed with HK1 (green), ATP synthase-0 (red), and DAPI (blue). The scale bar indicates 25 km. (FIG. 4C) Pearson's R values of localization of HK1 and ATP synthase-0 from the fluorescent images (n=10 cells per group). (FIG. 4D) Representative images of tube formation assay in isolated ECs from the heart of WT and AE1HK1 mice. The scale bar indicates 250 km. The bar graphs indicate the number of junctions, meshes, segments, and total length of branching (n=6 fields per group). (FIG. 4E) Representative immunofluorescent images of isolated ECs probed with BrdU (green) and DAPI (blue). The scale bar indicates 50 m. The bar graph indicates % BrdU per DAPI positive cells (n=7 fields per group). (FIG. 4F) Representative images of cells migrating pores on the membrane stained by crystal violet. The scale bar indicates 250 m. The bar graph indicates the number of cells per field (n=10 fields per group). (FIG. 4G) MMP is calculated by the relative intensity of fluorescence of TMRE and mitotracker green (n=8 cells per group). (FIG. 4H) Representative images of the vessel sprouting in retinas from WT and AE1HK1 neonates. The scale bar indicates 500 m. The graph shows the summary of the sprouting front radius from the center. Data were analyzed by unpaired Student t test (FIGS. 4A, 4C, 4D, 4E, 4F and 4G) or two-way ANOVA with Bonferroni's test for multiple group comparison (FIG. 4H).

FIGS. 5A-5P show that levels of metabolites in HBP are reduced in isolated ECs from the hearts of AE1HK1 mice. (FIG. 5A) Levels of ATP in isolated ECs from the heart of WT and ΔE1HK1 mice. (FIG. 5B) Energy charge calculated by ([ATP]+½[ADP]/[ATP]+[ADP]+[AMP]) in isolated ECs. (FIG. 5C) Representative immunoblots of protein samples of isolated ECs. The bar graph indicates relative densities of P-TI 72-AMPK/total AMPKa (n=4). (FIG. 5D) ECAR before the administration of oligomycin (basal ECAR) and the one displaying the highest ECAR (maximal ECAR) (n=8-9 per group). (FIG. 5E) Maximal mitochondrial respiration calculated by basal OCR subtracted from maximal OCR (n=8-9 per group). (FIG. 5F) Heatmap plots of relative levels of metabolites in glycolysis and branched metabolic pathway from glycolysis. (FIG. 5G) Schematic of HBP and subsequent glycosylation machinery. (FIG. 5H) Levels of metabolites in HBP in isolated EBCs from the heart ofWT and ΔE1HK1 mice (n=4 per group). (FIG. 5I) The proportion of metabolites with isotope-labeled carbon in HBP in isolated ECs treated with U-¹C6 glucose for 30 minutes or 2 hours. (n=4 per group). (FIG. 5J-5N) Isotope tracing metabolomics using U-¹³C6 glucose in isolated ECs from the hearts of WT and AE1HK1 mice. Bar graphs indicate the proportion of metabolites with isotope-labeled carbon in isolated ECs from the heart of mice treated with U-³C glucose for 30 minutes or 2 hours (n=4 per group). (FIG. 50) Representative immunoblots of protein samples of isolated ECs. The bar graph indicates a summary of densitometry (n=4). (FIG. 5P) Relative mRNA expression levels of genes related to HBP in isolated ECs from the heart. Data were analyzed by unpaired Student t test.

FIGS. 6A-6P show that dislocation of HK1 in ECs causes reduced protein N-glycosylation. (FIG. 6A) Representative lectin-blot with ConA-HRP, immunoblot of GAPDH, and ponceau S staining of protein samples of freshly isolated ECs from the heart of WT and AE1HK1 mice. The bar graph indicates a summary of densitometry (n=3). (FIG. 6B) Representative immunofluorescent images of hearts probed with WGA (red). The scale bar indicates 25 m. The bar graph indicates the intensity of WGA divided by the perimeter of capillaries (n=367-526 capillaries per group). (FIG. 6C) Relative mRNA expression levels of genes related to UPR in isolated ECs (n=3). (FIG. 6D) Representative lectin-blot with ConA-HRP and immunoblot of GAPDH in protein samples of HUVEC overexpressing eGFP, FL-HK1, or Tr-HK1 conjugated with eGFP. (FIG. 6E) Table of selected proteins identified by mass spectrometry in protein samples IPed by an anti-HK1 antibody in HepG2 cells overexpressing eGFP, FL-HK1, and Tr-HK1 conjugated with eGFP. The numbers in the table indicate the number of peptides detected corresponding to the part of the amino acid sequence of the proteins listed in the table. (FIG. 6F) Representative immunoblots of protein samples of isolated ECs from the heart. The bar graph indicates a summary of densitometry (n=3). Data were analyzed by unpaired Student t test (FIGS. 6A, 6B, 6C and 6F). FIGS. 6G-6H show characterization of HUVECs stably overexpressing eGFP, FL-HK1, and Tr-HK1. (FIG. 6G) Representative immunoblot images of protein samples from HUVEC stably overexpressing eGFP, FL-HK1, and Tr-HIK1 with anti-eGFP, HK1, and GAPDH antibodies. Black arrows indicate the position of proteins suggested in the figure. (FIG. 6H) Representative immunofluorescent images of HUVEC stably overexpressing eGFP, FL-HK1 and Tr-HK1 probed with HK1 (green), ATP synthase-β (red), and DAPI (blue). The scale bar indicates 25 m. FIGS. 61-6J are representative images of Ped protein samples of HUVEC overexpressing eGFP, FL-HK1, and Tr-HK1, or of heart lysates from WT and ΔE1HK1. (FIG. 6I) Representative images of immunoprecipitated protein samples of HUVEC overexpressing eGFP, FL-HK1, and Tr-HK1 with anti-eGFP antibody. Black arrows indicate the position of proteins suggested in the figure. (FIG. 6J) Representative images of immunoprecipitated protein samples of heart lysates from WT and ΔE1HK1 with anti-HK1 antibody. (FIGS. 6K-6P) Mitochondrial HK1 associates with DDOST. (FIG. 6K) Representative immunoblots of immunoprecipitated protein samples of HUVEC overexpressing eGFP, FL-I-HK1, and Tr-HIK1 with anti-DDOST antibody. (FIG. 6L) A summary of densitometry in eGFP/DDOST in FIG. 6K. (FIG. 6M) Representative lectin-blot with ConA-HRP of protein samples of HUVEC treated with several concentrations of tunicamycin. (FIG. 6N) mRNA levels of Xbpls in HUVEC treated with tunicamycin (n=2). (FIG. 6O) mRNA levels of Hspa5 in HUVEC treated with tunicamycin (n=2). (FIG. 6P) Representative images of tube formation assay in HUVECs treated with different concentrations of tunicamycin. The scale bar indicates 250 m. The bar graphs indicate the number of junctions and total length of branching (n=4 fields per group). Data were analyzed by one-way ANOVA with Tukey's test for multiple group comparison (FIG. 6L) and unpaired Student t test (FIG. 6P).

FIGS. 7A-7R show that increased protein O-GlcNAcylation in ECs from ΔE1HK1 mice causes defect in angiogenic function. (FIG. 7A) Representative immunoblots of protein samples from isolated ECs from the heart of WI and ΔE1HK1 mice. The bar graph indicates a summary of densitometry (n=3). (FIG. 7B) Representative immunoblots of protein samples from isolated ECs from the heart of C57BL/6J mice treated with HFD_L-NAME. The bar graph indicates a summary of densitometry (n=3). (FIG. 7C) Representative immunoblots of protein samples of isolated ECs from the heart. The bar graph indicates a summary of densitometry (n=3). (FIG. 7D) Representative images of immunofluorescence staining of human hearts with or without DD probed with O-GlcNAcylated proteins (green), WGA (red) and DAPI (blue). The scale bars indicate 25 m. (FIG. 7E) A summary of the intensity of O-GlcNAcylated proteins on capillaries (n=60-90 capillaries). (FIG. 7F) Representative images of tube formation assay in HUVEC treated with FL-HK1 or Tr-HK1. The scale bar indicates 250 km. (FIG. 7G) Bar graphs indicating the number of junctions and total length of branching from panel (F) (n=2-4 fields per group). (FIG. 7H) Representative images of tube formation assay in isolated ECs treated with vehicle or 20 μM of ST045849. The scale bar indicates 250 μm. (FIG. 7I) Bar graphs indicating the number of junctions and total length of branching (n=4 fields per group). (FIG. 7J) Representative images of immunoprecipitated protein samples of isolated ECs with anti-Sp1 antibodies. (FIG. 7K) Representative images of immunoprecipitated protein samples of isolated ECs with anti-OGT antibodies. (FIG. 7L) Representative immunofluorescent images of HUVEC overexpressing FL-HK1 or Tr-HK1 conjugated with eGFP probed with OGT (red) and DAPI (blue). The scale bar indicates 50 m. The bar graph indicates Pearson's R values of localization of eGFP and OGA from the fluorescent images (n=14-18 cells per group). FIGS. 7M-7P. Characterization of HUVEC with stable overexpression of FL-HK1-BioID2-HA and Tr-HK1-BioID2-HA. (FIG. 7M) Schematic of structures of FL-HK1-BioID2-HA and Tr-HK1-BioID2-HA overexpressed in HUVEC. ((FIG. 7N) Representative immunoblot images of protein samples from HUVECs stably overexpressing FL-HK1-BioID2-HA and Tr-HK1-BioID2-HA with anti-eGFP, HK1, and GAPDH antibodies. (FIG. 7O) Representative images of blots immobilized with streptavidin-conjugated HRP and with ponceau S staining. Black and red arrows indicate bands of proteins highly biotinylated by FL-HK1-BioID2-HA or Tr-HK1-BioID2-HA overexpression for each, highlighting the difference in the pattern of protein biotinylation among the groups. (FIG. 7P) Representative immunofluorescent images of cells with HK1-eGFP (green), ATP synthase-0 (red), and DAPI (blue). The scale bar indicates 25 m. The bar graph indicates Pearson's R values in localization of HK1 and ATP synthase-0 in the fluorescent images (n=29-35 cells per group). Data was analyzed by unpaired Student t test. (FIG. 7Q) Representative immunoblots of biotinylated protein samples of HUVEC overexpressing FL-HK1-Bio1D2-HA or Tr-HK1-BioID2-HA pulled down by streptavidin-conjugated beads. (FIG. 7R) The bar graphs indicate summary of the densitometry in FIG. 7Q (n=3-4 per group). Data were analyzed by unpaired Student/test (FIGS. 7A, 7B, 7C, 7E, 7G, 7L, and 7R) or two-way ANOVA (I) with Tukey's test for multiple group comparison.

FIGS. 8A-8V show that EC-specific overexpression of OGA reverses the HFpEF phenotype. (FIG. 8A) Schematic of the strategy to overexpress OGA in Tie2-Tet-OGA mice. (FIG. 8B) Representative images of immunoblot with protein samples isolated from Tie2-Tet-OGA mice with or without DOX treatment. (FIG. 8C) Summary of OGA expression in FIG. FIG. 8B) (n=4). (FIG. 8D) Representative immunoblots of protein samples of isolated ECs from the heart of these mice. (FIG. 8E) Bar graph summary of densitometry (n=3 per group) in panel (FIG. 8D). (FIG. 8F) Bar graph summary of EF, LVDd and PWTd (n=7-8 mice per group). (FIG. 8G) HW divided by length of tibial bones (TL). (FIG. 8H) Representative images of TMF and bar graph of E/A (n=7-8 mice per group) The vertical scale bar indicates 400 mm/s, and the horizontal one indicates 0.1 sec. (FIG. 8I) Representative images of tissue doppler imaging (TDI) (n=7-8 mice per group). The vertical scale bar indicates 30 mm/s, and the horizontal one indicates 0.1 sec. (FIG. 8J) Bar graph summary of the LA area (n=7 nice per group). (FIG. 8K) Representative immunofluorescent images of hearts probed with WGA (red) and DAPI (blue). The scale bar indicates 25 m. The brightness of WGA is modified in magnified images so that the number of capillaries can be compared among the groups. (FIG. 8L) Summary of CM cross sectional area (CSA) (60 CMs per mouse. n=3 mice per group). (FIG. 8M) Summary of capillary density (3 images per mouse. n=3 mice per group). (FIG. 8N) Summary of WGA fluorescence intensity on capillaries from heart (20 capillaries per image. 3 images per mouse. n=3 mice per group). FIGS. 8O-8T show echocardiography in ΔE1HK1 crossed with Tie2-Tet-OGA mice. (FIG. 8O) EF, (FIG. 8P) % FS, (FIG. 8Q) IVSd, (FIG. 8R) PWTd, (FIG. 8S) LVDd, and (FIG. 8T) LVDs in mice of WT, AE1HK1 or AE1HK1 plus DOX treatment (all groups hold Tie2-Tet-OGA transgene); (n 4). Data were analyzed by one-way ANOVA with Tukey's test for multiple group comparison. (FIG. 8U) Representative images of TDI of AE1HK1 crossed with Tie2-Tet-OGA mice. The vertical scale bar indicates 30 mm/s, and the horizontal one indicates 0.1 sec. (FIG. 8V) Summary of e' and E/e' (n=6-9 per group) of FIG. 8L. Data were analyzed by unpaired Student t test (C) or one-way ANOVA with Tukey's test for multiple group comparison (FIGS. 8E, 8F, 8G, 8H, 8J, 8L, 8M, 8N and 8V).

FIGS. 9A-9X illustrate that pharmacological inhibition of OGT reverses the HFpEF phenotype. (FIG. 9A) Representative immunoblot and membrane stained with Ponceau S of protein samples of tECs isolated after treatment of 0, 10, 20, 30 or 40 rg/kg of 5S-GlcNHex. (FIG. 9B) BW of C57BL6/J mice treated with or without HFD_L-NAME, and 20 mg/kg or 30 mg/kg of 5S-GlcNHex. (n=4-7 mice per group). (FIG. 9C) Bar graph summary of sBP (n=3-7 mice per group). (FIG. 9D) Schematic of the strategy to treat C57BL/6J mice with NC and HFD_LNAME with and without 5S-GlcNHex. (FIG. 9E) Representative images of M-mode of LVs. The vertical scale bar indicates 2 mm, and the horizontal one indicates 0.1 sec. (FIG. 9F) Bar graph summary of EF and LVDd. (n 5-7 mice per group). (FIG. 9G) Representative Doppler images of TMF. The vertical scale bar indicates 400 mm/s, and the horizontal one indicates 0.1 sec. (FIG. 9H) Summary bar graph of E/A ratio (n=5-7 mice per group). (FIG. 9I) Summary bar graph of LA size (n=5-7 mice per group). (FIG. 9J) HW divided by TL (n=5-8). (FIG. 9K) Summary bar graph of running distance in treadmill exercise tests. (n=5-7 mice per group). (FIG. 9L) Schematic of how HK1 dislocation from mitochondria affects protein O-GlcNAcylation and induce EC dysfunction and the development of HFpEF. Data were analyzed by one-way ANOVA with Tukey's test for multiple group comparison. FIGS. 9M-9X show 50 mg/kg of 5S-GlcNHex reverses the HFpEF phenotype. (FIG. 9M) Representative immunoblots of protein samples ofECs isolated after treatment of 50 mg/kg 5S-GleNHex. (FIG. 9N) Time course of 1BW in C57BL/6J mice treated with 50 mg/kg 5S-GIcNIex every 2 days. (FIG. 9O) Representative images of M-mode ofLVs. The vertical scale bar indicates 1 mm, and the horizontal one indicates 0.1 sec. (FIG. 9P) Bar graph summary of EF. (n=5-9 mice per group). (FIG. 9Q) Bar graph summary of LVDd. (n=5-9 mice per group). (FIG. 9R) Bar graph summary oftPWTd. (n=5-9 mice per group). (FIG. 9S) HW divided by TL (n=8-10). (FIG. 9T) Representative images of TMF. The vertical scale bar indicates 400 mm/s, and the horizontal one indicates 0.1 sec. (FIG. 9U) Summary bar graph of E/A (n=5-9 mice per group). (FIG. 9V) Representative images TDI. The vertical scale bar indicates 30 mm/s, and the horizontal one indicates 0.1 sec. (FIG. 9W) Summary bar graph of e', E/e'. (n=5-9 mice per group). (FIG. 9X) Summary bar graph of LA area (n=5-9 mice per group). Data were analyzed by one-way ANOVA with Tukey's test for multiple group comparison.

### DETAILED DESCRIPTION

Disclosed are methods and compositions for treating or preventing heart failure with preserved ejection fraction (HFpEF). The disclosed methods include treatment of heart failure, namely heart failure with preserved ejection fraction (HFpEF), and protection against such heart failure. The methods include treatment and protection against mechanisms associated with heart failure, such as defects in endothelial cells in the heart. Defects in endothelial cells that may be treated or protected may include defective angiogenic function and/or a shift in glycolysis activity in the endothelial cells of the heart. An aspect of the disclosure is methods of administering to a subject who at risk of experiencing or has experienced HFpEF a composition that treats or prevents a defect in endothelial cells in the heart of the subject.

The methods and compositions may be described using the following definitions.

Unless otherwise specified or indicated by context, the terms “a” “an”, and “the” mean one or more.” In addition, singular nouns should be interpreted to mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤;10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The terms “subject” or “patient” may be used interchangeably throughout the disclosure and may refer to human or non-human animals.

One aspect is a method of treating or preventing heart failure with preserved ejection fraction (HFpEF) comprising administering a composition comprising an O-linked N-acetylglucosamine transferase (OGT) inhibitor. The OGT inhibitor is effective to reduce or eliminate excessive protein O-GlcNAcylation in ECs, which is associated with HFpEF. Preferably, the OGT inhibitor is effective for in vivo applications when administered to a subject with or at risk of developing HFpEF. In embodiments, the OGT inhibitor may be 2-deoxy-2-N-hexanamide-5-thio-d-glucopyranosiade (5S-GlcNHex), which reduces protein O-GlcNAcylation in ECs in vivo. Reducing protein O-GlcNAcylation in ECs in the heart of a subject with HFpEF decreases or eliminates cardiac hypertrophy and microvascular rarefaction, and restores or maintains diastolic function in the subject. An embodiment of the disclosure is a method of administering an OGT inhibitor to a subject experiencing or at risk of experiencing HFpEF, thereby reducing protein O-GlcNAcylation in ECs in the heart of the subject.

ECs rely on glycolysis to produce energy and perform their physiological functions (Li et al., 2019). ECs generate >85% of their ATP through glycolysis even in oxygen-replete conditions (De Bock et al., 2013). Hexokinases (HKs) carry out the first step in glycolysis by phosphorylating glucose to glucose-6-phosphate, thereby committing it to various downstream metabolic pathways. HK1, and HK isozyme, exhibits predominant expression within ECs relative to other cardiac cell types, and dislocates from mitochondria in HFpEF. The studies in the examples demonstrate that HK1 dislocation from mitochondria has a role in increasing protein 0-GlcNAcylation in ECs. The increased protein O-GlcNAcylation caused by mitochondrial HK1 dislocation results in EC dysfunction. In embodiments, HK1 dislocation from mitochondria mediates protein O-GlcNAcylation in ECs in a subject with HFpEF.

Another aspect is a method of treating or preventing HFpEF by administering a composition that increases O-linked N-acetylglucosaminase (OGA) in endothelial cells of the heart. Increased OGA in endothelial cells is associated with reducing protein O-GlcNAcylation in ECs. In embodiments, reducing protein O-GlcNAcylation in ECs with overexpression of OGA decreases or eliminates cardiac hypertrophy and microvascular rarefaction. In embodiments, reducing protein O-GlcNAcylation in ECs restores or maintains diastolic function in a heart with HFpEF. In embodiments, reducing protein O-GlcNAcylation in ECs with overexpression of OGA maintains the angiogenic function of ECs, which results in preventing the development of HFpEF. In embodiments, reducing protein O-GlcNAcylation in ECs with overexpression of OGA restores angiogenic function of ECs, for example in ECs of the heart.

The disclosed compositions may be used in methods to treat or prevent heart failure in a subject, particularly HFpEF. The compositions may comprise an enzyme inhibitor, such as an O-linked N-acetylglucosamine transferase (OGT) inhibitor that may treat HFpEF. The compositions may comprise an enzyme, such as O-linked N-acetylglucosaminase (OGA), that may treat HFpEF or prevent HFpEF. The compositions may comprise an enzyme, such as OGA, that alters or stimulates mechanisms that lead to HFpEF. For example, OGA may prevent angiogenic dysfunction in endothelial cells in the heart that is associated with developing HFpEF.

The disclosed compositions comprising an enzyme or enzyme inhibitor, for example OGA enzyme or OGT inhibitor, may be formulated as a pharmaceutical composition for administration to a subject. Such compositions may be administered to a subject in need thereof to treat or prevent HFpEF A subject “in need of” treatment or “in need thereof” referring to a treatment according to the present disclosure may be suffering from, suspected of suffering from, or at risk of suffering from a specific disease or disorder, such as HFpEF, may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily distinct groups.

Such pharmaceutical compositions can be formulated and/or administered in dosages and by techniques well known to those skilled in the medical arts taking into consideration such factors as the age, sex, weight, and condition of the particular patient, and the route of administration. In some embodiments, a pharmaceutical composition can be formulated and/or administered in an effective amount or therapeutically effective amount to treat or prevent a disease, disorder, or condition. The term “effective amount” as used herein generally refers to a concentration or amount of a compound, material, or composition, as described herein, that is effective to achieve a particular biological result.

The compositions may include pharmaceutical solutions comprising carriers, diluents, excipients, and surfactants as known in the art. Further, the compositions may include preservatives. The compositions also may include buffering agents.

The pharmaceutical compositions may be administered therapeutically. In therapeutic applications, the pharmaceutical compositions are administered to a patient in an amount sufficient to elicit a therapeutic effect, e.g., a therapeutic effect in response to a heart failure or a mechanism that may lead to heart failure such as tissue or cell defect in a heart (i.e., a “therapeutically effective dose”).

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” may refer to the amount or dose of the compound or composition, upon single or multiple dose administration to the subject, which provides the desired effect in the subject under diagnosis or treatment. The disclosed methods may include administering an effective amount of the disclosed compositions (e.g., as present in a pharmaceutical composition) for treating or preventing HFpEF.

### Examples

Heart failure (HF) with preserved ejection fraction (HFpEF) is associated with heightened all-cause mortality (Bhatia et al., 2006; Fonarow et al., 2007; Kardys et al., 2009), however, the precise molecular mechanisms driving the HFpEF pathogenesis remain poorly understood. HFpEF is more prevalent with older age (Xu and Daimon, 2016), and frequently coexists with multiple comorbidities such as hypertension, renal dysfunction, and diabetes (Dhingra et al., 2014; Haass et al., 2011). There are few treatment options for HFpEF, and current therapies primarily address symptom relief and target comorbid conditions. Evidence suggests that endothelial cell (EC) dysfunction plays a pivotal role in the development of HFpEF (Mohammed et al., 2015; Paulus and Tschope, 2013). A recent prospective multi-center study reported that microvascular dysfunction (MVD) is prevalent in 69-81% of HFpEF patients (Shah et al., 2018), and MVD is linked to worse diastolic dysfunction and 2.47 times increase in the major adverse cardiovascular events (Taqueti et al., 2018). Moreover, EC dysfunction exhibits a positive correlation with exercise intolerance in HFpEF patients (Borlaug et al., 2010; Farrero et al., 2014). However, the mechanism underlying the observed EC dysfunction and the causal relationship between EC dysfunction and HFpEF development remain elusive. Notably, two major risk factors for the development of HFpEF, diabetes and hypertension, are both associated with increased protein O-GlcNAcylation in ECs (Lima et al., 2010; Makino et al., 2015). Consequently, it is conceivable that HFpEF pathophysiology may involve increased protein O-GlcNAcylation in ECs as a consequence of predisposing risk factors.

ECs highly rely on glycolysis to produce energy and perform their physiological functions (Li et al., 2019), as they generate>85% of their ATP through glycolysis even in oxygen-replete conditions (De Bock et al., 2013). Hexokinases (HKs) carry out the first step in glycolysis by phosphorylating glucose to glucose-6-phosphate, thereby committing it to various downstream metabolic pathways (Abel et al., 1999; Bell et al., 1993; Gould and Holman, 1993; Mueckler, 1994; Printz et al., 1997). While glycolysis yields energy-producing intermediates, it can also be shunted into other pathways, including the pentose phosphate pathway (PPP), hexosamine biosynthesis pathway (HBP), or be converted to glucose-1-phosphate for glycogen synthesis (Adeva-Andany et al., 2016). In mammals, five distinct HK isozymes (HK1, HK2, HK3, HK4/glucokinase, and HK5/HKDC1) have been identified, each exhibiting unique tissue expression, subcellular localization, kinetics, and substrate specificities (Middleton, 1990; Ureta, 1982; Wilson, 2003). Notably, HK1 and HK2 contain a mitochondrial binding domain (MBD) at their N-terminus, which enables outer mitochondrial membrane (OMM) binding (Sui and Wilson, 1997). Prior studies have proposed several HK1/HK2 mitochondrial binding functions, including regulation of opening of the permeability transition pore (Sun et al., 2008) and recruitment of apoptotic mediators (Pastorino et al., 2002). More recently, the inventors' research unveiled a novel role of HK1 dislocation from mitochondria in macrophages, redirecting glycolytic metabolites into PPP and promoting the production of proinflammatory cytokines (De Jesus et al., 2022).

This disclosure shows that HK1 exhibits predominant expression within ECs relative to other cardiac cell types and dislocates from mitochondria in the setting of HFpEF. Furthermore, the disclosed studies demonstrate that HK1 dislocation from mitochondria is sufficient to cause HFpEF in murine models, with EC dysfunction preceding other pathological changes in the hearts. The studies further unveil HK1 subcellular localization as a critical determinant that modifies the allocation of HBP intermediates between N-glycosylation and O-GlcNAcylation machineries. Importantly, increased protein O-GlcNAcylation caused by mitochondrial HK1 dislocation results in EC dysfunction. Finally, EC specific overexpression of O-GlcNAcase (OGA) and pharmacological inhibition of O-linked N-acetylglucosamine transferase (OGT) effectively mitigates cardiac phenotypes in murine HFpEF models is shown. Collectively, the disclosed findings underscore the significant role of HK1 mitochondrial binding in orchestrating HBP metabolites to be shuttled among protein glycosylation machineries, providing a promising therapeutic pathway for HFpEF by targeting the elevation of protein O-GlcNAcylation.

While numerous clinical studies suggested the involvement of EC dysfunction in the development of HFpEF (Shah et al., 2018; Taqueti et al., 2018), the exact causal relationship and the underlying molecular mechanisms are not well understood. In these disclosed studies, a causal relationship of EC dysfunction and the development of HFpEF is demonstrated, with experimental findings providing evidence supporting the pivotal role of EC dysfunction in HFpEF pathogenesis. The studies effectively mitigate EC dysfunction and the HFpEF phenotype in mice subjected to HFD_L-NAME treatment or in the AE1HK1 mice by suppressing protein O-GlcNAcylation in ECs. The results imply that the upsurge in protein O-GlcNAcylation and the consequential EC dysfunction constitute critical driving force in the pathogenesis of HFpEF. Additionally, increased dislocation of HK1 from mitochondria in ECs under HFpEF conditions is shown, despite the lack of any notable changes in its expression. This phenomenon is particularly intriguing given the previously established roles of HK1 mitochondria binding in other diseases, including its contribution to the development of Alzheimer's disease via the reduction of ATP production (Rossi et al., 2020), as well as its association with low-grade inflammation seen in conditions like aging or diabetes mellitus within macrophages (De Jesus et al., 2022). These findings unveil a critical molecular mechanism underlying the pathogenesis of HFpEF, wherein HK1 dislocates from mitochondria in ECs in the setting of HFpEF and it is sufficient to induce HFpEF through increased protein O-GlcNAcylation and EC dysfunction.

Protein O-GlcNAcylation regulates multiple cellular processes in metazoans (Yang and Qian, 2017). The orchestration of O-GlcNAcylation is primarily governed by OGT and OGA, both intricately regulated by various mechanisms, including protein abundance and glucose availability. Nevertheless, significant disparities in their protein levels between WT and AE1HK1 ECs was not observed. Another critical determinant of O-GlcNAcylation resides in the availability of UDP-GlcNAc, a crucial substrate that hinges upon the cellular nutrient status and it is considered as a sensor of nutrient deficiency (Akella et al., 2019; Chatham et al., 2021). Metabolomic and gene expression analyses have shown that the augmentation of O-GlcNAcylation is not induced by conventional pathway modifications. Instead, it emerges as a consequence of the subcellular repositioning of HK1, catalyzing a redirection of HBP-intermediates away from the N-Glycosylation machinery towards O-GlcNAcylation. This paradigm shift in substrate allocation underscores the significance of metabolic enzyme subcellular localization alterations, as exemplified by HK2's association with cytosolic actin filaments, supplying glycolytic ATP for cellular motility and angiogenesis (De Bock et al., 2013), and the transient migration of TCA cycle enzymes into the nucleus, thereby ensuring the efficient supply of acetyl-CoA and α-KG to epigenetic machinery (Nagaraj et al., 2017).

The N-glycosylation machinery is ensconced within the ER and the Golgi apparatus, which is spatially separated from where the O-GlcNAcylation machinery reside. An association between mitochondrial HK1 and DDOST, a subunit of oligosaccharide transferase which transfers glycans to newly synthesized proteins in the ER is demonstrated. ER and mitochondria interact and form a structure called mitochondria-associated membranes (MAM) (Rieusset, 2018) through a number of proteins, including voltage-dependent anion channel (VDAC) (Rizzuto et al., 2012). Through its interaction with VDAC, HK1 may establish a close spatial relationship with the ER, thus enabling the efficient provision of sugar substrates to the N-glycosylation machinery. Consistent with this model, itraconazole, an antifungal agent known for its ability to dislocates HK1 from the mitochondria, has also been demonstrated to impede the N-glycosylation of VEGFR2 in ECs (Nacev et al., 2011).

The disclosed studies demonstrate that dislocated HK1 exhibits a heightened propensity to colocalize with OGT within the nucleus and cytosol in contrast to mitochondrial HK1 in ECs. The molecular-level proximity between Tr-HK1 and key players of O-GlcNAcylation such as glutamine—fructose-6-phosphate transaminase 1 (GFPT1) supported by BioID2 assays suggest that dislocated HK1 actively localizes itself alongside the O-GlcNAcylation machinery, thereby bolstering the supply of critical substrates for this intricate biochemical reaction. The fact that overexpression of Tr-HK1 augmented O-GlcNAcylation levels, coupled with the preservation of MMP in ΔE1HK1 ECs, collectively support that this enhancement in protein O-GlcNAcylation is not attributed to disturbed mitochondrial function, but rather reflects the gain of function of dislocated HK1. Overall, these results underscore the pivotal role of HK1 subcellular localization as a key player for governing the supply of HBP substrates to downstream glycosylation machinery, thus balancing protein N-glycosylation and O-GlcNAcylation within ECs.

Developing animal models that recapitulate the clinical features of HFpEF is of great interest in cardiovascular research. Current HFpEF models employ a multiple perturbations like aging, HFD and compounds like L-NAME, AngII or desoxycorticosterone pivalate (Deng et al., 2021; Withaar et al., 2021a; Withaar et al., 2021b). While these multiple hit approaches allow these models to encompass HFpEF's associated comorbidities, it simultaneously presents challenges in characterizing the underlying molecular mechanisms because of the systemic and synergistic effects of these interventions. In contrast, the AE1HK1 mice provides several advantages for studying HFpEF. Notably, these mice spontaneously develop HFpEF as they age, affording a unique opportunity to study the disease progression without external inductions. Additionally, the AE1HK1 mice exhibit an accelerated development of HFpEF when subjected to HFD, further enhancing the model's versatility. Importantly, this model exerts diastolic dysfunction in female mice, in addition to male animals. These attributes collectively render the AE1HK1 mouse model an invaluable tool for unraveling the intricate molecular underpinnings of HFpEF.

It is noteworthy that mice with EC-specific overexpression of OGA reverse the HFpEF phenotype, thus indicating the critical role of increased O-GlcNAcylation in ECs. Additionally, the inventors previously reported that macrophages from ΔE1HK1 mice produce augmented cytokines in response to lipopolysaccharide (LPS) administration (De Jesus et al., 2022). However, the heightened cytokine production in macrophages is solely provoked by LPS treatment and does not occur under baseline condition. Thus, it is unlikely that functional changes induced by HK1 dislocation from mitochondria in macrophages significantly contribute to the development of HFpEF in the ΔE1HK1 mice.

Numerous potential targets for O-GlcNAcylation in the context of the EC function exist. For instance, increased O-GlcNAcylation of AKT in ECs has been shown to inhibit its signaling cascade, resulting in reduced angiogenesis (Federici et al., 2002; Luo et al., 2008). Similarly, endothelial nitrogen oxide synthase eNOS is subjected to O-GlcNAcylation, and its activity is downregulated in response to hyperglycemia (Du et al., 2001), suggesting that excessive O-GlcNAcylation of certain proteins in ECs leads to detrimental effects on angiogenesis. Furthermore, since dislocated HK1, in addition to its cytoplasmic presence, localizes within the nucleus, it is conceivable that this subcellular localization of HK1 may lead to the O-GlcNAcylation of specific nuclear proteins, orchestrating the machinery of angiogenesis in ECs. This hypothesis finds support in prior studies that have reported interactions between OGT and multiple epigenetic factors, thereby implicating O-GlcNAcylation in the regulation of epigenetic processes and gene expression (Dehennaut et al., 2014; Hanover et al., 2012; Lewis and Hanover, 2014; Singh et al., 2015).

The majority of currently available OGT inhibitors face limitations for in vivo applications due to challenges related to cellular permeability (ability to get into the cell) and solubility (Ju Kim, 2020). However, 5S-GlcNHex, an OGT inhibitor that successfully reduces protein O-GlcNAcylation was introduce in ECs in vivo, leading to the reversal of the HFpEF phenotype. Therefore, the study results indicate that this compound may offer a novel therapeutic approach that targets a previously unexplored molecular pathway in HFpEF, suggesting that this chemical may provide a potential treatment strategy for this disease.

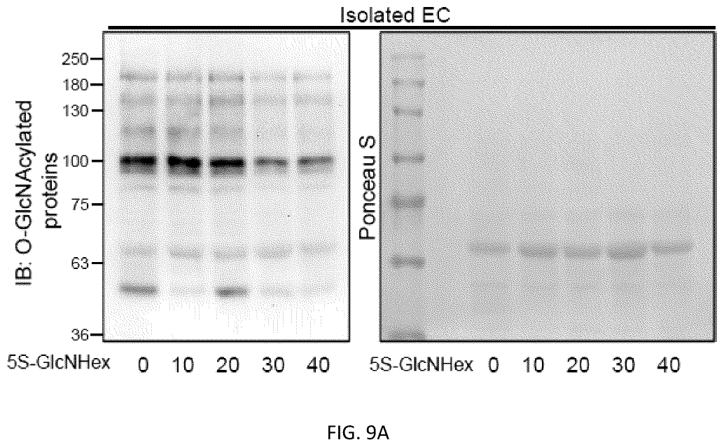

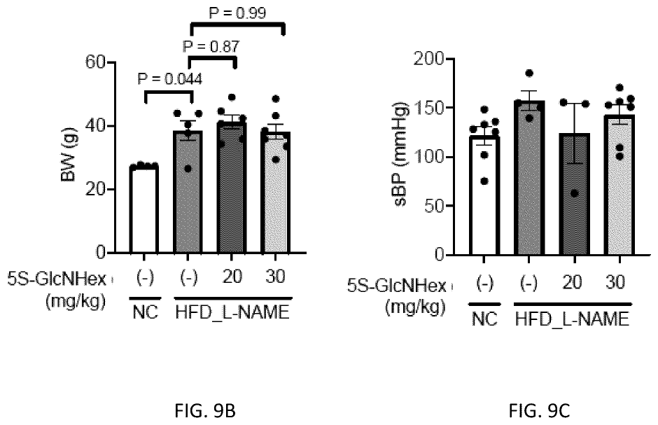

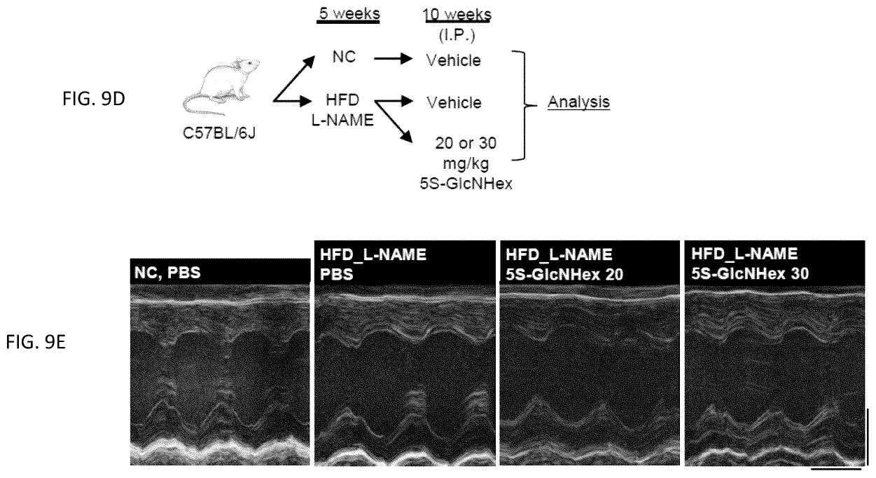

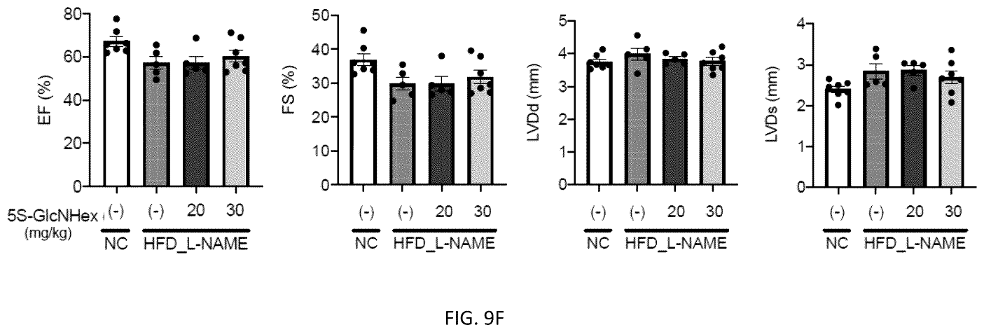

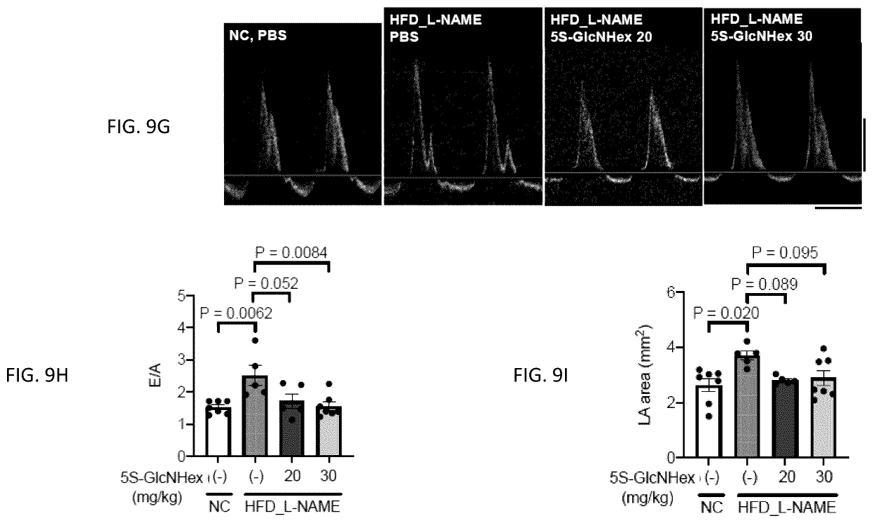

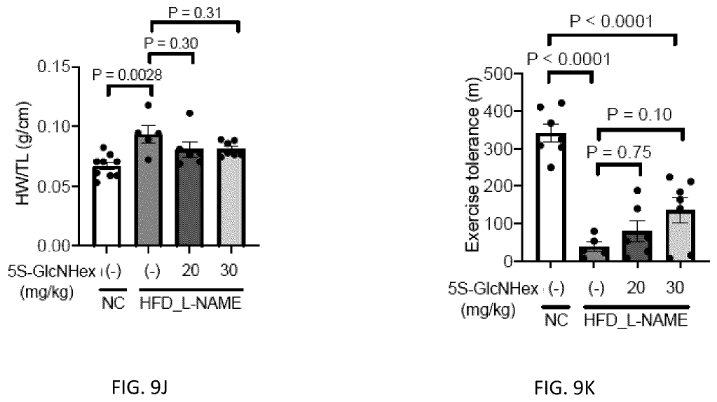

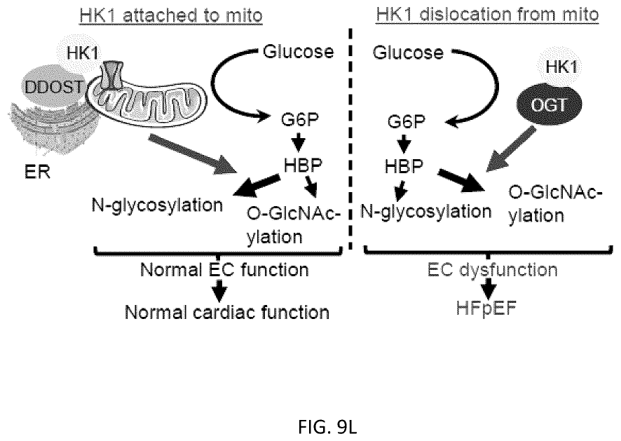

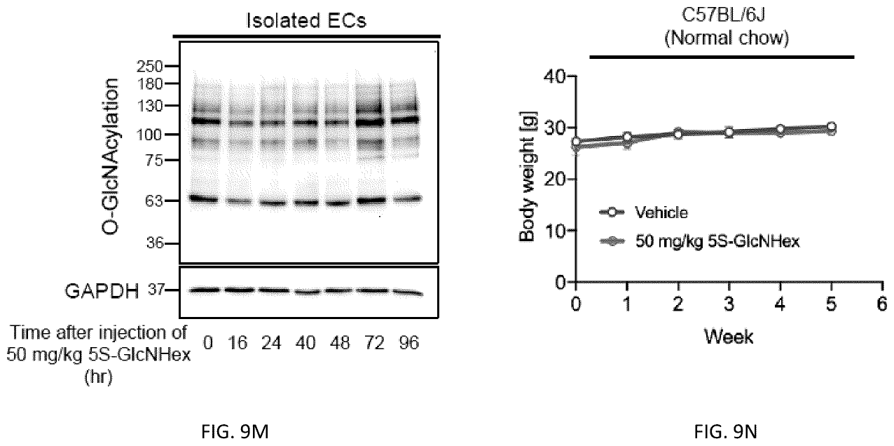

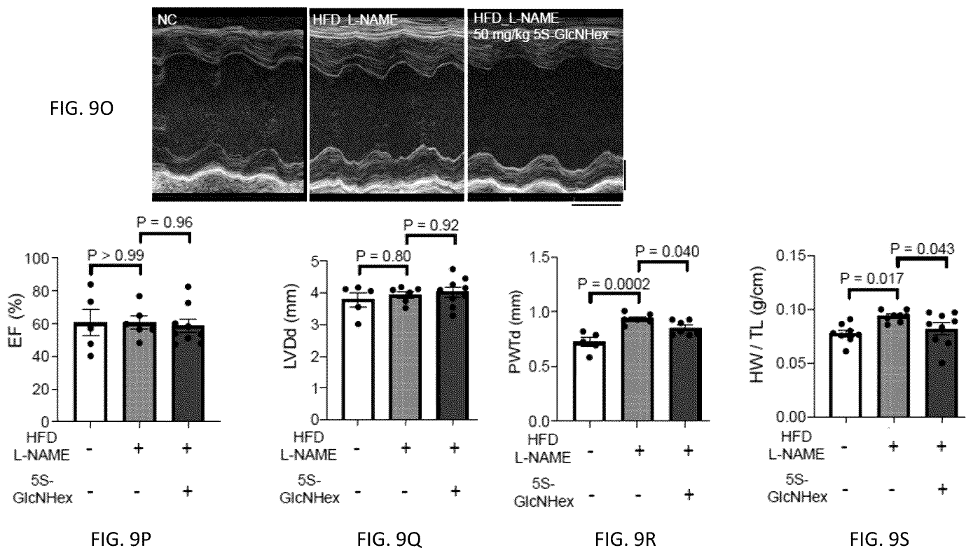

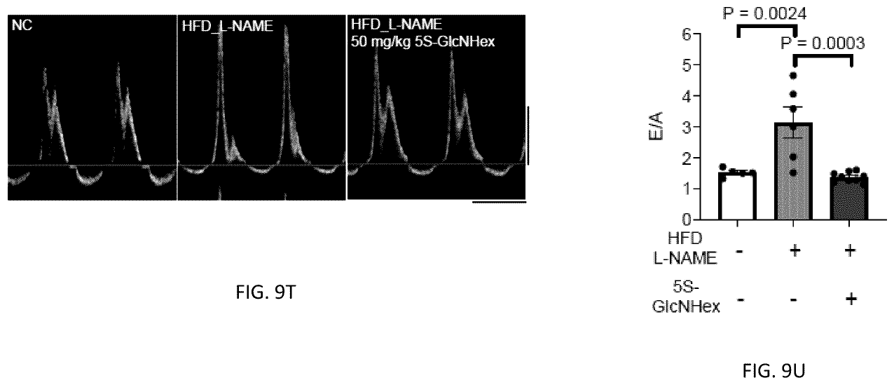

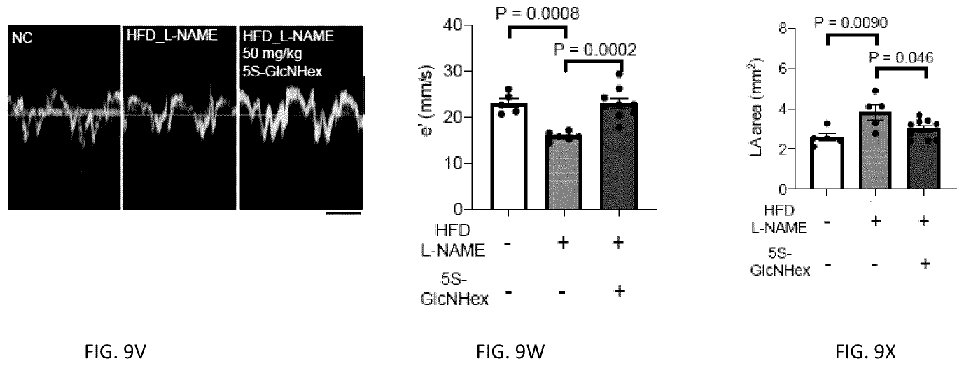

These studies reveal a crucial role for HK1 mitochondrial localization in the fate of HBP intermediates, directing them towards either N-glycosylation or O-GlcNAcylation machinery within ECs. The resultant hyper-O-GlcNAcylation leads to impairments in the angiogenic potential of ECs, a pivotal factor in the pathogenesis of HFpEF (FIG. 9L). These findings not only establish a causal link between endothelial hyper-O-GlcNAcylation and HFpEF development but also underscore the pivotal significance of this molecular pathway in the disease's pathogenesis. The disclosed studies open up an encouraging avenue for potential therapeutic interventions in HFpEF, with the potential to significantly enhance its management and improve patient outcomes.

### Materials and Methods

### Mouse Studies

HK_{ΔE1HK1/Δ}^{E1HK1} (ΔE1HK1) and HK1^{WT/WT} wild-type (WT) control mice were generated by the Northwestern Mutagenesis and Transgenic core (De Jesus et al., 2022). Briefly, guide RNAs (gRNAs) were designed to induce a double-strand break (DSB) near the 5′ ATG start codon of HK1 in exon 1, and a DNA template containing an M-DYKDDDDK (Methionine-FLAG-tag) sequence with flanking homology arms to the HK1 gene near exon 1 was introduced into fertilized zygotes and implanted into surrogate mice. Homology-directed repair (HDR) of the DSB resulted in the replacement of the coding region of the HK1 MBD with the methionine-FLAG-tag sequence. Mouse's genetic background is C57BL/6J. Tie2-Tet-OGA mice are generous gifts from Dr. Dillmann and Dr. Makino (Makino etal., 2015). Mice were maintained in the barrier facility at Northwestern University under specific pathogen-free conditions under the Federal and University guidelines and protocols approved by Institutional Animal Care and Use Committee (IACUC) with 12 hr light and 12 hr dark cycles and received normal chow (NC). Male and female ΔE1HK1 mice were used at 8-40 weeks of age. 8-week-old ΔE1HK1 male mice were fed HFD consisting of 60% fat, 20% protein, and 20% carbohydrates for 20 weeks. To produce the HFpEF mice model, C57BL/6J and Tie2-Tet-OGA mice were fed HFD and 0.5 g/L ofN.-Nitro-L-arginine methyl ester (LNAME, Sigma-Aldrich) for 8-15 weeks. For the induction of OGA overexpression, 0.5 g/L of DOX in drinking water was administered to Tie2-Tet-OGA mice until they were sacrificed. All animal studies were approved by the IACUC at Northwestern University and were performed in accordance with guidelines from the National Institutes of Health.

Cardiomyocyte and Fibroblast Isolation from Mouse Hearts

Cardiomyocytes (CMs) and fibroblasts (FBs) were isolated from mouse hearts as previously described (Ackers-Johnson et al., 2016). Briefly, mice were anesthetized and cut open below the diaphragm to expose the heart. After injection of 7 mL EDTA buffer to the right ventricle, the aorta was clamped by forceps and the heart was removed. Multiple rounds of injections to the apex of the heart were performed with the following: 10 mL EDTA buffer, 3 mL of perfusion buffer, and 30 mL of collagenase buffer. The hearts were then pulled apart using forceps and filtered through 100 m strainers to remove large tissue debris. After 3 rounds of gravity settling, the adult CMs were resuspended in culture media. The supernatant obtained from the gravity setting was centrifuged and the pellet was resuspended in culture media as cardiac fibroblasts.

Endothelial Cell Isolation from Mouse Hearts

Endothelial cells (ECs) were isolated from mouse hearts as previously described (Luo et al., 2016; van Beijnum et al., 2008). Briefly, dissected mouse heart was minced and incubated with PBS containing 2% fetal bovine serum (FBS), 1 mg/ml collagenase II (Worthington), and 0.6 U/ml dispase II (Sigma-Aldrich) for 30 mins at 37° C. 7.5 μg/ml of DNase I (Sigma-Aldrich) was added to the digested hearts and additional incubation for 30 minutes was performed. The digested material was filtered through 40 μm cell strainer, then incubated with biotinylated anti-CD45 antibody (Invitrogen) and depleted of CD45 positive cells using EasySep Mouse Streptavidin (STEMCELL technologies) as described in the manufacture's protocol. Then cells were incubated with rat anti-mouse CD31 monoclonal antibody (1 μg/ml, BD) conjugated with magnetic Dynabeads Sheep Anti-Rat IgG (Invitrogen) at room temperature for 20 minutes and then ECs were captured and isolated by a magnet, then cultured in EGM2 (Lonza) media.

### Cell Culture and Reagents

HUVECs were purchased from Lonza and grown in EGM2 media (Lonza). HEK293T cells were purchased from ATCC and cultured in DMEM (Corning) with 10% FBS (Bio-Techne Sales Corp.). All cells were maintained in a 37° C. incubator with 5% CO2 and were 70% to 90% confluent when collected for analyses.

### Immunoblot Analysis

Protein extracts from frozen mouse hearts, isolated cardiomyocytes, fibroblasts, endothelial cells, and HUVECs were prepared by lysis in ice-cold RIPA buffer (150 mM NaCl, 25 mM Tris-HCl pH 7.4, 1% NP-40, 1% sodium deoxycholate, 0.1% SDS) containing protease inhibitors. For analyses of O-GlcNAcylation, 200 μg/ml of PUGNAC (Sigma-Aldrich) was added to the RIPA buffer. Proteins were separated by sodium dodecyl sulfate-polyacrylamide gel electrophoresis (SDS-PAGE) on 8 or 10% polyacrylamide gels and transferred to nitrocellulose membranes. Membranes were incubated with the following primary antibodies overnight, before the addition of HRP-conjugated secondary antibodies: HK1 (2024, Cell Signaling Technology); HK2 (2867, Cell Signaling Technology); GAPDH (60004-1-Ig, Proteintech); FLAG (F1804, Sigma-Aldrich); alpha-tubulin (66031-1-Ig, Proteintech); ATP synthase C (ab181243, Abcam); Phospho-AMPKα (Thr172) (4188, Cell Signaling Technology); AMPKα (2603, Cell Signaling Technology); GFPT1 (14132-1-Ig, Proteintech); RPN1 (12894-1-AP, Proteintech); RPN2 (10576-1-AP, Proteintech); DDOST (14916-1-AP, Proteintech); GFP (66002-1-Ig, Proteintech), O-Linked N-Acetylglucosamine (ab2739, Abcam); OGT (11576-2-AP, Proteintech); OGA (66033-1-Ig, Proteintech). The presence of target protein was visualized using Super Surgical Western Pico ECL substrate (Pierce). Quantification of the western blotting images was done using ImageJ (NIH).

### Reverse Transcription and Quantitative Realtime PCR

RNA was isolated from cells or heart samples using RNA-STAT60 (Tel-Test), and reverse transcribed with Taqman Reverse Transcription Reagents (Invitrogen) according to manufacturers' instructions. Quantification of relative gene expression was done using Fast SYBR Green Master Mix (Applied Biosystems) and run on 7500 Fast Real-Time PCR system (Applied Biosystems). The relative gene expression was determined using differences in Ct values between the gene of interest and house-keeping control genes, which are Actb, Hprt1, and/or 18S, depending on the experiment.

### Preparation of Organ Sections for Histology and Immunofluorescence

Mice were anesthetized with a 250 mg/kg dose of freshly prepared Tribromoethanol. After making a small incision in the inferior vena cava, the beating heart was perfused with relaxing buffer (100 mM KCl, 5 mM EGTA, and 5 mM Na pyrophosphate) until termination of beating, followed immediately with 30 ml of 4% paraformaldehyde (PFA) using the peristaltic pump (7 ml/min) from the apex of the left ventricle (LV) through 22G needle. The excised heart tissue was further fixed overnight in 4% PFA. The heart was cut in half, and one of them was dehydrated in 70%, 80%, 90%, and 100% ethanol. The tissue sample was further dehydrated with xylene and embedded into paraffin. Sections were stained with hematoxylin and eosin for evaluation of general cardiac morphology and tissue organization. Masson's Trichrome (MT) staining was used to visualize cardiac fibrosis. Another half of the fixed heart was incubated with 15% and 30% sucrose in PBS for cryoprotection, then was embedded into OCT compound, followed by freezing down in 2-methyl butane cooled in dry ice and liquid nitrogen. Frozen sections were subsequently used for immunofluorescent experiments.

### Immunofluorescence

Cells grown on glass-bottom plates were fixed with 4% PFA for 15 min. The cells or frozen sections of organs were then washed with PBS, blocked with 5% goat serum and 0.1% Triton X in PBS for 60 min, and incubated overnight with antibodies for HK1 (2024, Cell Signaling Technology), CD31 (553370, BD), ATP synthase beta conjugated with Alexa 555 (MA1930A555, Thermo Fisher Scientific) and OGT (11576-2-AP, Proteintech) in 1% BSA, 0.1% Triton X in PBS or 5 μg/ml wheat germ agglutinin (WGA) conjugated with Alexa™Fluor 594 (Invitrogen) overnight at 4° C. The bound antibodies were labeled with an Alexa Fluor(R) 488 or 594 anti-rabbit secondary antibodies (111-545-144 and 111-585-144, Jackson ImmunoResearch) or an Alexa Fluor(R) 647 anti-rat secondary antibody (111-605-003, Jackson ImmunoResearch). Nuclei were stained with DAPI in ProLong® Gold Antifade Mountant (P36931, Invitrogen). Fluorescent images were visualized using a Nikon W1 Dual Cam Spinning Disk Confocal with TIRE Colocalization analysis was performed using a plug-in of Coloc2 in ImageJ, and Pearson's R-value was calculated. A histogram of gray value on a line of region of interest (ROI) was also obtained using ImageJ.

### Protein Fractionation

Protein samples of cytosolic and membrane fractions were obtained as described previously (Baghirova et al., 2015). In brief, 40 mg of fresh heart tissue was homogenized in 250 L of PBS by a tissue homogenizer, followed by centrifugation in QIAshredder homogenize (Qiagen) at 500 xg for 10 min at 4° C. The pellet in the filtrate was resuspended in 250 μL of Lysis buffer A (150 mM NaCl, 50 mM HEPES pH 7.4, 25 μg/mL digitonin [Sigma-Aldrich], 1M hexylene glycol, and 1% protease arrest), and incubated on an end-over-end rotator for 10 min at 4° C. The samples were centrifuged at 4000×g for 10 min at 4° C., and the supernatant was stored as samples containing cytosolic proteins. The pellet was resuspended in 200 μL of Lysis buffer B (150 mM NaCl, 50 mM HEPES pH 7.4, 1% igepal® [Sigma-Aldrich], 1M hexylene glycol, and 1% protease arrest) and incubated on an end-over-end rotator for 3 hr at 4° C. The suspension was centrifuged at 6800×g for 10 min at 4° C., and the supernatant was stored as samples containing proteins from membrane-bound organelles.

### Echocardiography

Mice were anesthetized using isoflurane via a nasal cone, the chest was shaved, and the temperature of the mice was maintained at 37° C. The heart rate was continually monitored. Transthoracic echocardiography was performed using a VisualSonics Vevo 2100 system equipped with an MS400 transducer (FUJIFILM). LVEF and other indices of systolic function and thickness of LV walls were obtained from short-axis M-mode scans at the midventricular level, as indicated by the presence of papillary muscles. Apical four-chamber views were obtained for diastolic function measurements using pulsed-wave and tissue doppler imaging at the level of the mitral valve. During echocardiogram acquisition, isoflurane was maintained in 1.0-1.5% to maintain a heart rate in the range of 400-450 beats per min. All parameters were measured at least three times, and means are presented.

### Coronary Flow Reserve

Delineating images of the short axis of an aortic valve by echocardiography, a sample volume was put on the entry of the left coronary artery (LCA), and the blood flow rate was measured by doppler imaging. Data obtained from mice anesthetized by 1% isoflurane is regarded as baseline, and then isoflurane concentration was increased to 3% to obtain maximum coronary flow rate. CFR is calculated by dividing the maximum flow rate by the one at baseline.

### ALT, Cr and BUN Measurement in Serum

Serum samples were collected from WT and ΔE1HK1 mice at 40 weeks of age and analyzed with the Vetscan VS2 Chemistry Analyzer and Comprehensive Diagnostic Profile #500-0038 (Abaxis). Values of ALT, Cr and BUN between the groups were compared to assess the effect of HK1 localization on physiological functions of liver and kidneys.

### Exercise Exhaustion Test

The exercise exhaustion test was performed as previously described (Schiattarella et al., 2019). After three days of acclimatization to treadmill exercise, an exhaustion test was performed in the experimental groups of mice. Mice ran uphill (20°) on the treadmill (Columbus Instruments) starting at a warm-up speed of 5 m/min for 3 min after which speed was increased to 14 m/min for 2 min. Every subsequent 2 min, the speed was increased by 2 m/min until the mouse was exhausted. Exhaustion was defined as the inability of the mouse to return to running within 10 s of direct contact with an electric-stimulus grid. Running time was measured and running distance calculated.

### Grip Test

Mice were horizontally set in a straight position and allowed to hold a pull-bar assembly connected to an instrument to measure force generated. Experimenters held the mouse's tails and gently pulled the mice away from the instrument until they release the bar. The peak forces generated were measured three times, and average values were compared among experimental groups.

### Blood Pressure Measurement

Blood pressure (BP) was measured noninvasively in conscious mice using the tail-cuff method and a CODA instrument (Kent Scientific). Animals were placed in holders on a temperature-controlled platform (37° C.), and recordings were performed under steady-state conditions. Before testing, all mice were trained to become accustomed to short-term restraint. On the day of the experiments, BP was recorded for at least 30 cycles, and readings were averaged from at least 20 measurements.

### Lung Fluid Volume Measurement

Lungs were harvested and weighed immediately as wet lung weight. They were air dried for 60 hours, followed by dry lung measurement. Lung fluid volume was calculated by subtracting dry lung weight from wet lung weight.

### Immunogold Electron Microscopy

For immunogold electron microscopy (IEM), hearts of mice were perfused with 10 ml of PBS, followed by the perfusion of 30 ml of 4% PFA from the apex of them. The hearts were excised and incubated in 4% PFA overnight at 4° C. Small pieces of heart tissue were excised and incubated in the fixative overnight at 4° C. After post-fixation in 3% uranyl acetate samples were dehydrated in series of ethanol dilutions, embedded in LR White resin and polymerized for 48 hours in 50° C. Then ultrathin sections were made using Ultracut UC7 Ultramicrotome (Leica Microsystems) and incubated with HK1 antibody (CST, #2024) overnight at 4° C., followed by anti-rabbit secondary antibody conjugated with 12 nm gold particles (Jackson Immunoresearch). Sections were contrasted with 3% uranyl acetate and Reynolds's lead citrate. Samples were imaged using a FEI Tecnai™ G2 Spirit transmission electron microscope (EM) (FEI Company, Hillsboro, OR) operated at 80 kV. Images were captured by Eagle 4k HR 200 kV CCD camera.

### Measurement of Ca²⁺ Half Decay Time

Isolated CMs were incubated with 14 μM of the calcium indicator dye Cal520-AM (ATT Bioquest) for 25 min. Cells were added to the tissue bath on the stage of an inverted Zeiss LSM 510 (25×objective, N/A 1.2). The bath was perfused with Tyrode's solution (143 mM NaCl, 2.5 mM KCl, 25 mM NaHCO₃, 2 mM CaCl2), 2 mM MgCl2, 11 mM glucose, pH 7.4) and cells were paced at 1000, 500, 400, 300, and 200 ms cycle lengths (Gussak et al., 2020). Calcium transients were analyzed using custom software in MatLab 2015b, and WT and ΔE1HK1 mice were compared. Ca2+half decay time was measured as the time in which a peak Ca2+concentration in cytosol gets halved.

### Tube Formation Assay

The formation of tube networks was assessed as described before (Das et al., 2018). HUVECs and isolated ECs were seeded at 20,000 and 45,000cells per well in a 96-well plate coated with 75 mL Matrigel (Fisher Scientific) or Cultrex (R & D Systems) reduced growth factor basement membrane matrix. The cells were treated with EGM-2 medium containing tunicamycin (Sigma-Aldrich) or ST045849 (Sigma-Aldrich) wherever mentioned. Following an 18 hour-incubation, tube networks from each biological replicate were analyzed in at least three random fields by light microscopy. The number of branch points (junctions), segments and meshes, and total length of tubule networks (total length of branching) were quantified by Fiji software (Angiogenesis Analyzer).

### Proliferation Assay

Isolated ECs were seeded at 150,000 cells on 35 mm glass-bottom cell culture plates (Thermo Fisher Scientific) coated with 0.2% gelatin. After overnight incubation, 10 μM BrdU was added to the media, and cells were incubated for 4 hours. They were then fixated by 4% PFA incubation for 15 minutes, followed by 2N HCl incubation for 30 minutes, followed by blocking with 5% goat serum and 0.1% Triton X in PBS for 60 min, and incubated overnight with antibodies for BrdU antibody (5292, Cell Signaling Technology) in 1% BSA, 0.1% Triton X in PBS. Subsequent incubation with a secondary antibody and mounting media containing DAPI was performed as described in the method section of immunofluorescence. BrdU positive nuclei were counted and divided by the number of DAPI positive nuclei to calculate % BrdU positive.

### Transwell Migration Assay

Transwell migration assays were performed as described previously (Justus et al., 2014). Transwells with PET membrane with 8.0 m pores (353182, Corning) were coated with 20 g/ml of fibronectin (Sigma-Aldrich). Isolated ECs were seeded (100,000 cells) in the upper compartment of the transwell. EGM2 media was added to the bottom well to serve as a chemo-attractant for isolated ECs. ECs were allowed to migrate for 18 hours, after which cells were fixed, stained with 0.5% crystal violet with 2% Ethanol and the number of stained cells was quantified.

### Angiogenesis Assay in Mouse Neonatal Retina

Enucleated eye globes were obtained from neonatal WT and ΔE1HK1 mice at P5. Eyes were fixed (2% formaldehyde in PBS, pH7.5, 24h) before retinas were collected, blocked and permeabilized overnight (5% donkey serum, 2.5% bovine serum albumen, 0.5% Triton X100 in TBS pH 7.5). After blocking, retinas were incubated overnight in primary antibody diluted in additional blocking buffer (Goat anti-Pecam1, R&D Systems AF3628, 1:200), washed and then incubated in appropriate alexafluor-conjugated secondary antibody (Life Technologies). Retinas were then flat mounted on glass slides and imaged using a Nikon A1 confocal microscope equipped with a 10×objective. Stitched images were assembled using Nikon NIS Elements software, and sprouting front distance from the optic nerve head was measured using ImageJ Fiji.

### Oxygen Consumption and Extracellular Acidification Analysis

Isolated ECs were plated at 30,000 cells per well onto a 10 μg/mL fibronectin-coated 96-well Seahorse cell culture plate and grown for 18 hours in EGM-2 media. At least 18 hours before running a plate, the Seahorse sensor cartridge was incubated with Seahorse Calibrant solution according to the manufacturer's protocol in a 37° C., C02-free incubator. On the day of an assay, cells were washed and incubated with DMEM-based assay media. The sensor cartridge was fitted onto the cell culture plate, which was then placed into a 37° C., C02-free incubator for one hour. During the assay, which was run on the Seahorse XF96 Analyzer, the following inhibitors were injected sequentially, as is standard for the Mito Stress Test: oligomycin (1.5 μM), FCCP (1 M), and rotenone/antimycin (1 μM for each).

### ¹³Carbon Glucose Tracing and Steady-State Metabolomics

Cultured isolated ECs were treated with 5.5 mM of ¹³C6-glucose for 30 mins or 2 hours. Mass-spectrometry and metabolite identification were performed on 80% methanol and 20% ultrapure water extracted metabolites. Metabolomics services were performed by the Metabolomics Core Facility at Robert H. Lurie Comprehensive Cancer Center of Northwestern University. Samples were analyzed by High-Performance Liquid Chromatography and High-Resolution Mass Spectrometry and Tandem Mass Spectrometry (HPLC-MS/MS). The system consisted of a Thermo Q-Exactive in line with an electrospray source and an Ultimate3000 (Thermo) series HPLC consisting of a binary pump, degasser, and auto-sampler outfitted with an Xbridge Amide column (Waters; dimensions of 4.6 mm 3 100 mm and a 3.5 mm particle size). The mobile phase A contained 95% (vol/vol) water, 5% (vol/vol) acetonitrile, 20 mM ammonium hydroxide, 20 mM ammonium acetate, pH=9.0; B was 100% Acetonitrile. The gradient was as follows: 0 min, 15% A; 2.5 min, 30% A; 7 min, 43% A; 16 min, 62% A; 16.1-18 min, 75% A; 18-25 min, 15% A with a flow rate of 400 mL/min. The capillary of the ESI source was set to 275° C., with sheath gas at 45 arbitrary units, auxiliary gas at 5 arbitrary units, and the spray voltage at 4.0 kV. In positive/negative polarity switching mode, an m/z scan range from 70 to 850 was chosen, and MS1 data were collected at a resolution of 70,000. The automatic gain control (AGC) target was set at 1×106 and the maximum injection time was 200 ms. The top 5 precursor ions were subsequently fragmented, in a data-dependent manner, using the higher energy collisional dissociation (HCD) cell set to 30% normalized collision energy in MS2 at a resolution power of 17,500. The sample volumes of 10 ml were injected. Data acquisition and analysis were carried out by Xcalibur 4.0 software and Tracefinder 2.1 software, respectively (both from Thermo Fisher Scientific).

### Lectin Blot

Nitrocellulose membranes with blotted proteins were blocked with PBS containing 2% Tween 20 for 2 minutes at room temperature. They were rinsed with PBS twice, then incubated with 1 μg/ml of ConA-HRP (Sigma-Aldrich) in PBS containing 0.05% Tween 20, 1 mM CaCl2), 1 mM MnCl2, and 1 mM MgCl2 for 16 hours at room temperature avoiding exposure to the light. The presence of proteins conjugated with the lectin was visualized using Super Surgical Western Pico ECL substrate (Pierce). Quantification of the images was done using ImageJ (NIH).

### Generation of HK1 Lentivirus Gene Overexpression Constructs

Constructs to produce lentiviruses for overexpressing eGFP, FL-HK1 and Tr-HK1 were produced as described previously (De Jesus et al., 2022). To produce constructs for overexpressing FL-HK1-BioID2-HA and Tr-HK1-BioID2-HA, PCR fragments of BioID2-HA were obtained from MCS-BioID2-HA plasmid (#74224, Addgene) using primers with a 15-bp homology portion to the 3′-end of either FL-HK1 or Tr-HK1 just before the stop codon of the genes to facilitate recombination with In-Fusion (Takara Bio) reaction.

### Production of Lentiviruses and Stably Overexpressing HUVECs

To overexpress the gene of interest, HUVECs within passage number of 3 in a 35 mm-well were incubated with 25 l of concentrated viruses, 775 l of EGM2, and 0.8 l of polybrene (Millipore Sigma) for 8 hours, followed by addition of 1 ml of EGM2 media and incubation for another 16 hours. The media was replaced with fresh media without lentivirus and the cells were used for subsequent experiments 48 hours after the change of media (72 hours after the lentivirus incubation). To establish stably overexpressing HUVECs, 0.6 μg/ml of puromycin was added for 7 days to select the cells with the integration of the gene of interest into their genome. The successful selection was confirmed by immunoblot and immunofluorescence.

### Human Heart Samples

Procurement of human myocardial tissue was performed under protocols ethical regulations approved by Institutional Review Boards at the University of Pennsylvania and the Gift-of-Life Donor Program (Pennsylvania, USA) as previously described (Chen et al., 2018; Vite et al., 2022). Failing human hearts were procured at the time of orthotropic heart transplantation at the Hospital of University of Pennsylvania following informed consent from all participants. Non-failing (NF) hearts were obtained at the time of organ donation from cadaveric donors. In all cases, hearts were arrested in situ using ice-cold cardioplegia solution and transported on wet ice. Whole hearts and dissected left ventricle (LV) cavity were weighed to determine levels of hypertrophy. Transmural myocardial samples were dissected from the mid LV free wall below the papillary muscle. LV tissues for mass spectrometry and western blot were flash frozen in liquid nitrogen within 4 hours of explantation. Contractile parameters, including left ventricle ejection fraction (LVEF), were determined by echocardiography in patients. The heart samples were fixed in 4% PFA overnight and processed as described in the section of “Preparation of organ sections for histology and immunofluorescence” and “Immunofluorescence.”

### Co-Immunoprecipitation (Co-IP)

HUVEC cells stably overexpressing genes of interest were plated in 150 mm culture dishes and lysed using IP lysis buffer (25 mM Tris-HCl pH 7.4, 150 mM NaCl, 1 mM EDTA, 1% NP-40, and 5% glycerol) with protease arrest (Fisher Scientific). The lysate was centrifuged at 15,000×g and supernatant was collected in a fresh tube, followed by making the concentration of samples among the groups consistent. 25 μg of Protein G magnetic beads (Thermo Fisher Scientific) was washed with IP lysis buffer, then mixed with the sample, and 4 μg of the antibody of DDOST (14916-1-AP, Proteintech) or OGT (11576-2-AP, Proteintech) was added and incubated overnight at 4° C. on a rotator. The next day, beads were magnetically precipitated and washed 3×with IP lysis buffer. Bound protein from precipitated beads was eluted using sample buffer (4×Laemmli Sample Buffer [Bio-Rad], 10×NuPAGE sample reducing agent [Boston BioProducts] in ddH2O) and western blot analysis was performed.

### Biotinylated Protein Pulldown

Experiments to pulldown biotinylated proteins were performed as described previously (Sears et al., 2019). In brief, HUVEC stably overexpressing FL-HK1-BioID2-HA or Tr-HK1-BioID2-HA were seeded in 150 mm culture plates and treated with 50 μM of biotin (Sigma-Aldrich) in EGM2 for 18 hours. The cells were lysed using lysis buffer (8 M urea in 50 mM Tris pH7.4 with 1×protease arrest), followed by sonication and centrifugation at 15,000×g. The supernatant of the samples was collected, and the concentration of protein was made consistent among the samples. Preclearing of the samples with Gelatin-conjugated Sepharose 4B (GE Healthcare) was performed, then the supernatant was incubated with 50 μL of Streptavidin Sepharose High-Performance Beads (GE Healthcare) for 4 hours at 4° C. After the incubation, the complex of beads and proteins was washed with Lysis buffer on a rotator at room temperature for 8 minutes 3 times. To extract proteins from the complex, sample buffer (same formulation with IP experiments above) was added and the supernatant was collected as protein samples 10 minutes after incubation at 98° C. and used for subsequent experiments.

Analysis of scRNA-Seq Data

Expression levels of genes involved in glycolysis in CMs, FBs, and ECs in human embryo 9 weeks of gestation were analyzed using the scRNA-seq data available in the National Center for Biotechnology Information Gene Expression Omnibus with the accession ID GSE106118 (Cui et al., 2019). Relative TPM were obtained by dividing each value of TPM by the mean value of TPM in CMs, and they were plotted in the form of a violin plot.

### Analysis of Proteomics Data

Proteomics data in a previous study was obtained from MassIVE (Accession number: MSV000088901) (De Jesus et al., 2022). The numbers of detected fragments of peptides corresponding to the amino acid sequence of RPN1, RPN2, and DDOST in each group were analyzed.

### Synthesis and Administration of 5S-GlcNHex to Mice

5S-GlcNHex (WuXi AppTec Co., Ltd, China) was synthesized as described previously (Liu et al., 2018). 10, 20, 30, 40 or 50 mg/kg of the chemical was dissolved in 100 μL of PBS and injected into the intraperitoneal cavities of mice. Isolated ECs were collected 0, 18, 24, 40, and 48 hours after injection, followed by purification of protein samples. Subsequently, levels of 0-GlcNAcylation of proteins in the samples were analyzed by immunoblot experiments. For the assessment of the effect of 5S-GlcNHex on the development of HFpEF, C57BL/6J mice were treated with HFD_L-NAME for 5 weeks, followed by the injection of 20 or 30 mg/kg 5S-GlcNHex into the intraperitoneal cavities every 2 days for 10 weeks, and then echocardiography was performed. Similarly, 50 mg/kg of 5S-GlcNHex was administered to mice every 2 days for 3 weeks following 5 weeks of HFD_L-NAME treatment for the assessment of cardiac function.

### Statistics

Data are presented as mean±SEM. Single comparisons were assessed by an unpaired Student t-test. Statistical significance was assessed with ANOVA, with post hoc Tukey's test for multiple group comparison. For a comparison of non-parametric data, the Mann-Whitney test was performed. A P-value less than 0.05 was considered statistically significant. The analysis was conducted using GraphPad Prism9 software.

### Example 1—HK1 is highly expressed in ECs compared to CMs and FBs

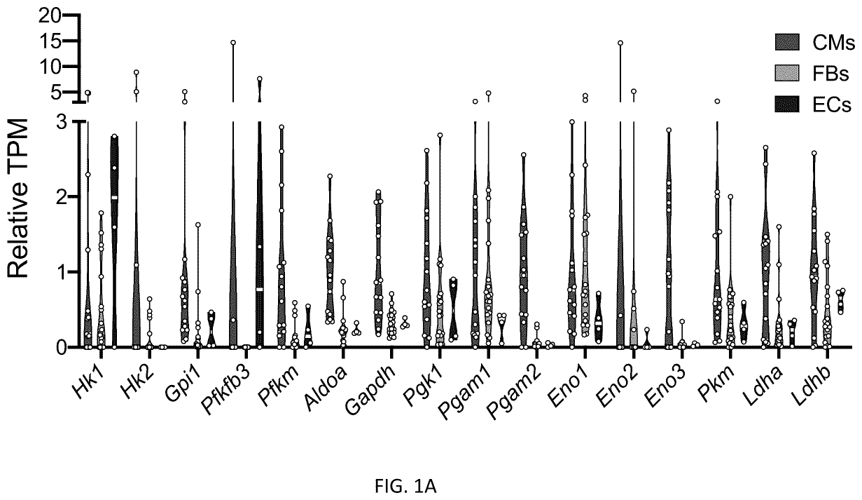

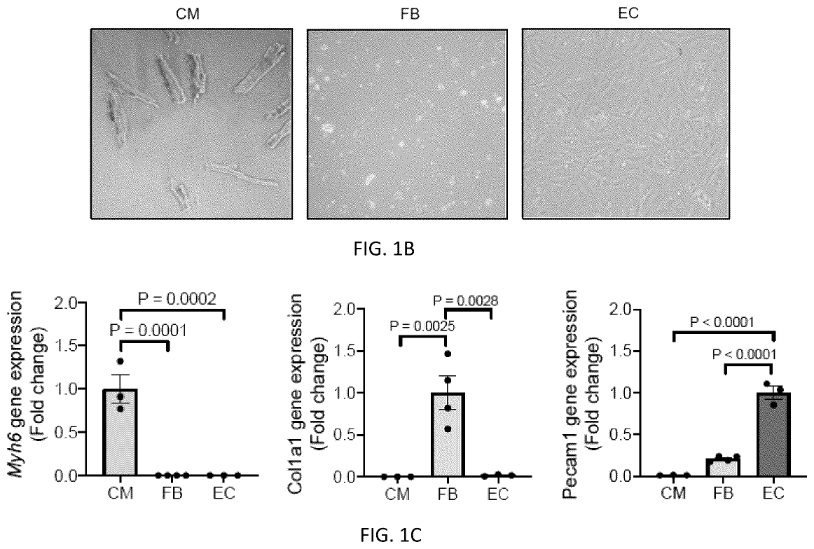

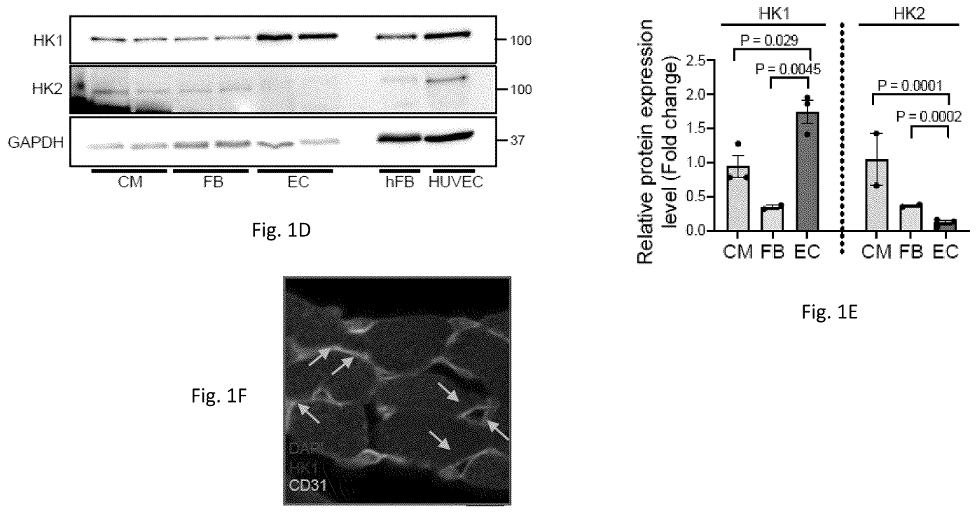

Previous studies have shown a critical role for EC dysfunction in the development of HFpEF (Paulus and Tschope, 2013), and that ECs are highly dependent on glycolysis for energy production and angiogenesis (De Bock et al., 2013).Thus, the studies were first focused on how changes in glycolytic enzymes of ECs contribute to the pathogenesis of HFpEF. A deposited single-cell RNA sequencing (scRNA-seq) data encompassing genes involved in glycolysis across various cardiac resident cell types were analyzed. Violin-plot of relative transcripts per million (TPM) among CMs, FBs, and ECs in scRNA-seq data derived from human hearts displayed that Hk1 mRNA expression predominates in ECs compared to the other cell types (FIG. 1A). To corroborate these findings, CMs, FBs, and ECs from the hearts of C57BL/6J mice were isolated and protein levels of HK1 and HK2 were quantified (FIG. 1B). qPCR data of cell type-specific markers confirmed the purity of the isolated cells, except for minimal contamination of ECs in FB fraction (FIG. 1C). Consistently, protein levels of HK1 were significantly higher in ECs than in the other cell types (FIGS. 1D and 1E). Furthermore, increased protein levels of HK1 in human umbilical vein endothelial cells (HUVEC) were also observed compared to human FBs (FIG. 1D). Immunofluorescence studies using an HK1 antibody in mouse heart sections further supported the findings by illustrating strong fluorescent signals of HK1 localized within capillaries (FIG. 1F). These data emphasize the elevation of HK1 expression in ECs relative to other cardiac cell types, suggesting an important role for HK1 in the physiological functions of ECs.

### Example 2—HK1 is dislocated from mitochondria in ECs in hearts of HFpEF mouse model

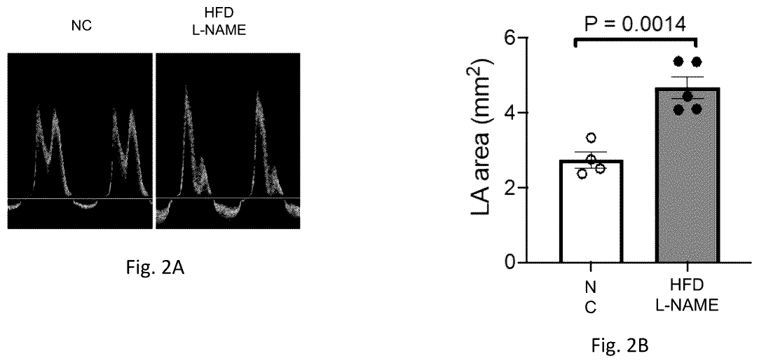

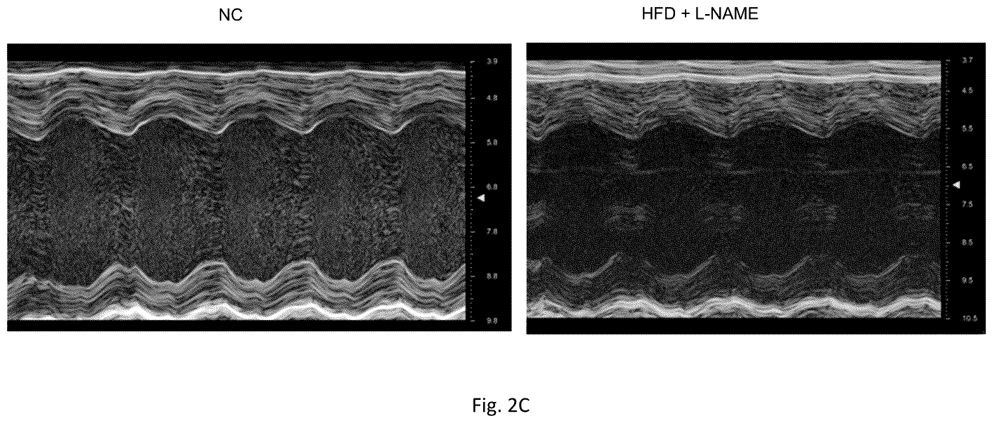

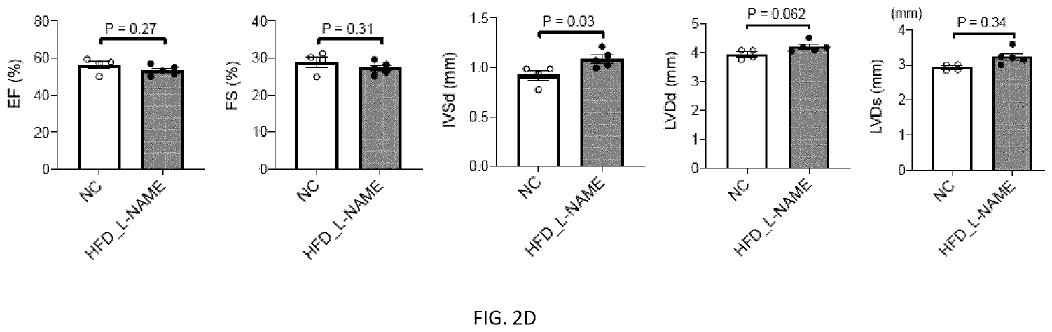

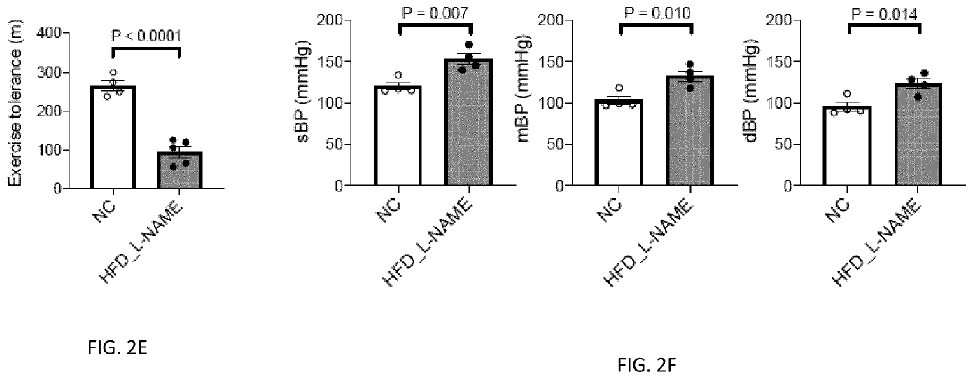

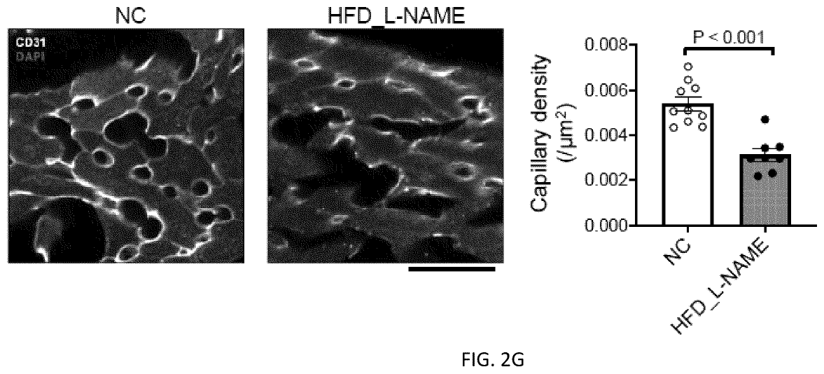

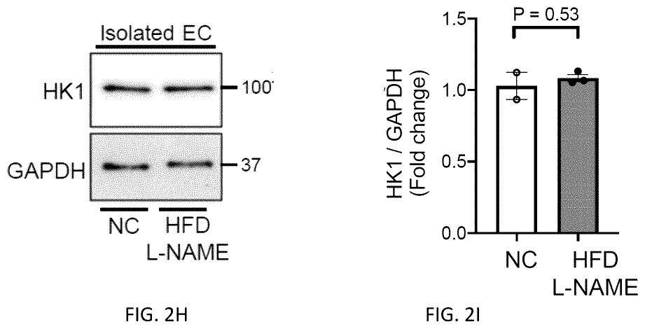

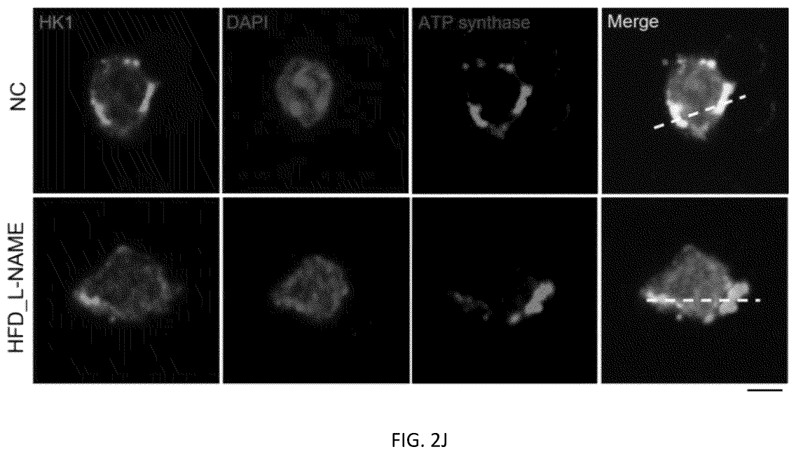

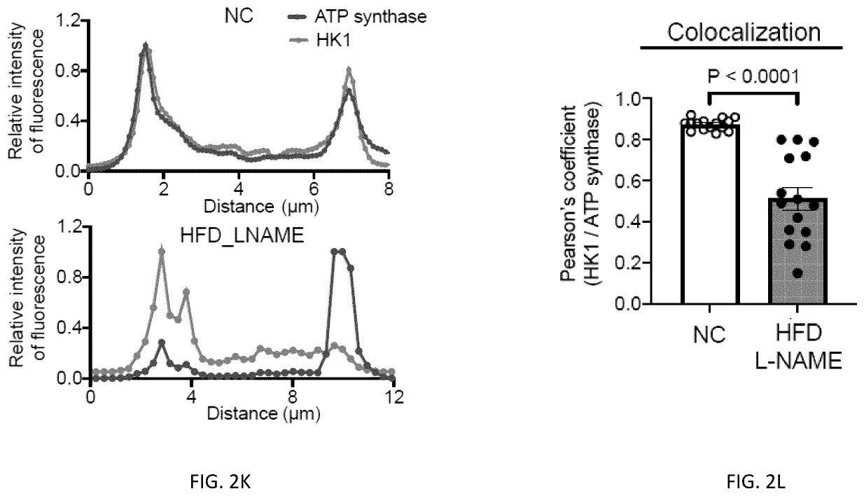

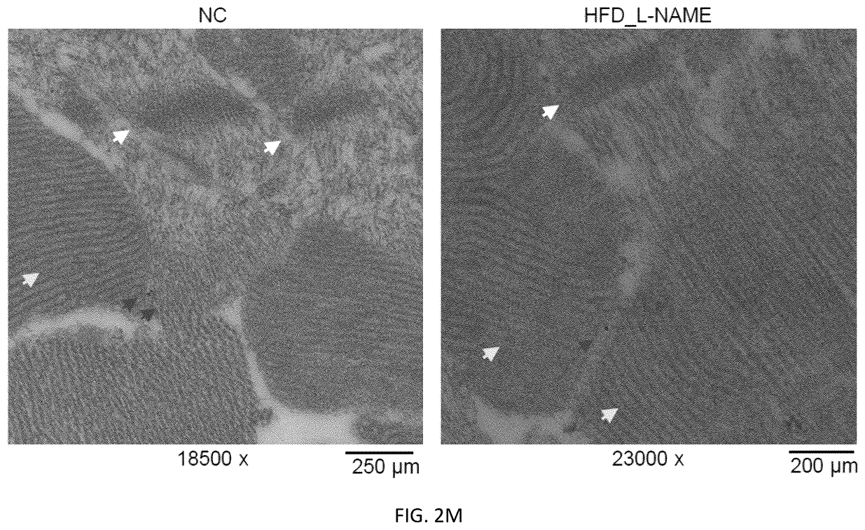

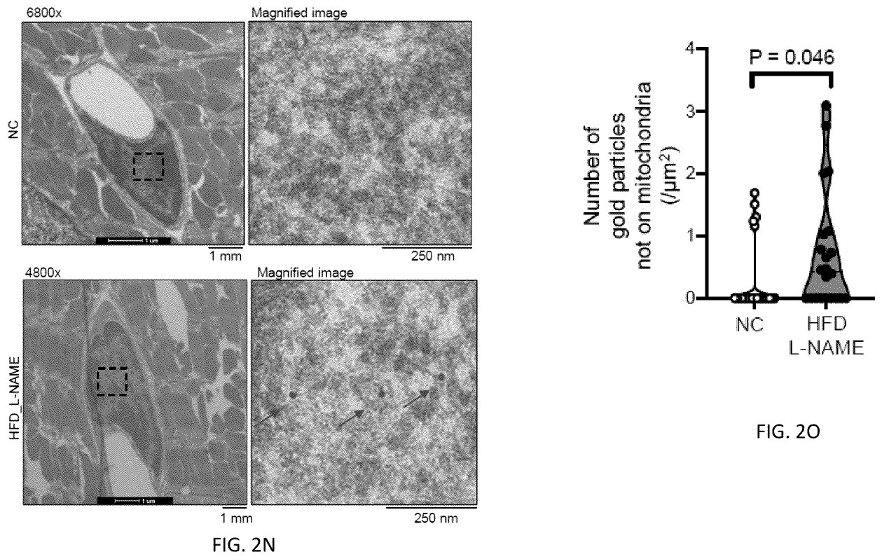

Given that EC dysfunction is a major driver of HFpEF, the expression level of HK1 in ECs in a murine model of HFpEF was evaluated (Schiattarella et al., 2019). Mice treated with a high-fat diet (HFD) and No-Nitro-L-arginine methyl ester (L-NAME) for 15 weeks exhibited significantly elevated E/A and E/e' ratios (FIG. 2A), along with increased left atrial (LA) area (FIG. 2B) compared to the normal chow (NC) group, but no change in systolic function and left ventricular (LV) structure (FIGS. 2C and 2D), indicating that these mice develop impaired LV relaxation and elevated end-diastolic LV pressure with preserved ejection fraction (EF). HFD_LNAME treated mice displayed reduced exercise tolerance and elevated blood pressure (BP) relative to the normal chow (NC) group (FIGS. 2E and 2F), which were consistent with a previous report (Schiattarella et al., 2019). Additionally, hearts from HFD_L-NAME treated mice showed a decreased capillary density compared to the NC group FIG. 2G). Together, these data confirm the successful induction of HFpEF in mice through the administration of HFD_LNAME treatment.

Protein expression of HK1 in ECs from hearts of HFpEF mouse model were next assessed, and no differences were noted (FIGS. 2H and 2I). Since HK1 subcellular localization exerts profound effects on cellular physiology and progression of some disorders (De Jesus et al., 2022; Rossi et al., 2020), the localization of HK1 within ECs in the hearts of the HFpEF mouse model was characterized. In freshly isolated ECs from NC mice, clear colocalization of HK1 with mitochondria were observed, whereas distinct non-colocalized HK1 signals were detected in mice subjected to HFD_L-NAME (FIGS. 2J-2L). This observation strongly suggests the HK1 dislocation from mitochondria within ECs of HFpEF hearts. To gain further insights into the subcellular localization of HK1 within ECs in HFpEF hearts, immunogold electron microscopy (IEM) with an anti-HK1 antibody was performed. The efficacy of IEM was confirmed by detecting gold particles on the OMM in CMs (FIG. 2M). Subsequently, analyses revealed higher number of gold particles not present on mitochondria (i.e., in the cytoplasm and nucleus) within ECs in mice treated with HFD_L-NAME (FIGS. 2N and 2O). Thus, although HK1 expression levels remained unaltered, its subcellular localization shifted from a predominantly mitochondrial-bound to a cytosolic and nuclear localization in HFpEF.

### Example 3—ΔE1HK1 mice spontaneously develop HFpEF with aging

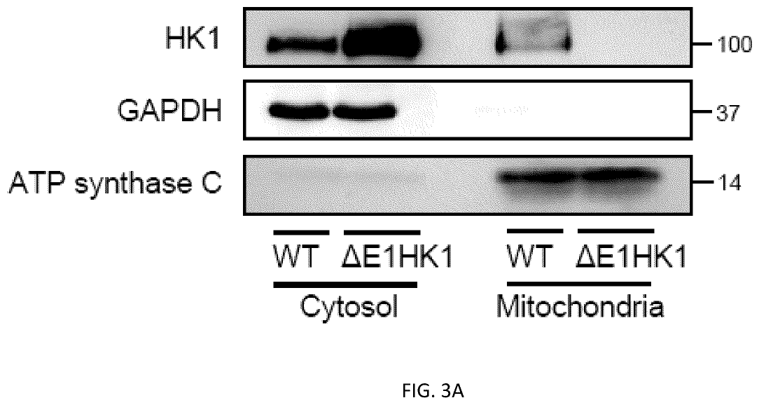

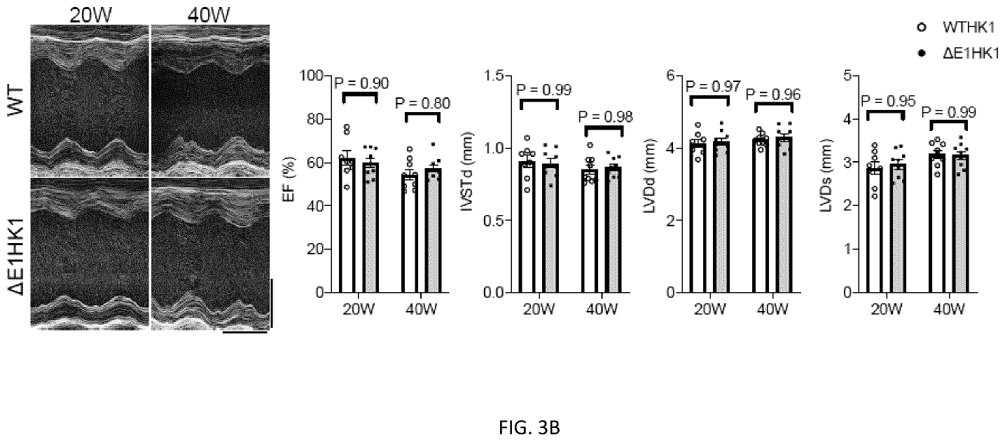

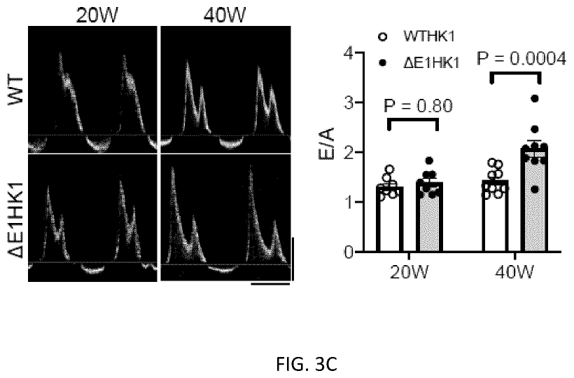

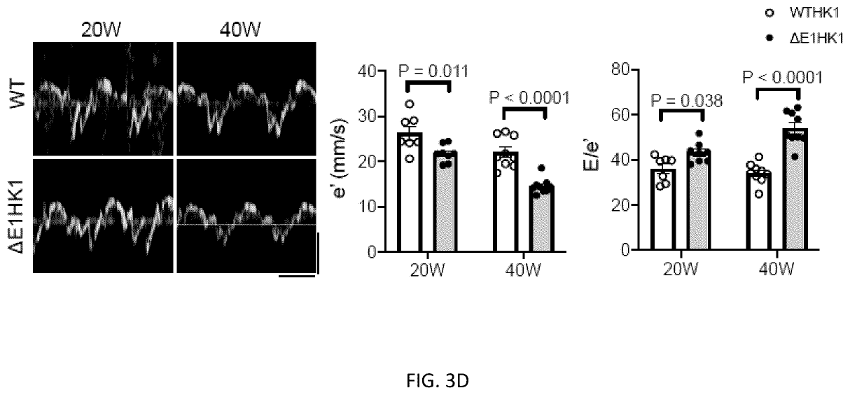

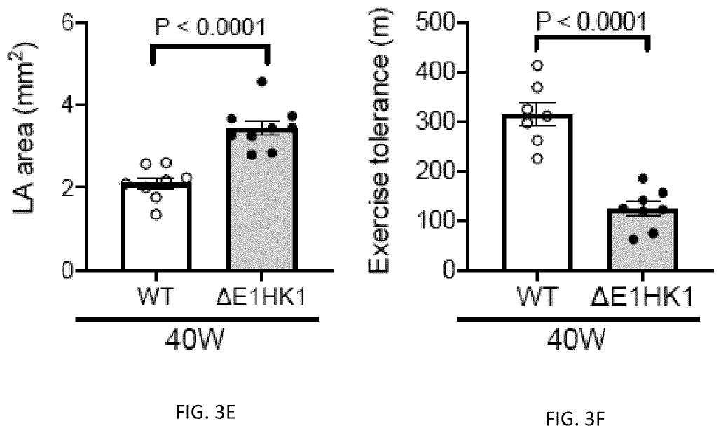

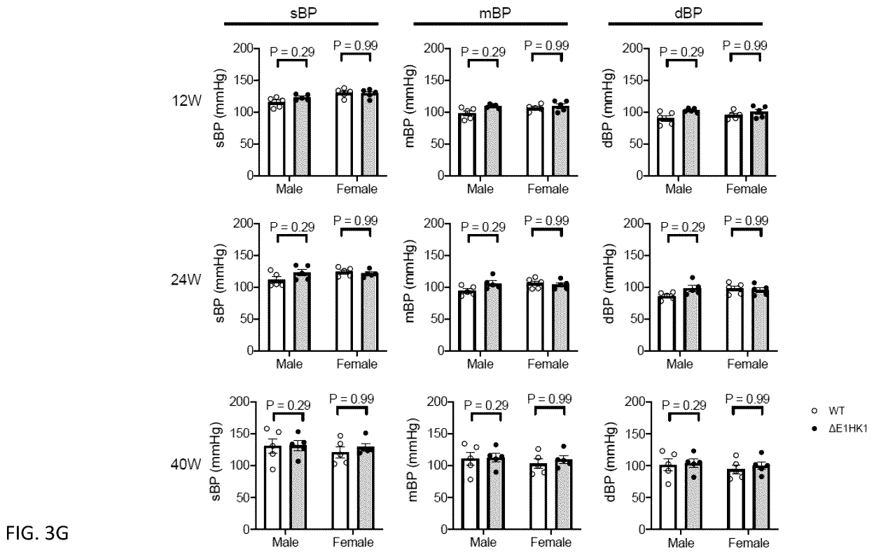

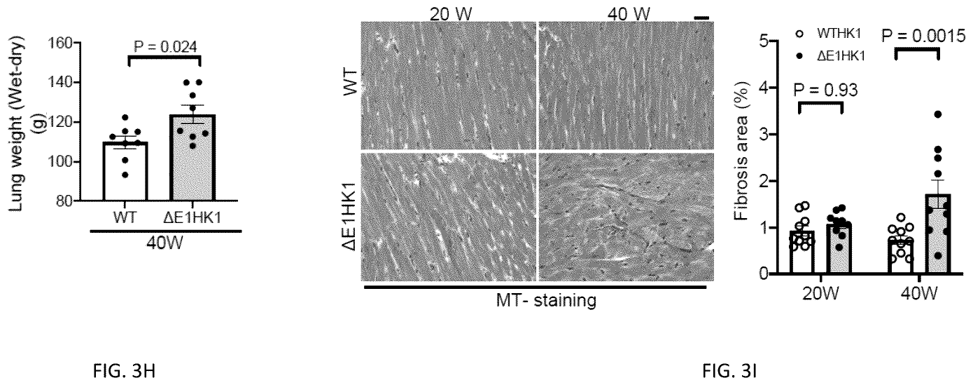

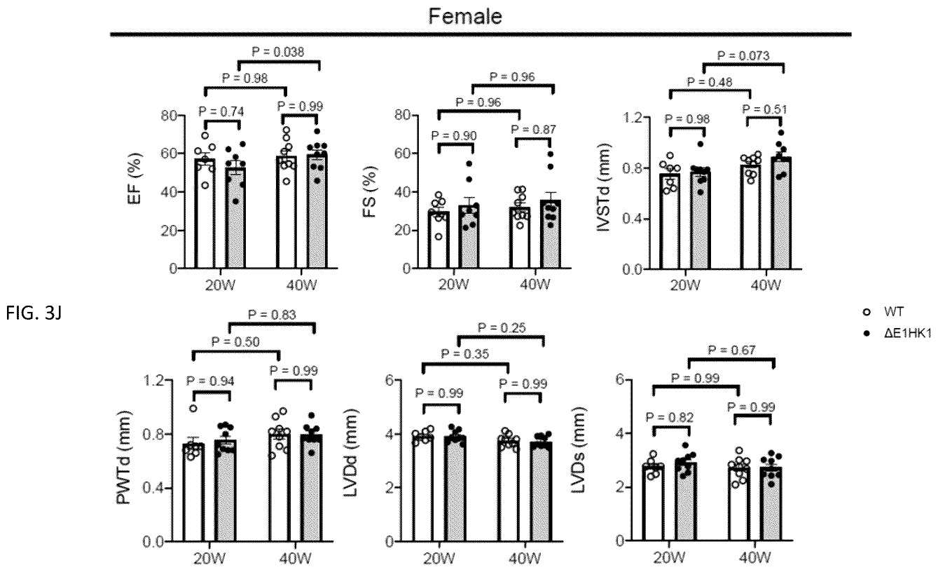

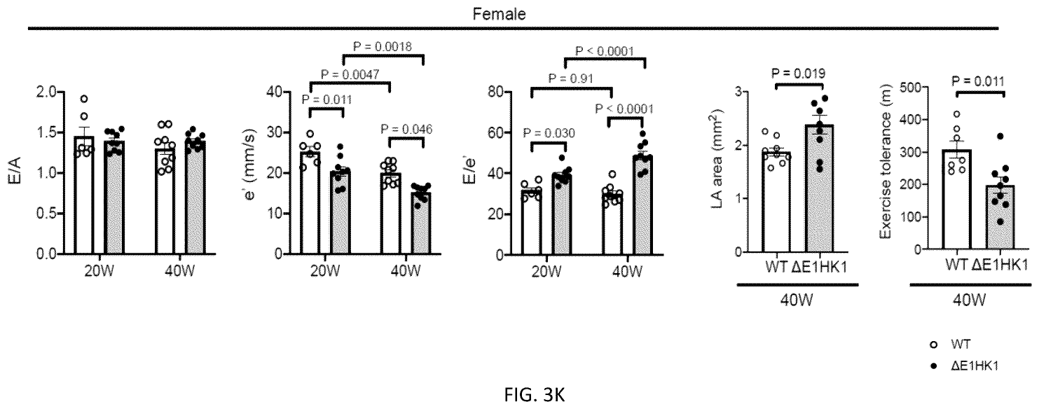

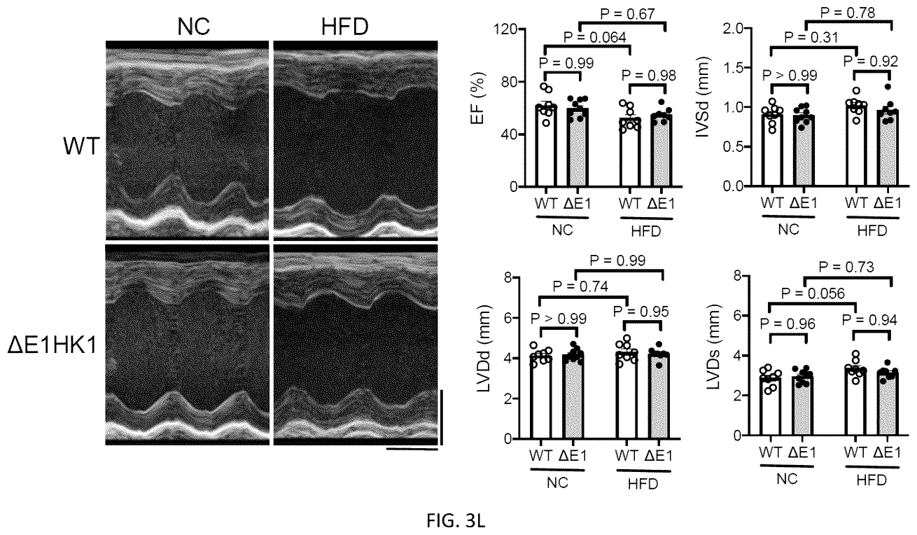

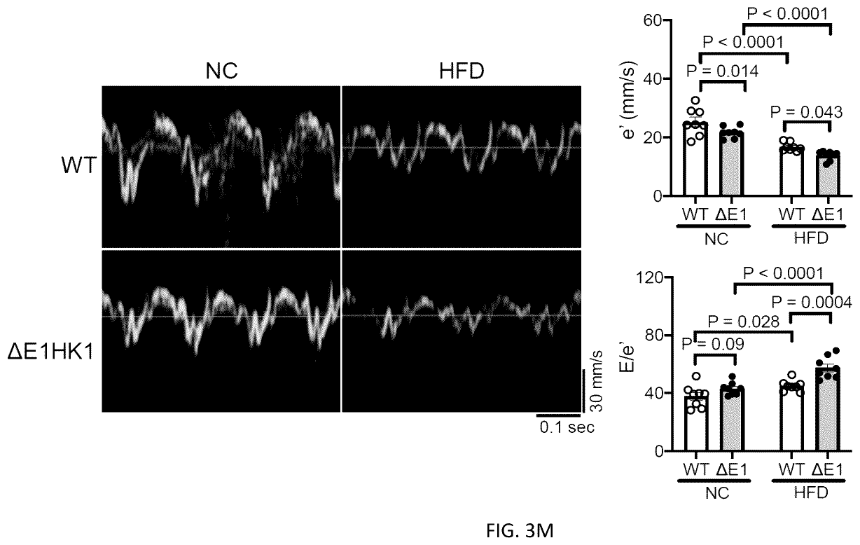

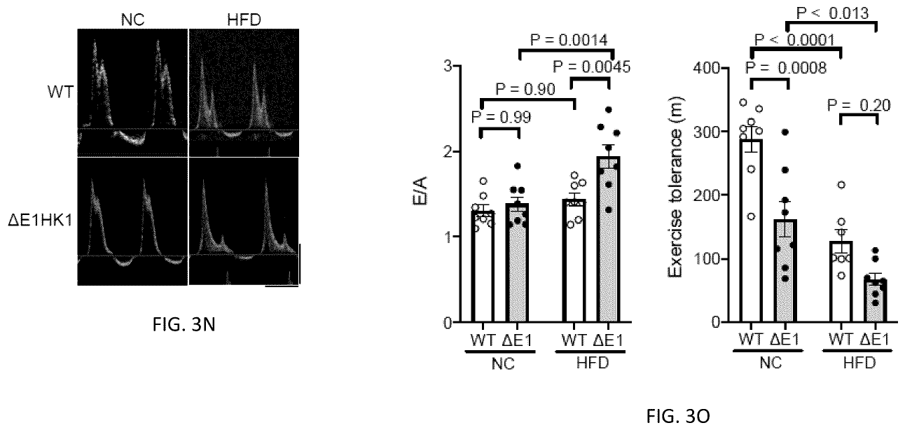

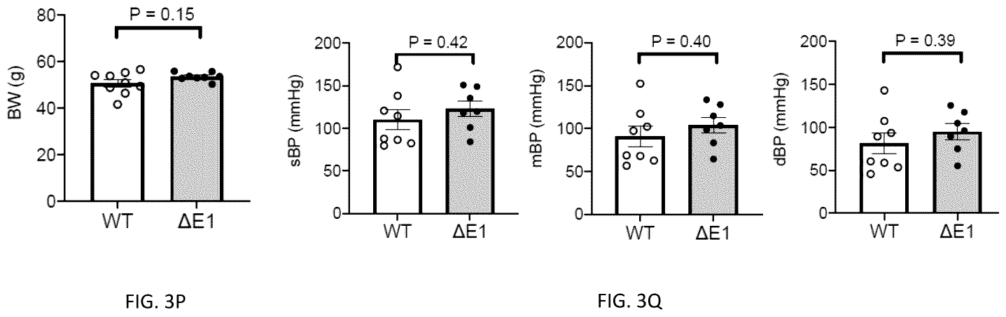

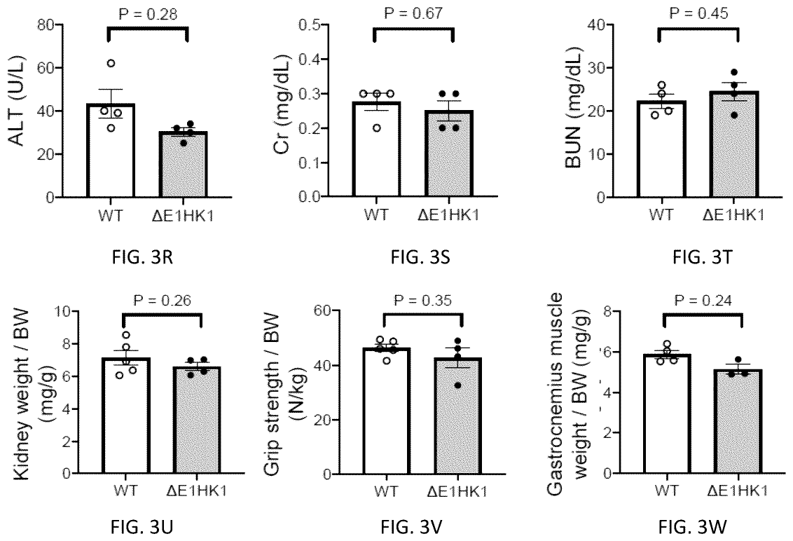

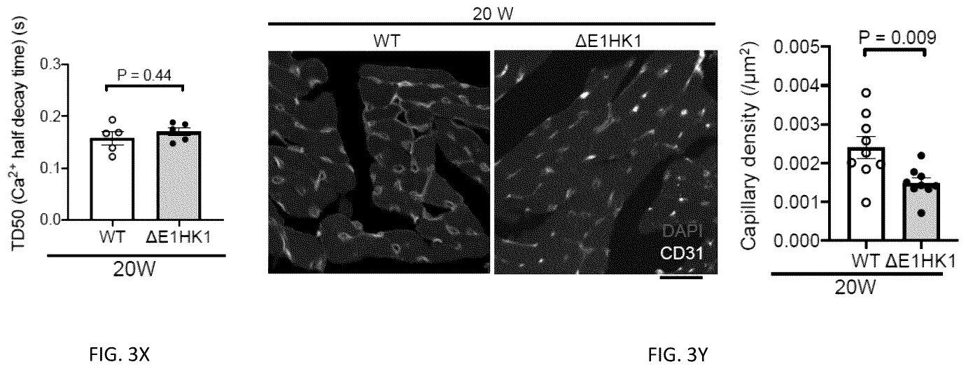

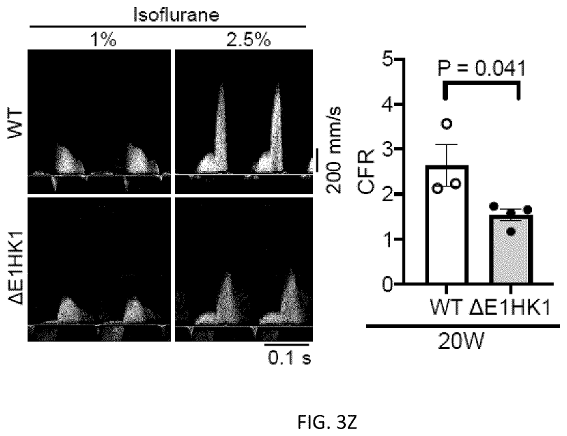

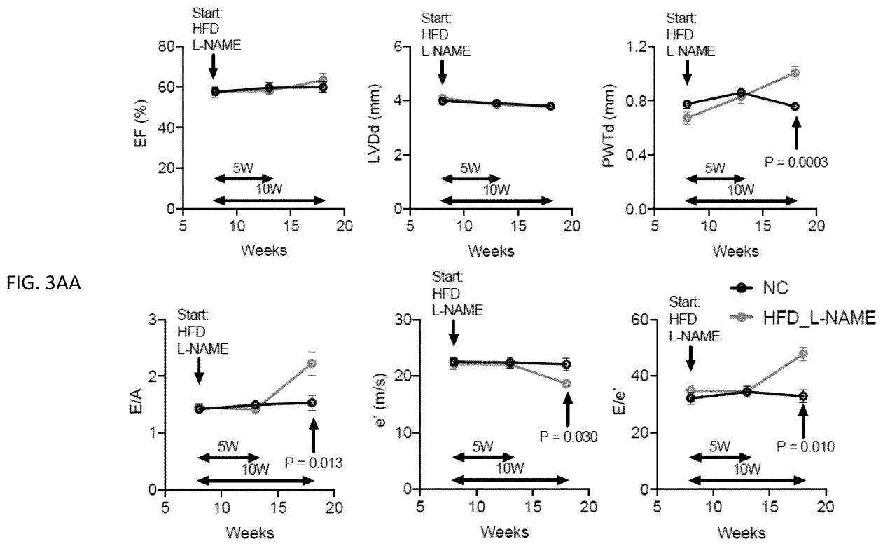

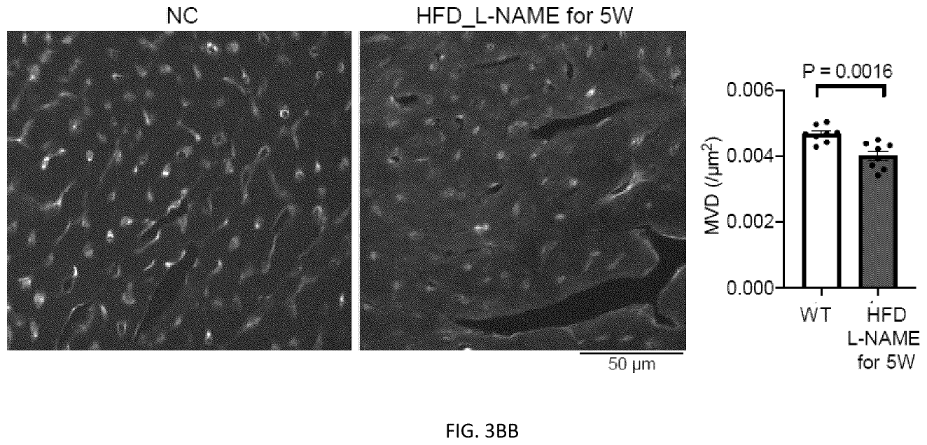

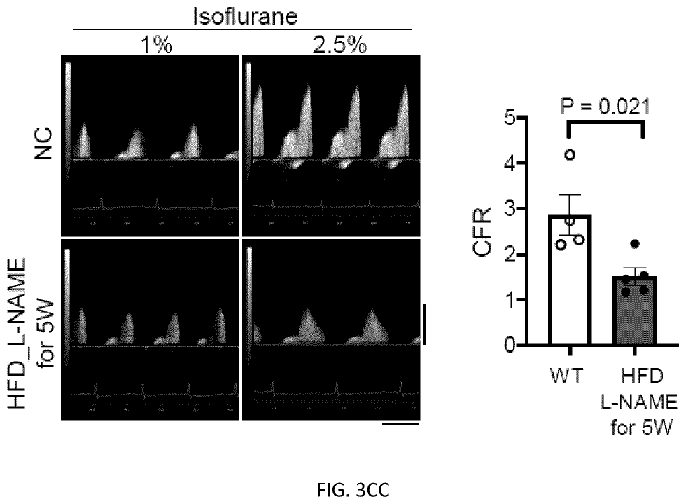

To better define the consequences of HK1 dislocation from mitochondria on the progression of HfpEF, a mouse model with the MBD of the endogenous HK1 gene replaced with a FLAG sequence (ΔE1HK1 mice) was employed, described previously (De Jesus et al., 2022). As expected, HK1 was predominantly localized in the non-mitochondrial fraction of the ΔE1HK1 hearts (FIG. 3A).

Echocardiographic analysis of the hearts of these mice at 40 weeks of age showed no significant alterations in the systolic function or LV structure (FIG. 3B). However, there were significant reductions in e' and concurrent increase in E/A and E/e' (FIGS. 3C and 3D), along with an enlarged LA area, diminished exercise tolerance (FIGS. 3E and 3F), and no substantial changes in BP (FIG. 3G). Furthermore, ΔE1HK1 mice also displayed an increased fluid volume in their lungs (FIG. 311). Additionally, histological assessments revealed a higher percentage of fibrosis in the hearts of ΔE1HK1 mice at 40 weeks of age (FIG. 3I). Notably, while HFD_LNAME treatment leads to HFpEF only in male mice (Schiattarella et al., 2019), significant decreases in e' and exercise tolerance was observed, as well as an increase in E/e' and LA area in female ΔE1HK1 mice, with no significant change in E/A ratio (FIG. 3J-3K). These results indicate that dislocation of HK1 from mitochondria is associated with an overt HFpEF phenotype in male mice and manifests most features of diastolic dysfunction in female mice.

WT and ΔE1HK1 mice at 8 weeks of age were then treated with HFD for 20 weeks. Strikingly, the ΔE1HK1 mice subjected to HFD displayed heightened E/e' and E/A ratios, and reduced exercise tolerance despite comparable EF, body weight (BW) and BP when compared to WT mice (FIGS. 3L-3Q). Thus, in addition to developing HFpEF with advanced age, ΔE1HK1 mice are more prone to HFpEF induction in response to HFD.

Systemic glucose tolerance and insulin sensitivity in the mice had been previously reported and were found to be comparable between WT and ΔE1HK1 mice (De Jesus et al., 2022). Further characterization of the ΔE1HK1 mice revealed that aspartate aminotransferase (ALT), creatinine (Cr) and blood urea nitrogen (BUN) levels in serum and kidney weight were similar between the two groups (FIGS. 3R-3U), suggesting their liver and renal functions remain unaltered. Similar strength in a grip test and the weight of gastrocnemius muscle were also observed, indicating skeletal muscle function is preserved in ΔE1HK1 mice (FIGS. 3V and 3W).

To investigate the underlying mechanism by which ΔE1HK1 develop HFpEF, isolated CM relaxation was assessed by measuring the half-time decay of Ca2+transient and microvascular density in the hearts of ΔE1HK1 mice at the age of 20 weeks, a time point preceding the overt HFpEF phenotype. While Ca2+decay times in isolated CMs were comparable between WT and ΔE1HK1 mice (FIG. 3X), a reduction in microvascular density within the ΔE1HK1 hearts was observed (FIG. 3Y), suggesting the occurrence of microvascular rarefaction before the development of HFpEF in these mice. Additionally, diminished coronary flow reserve (CFR) in 20-week-old ΔE1HK1 mice was noted (FIG. 3Z). Studies were next extended to C57BL6/J mice treated with a 5-week regimen of HFD_L-NAME, a period during which they do not develop HFpEF yet (FIG. 3AA). These mice exhibited decreased microvascular density and CFR (FIGS. 3BB and 3CC), mirroring the observation made by ΔE1HK1 mice. These findings collectively suggest that EC dysfunction likely precedes cardiac dysfunction and may propel the pathogenesis of HFpEF.

### Example 4—Isolated ECs from Hearts of ΔE1HK1 Mice Display Reduced Angiogenic Potential

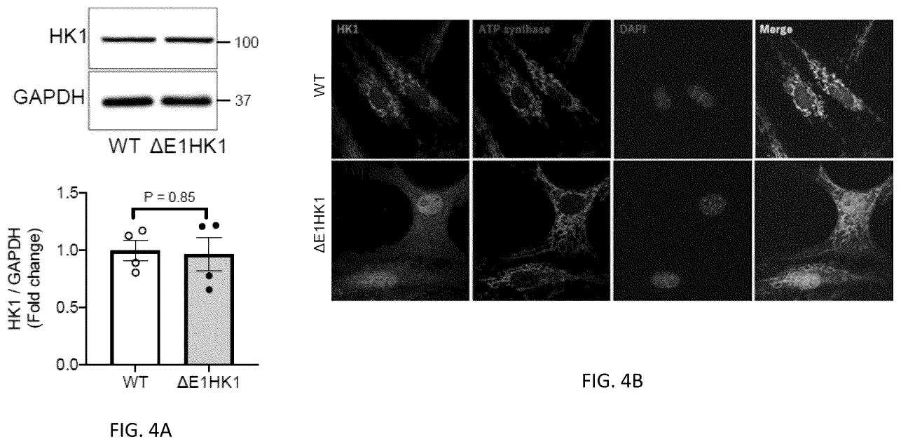

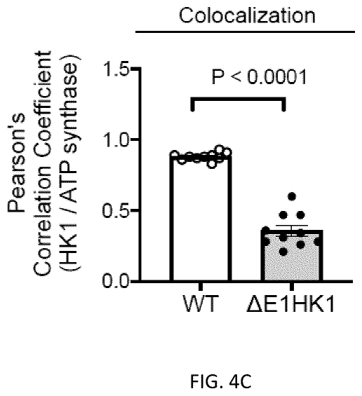

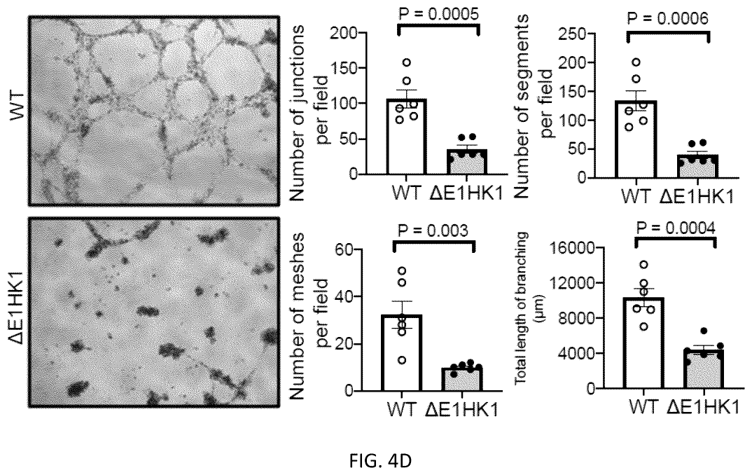

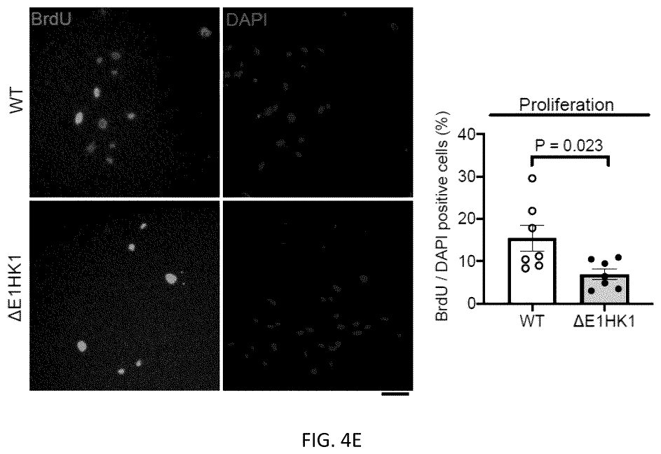

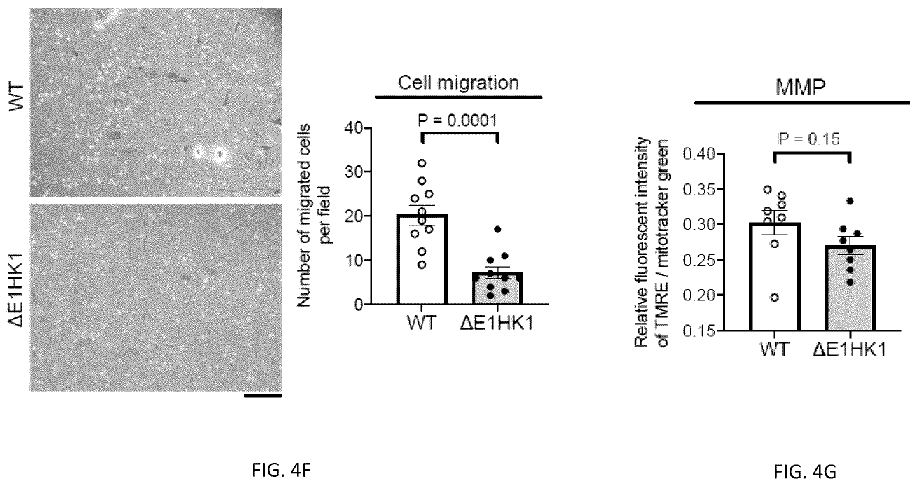

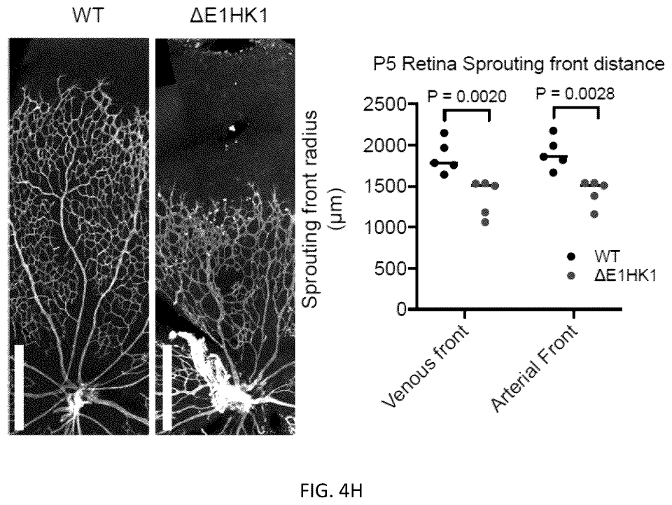

Next, the angiogenic potential of ECs from the hearts of ΔE1HK1 mice were studied. While protein levels of HK1 remained unchanged (FIG. 4A), HK1 was dislocated from mitochondria in ECs isolated from ΔE1HK1 hearts (FIGS. 4B and 4C). Tube formation assays demonstrated that ECs originating from ΔE1HK1 mice displayed a decreased number of branching points, segments of meshes formed by tubes, and less total length of branching in comparison to ECs from WT hearts (FIG. 4D). Furthermore, less proliferation and motility in the isolated ECs from ΔE1HK1 mice was also observed, as assessed by bromodeoxyuridine (BrdU) incorporation and transwell migration assays (FIGS. 4E and 4F), whereas mitochondrial membrane potential (MMP) was preserved in EC from ΔE1HK1 mice (FIG. 4G). Notably, sprouting front radius in retinas from P5 neonates of ΔE1HK1 mice was also significantly reduced compared to WT (FIG. 411). Together, these results suggest that ECs of ΔE1HK1 mice exhibit diminished angiogenic potential, likely due to defects in proliferation and motility independent of MMP.

### Example 5—Levels of Metabolites in HBP are Reduced in Isolated ECs from Hearts of ΔE1HK1 Mice

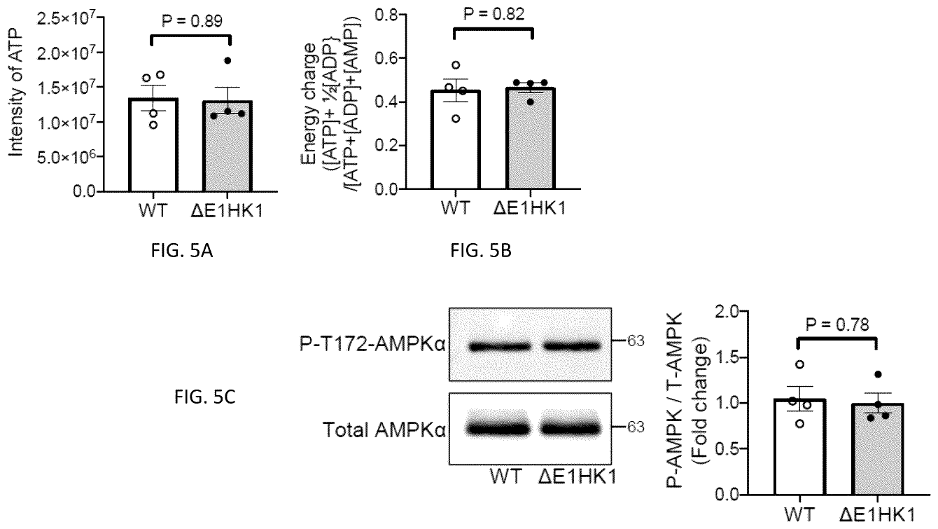

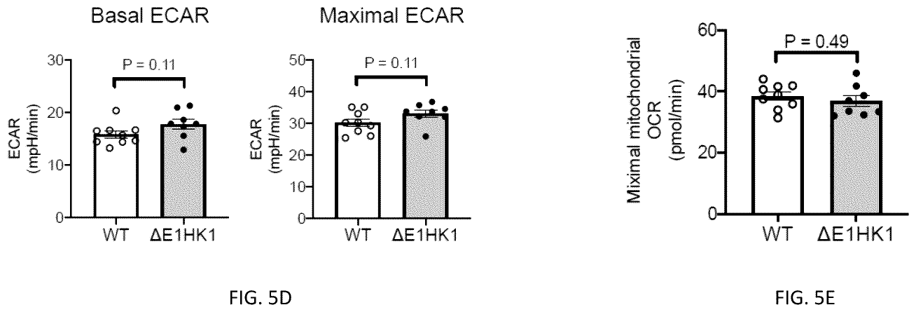

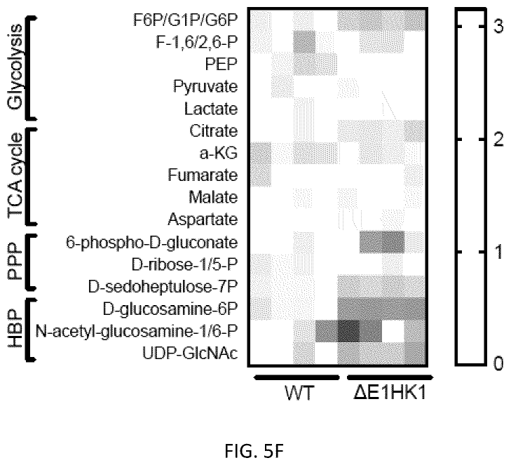

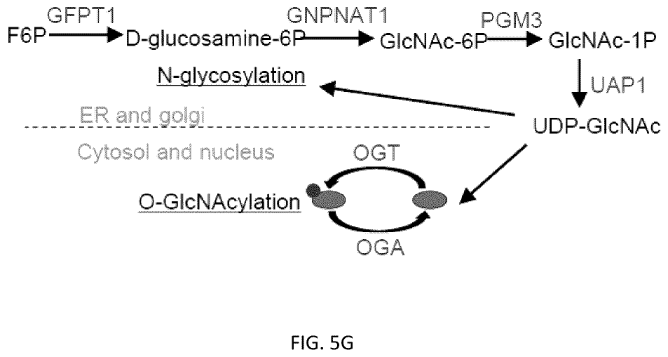

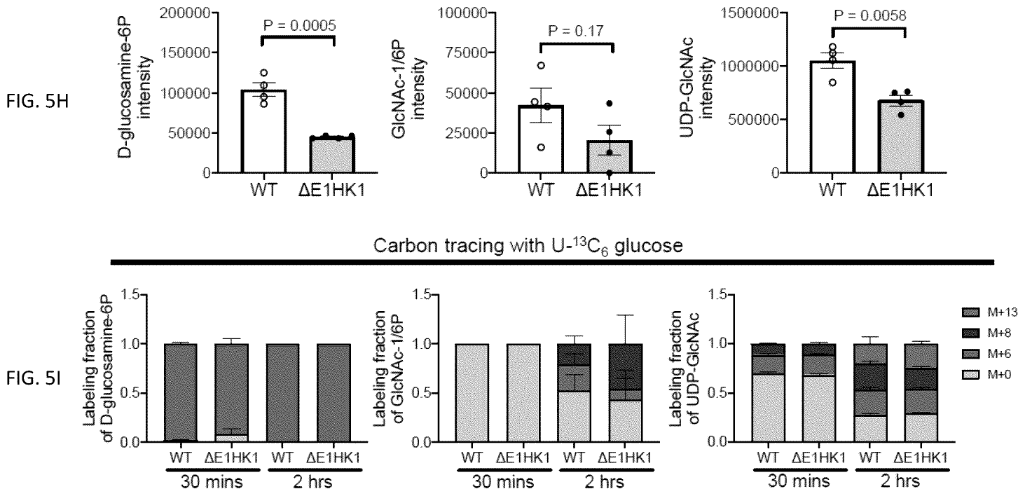

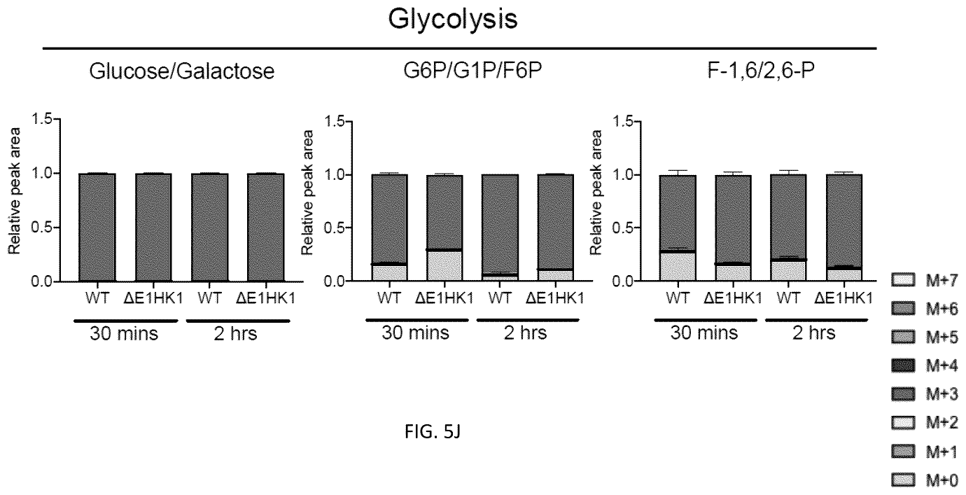

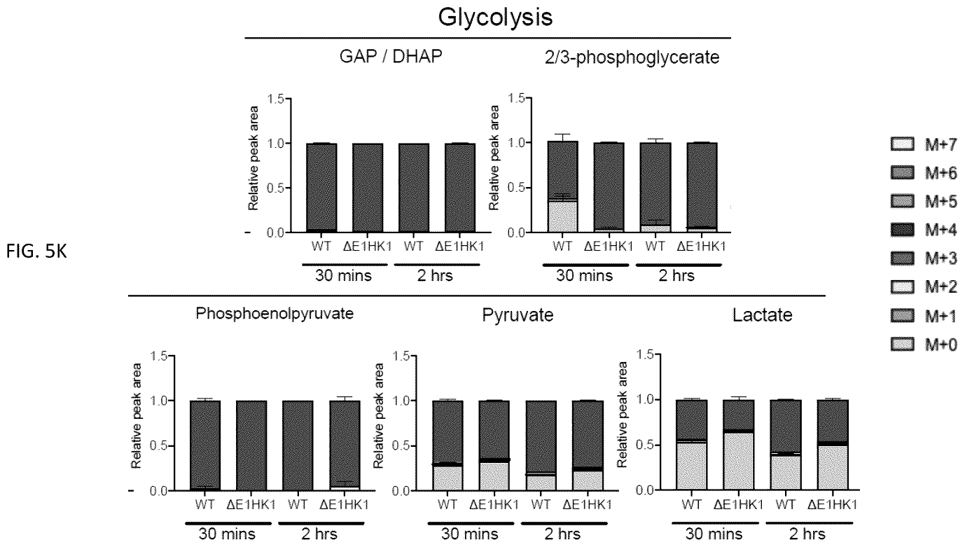

To understand the mechanisms underpinning the reduction of angiogenesis in ECs from ΔE1HK1 mice, energy state of ECs from WT and ΔE1HK1 mice were studied. No discernible differences in total cellular ATP levels, energy charge calculated by the levels of adenine nucleotides, and the ratio of protein levels of the phosphorylated form of AMPKα to total AMPKα were found (FIGS. 5A-C). Additionally, extracellular acidification rate (ECAR) and oxygen consumption rate (OCR) were similar among the groups, suggesting that glycolytic flux and the activity of the TCA cycle and electron transport chain in mitochondria were comparable between isolated ECs from WT and ΔE1HK1 mice (FIGS. 5D and 5E).

Metabolomic profiling of isolated ECs from WT and ΔE1HK1 mice revealed consistent reduction in the levels of HBP metabolites in ECs from ΔE1HK1 mice, while the levels of metabolites in PPP were slightly increased (FIGS. 5F-511). Most metabolites in the glycolysis and TCA cycle showed similar levels between the two groups. To explore the mechanisms underlying the decrease in HBP metabolites within ECs from ΔE1HK1 mice, isotope tracing metabolomics using U-¹³C6-glucose were performed, which displayed similar labeling of HBP metabolites in the ECs from both WT and ΔE1HK1 mice (FIGS. 51-5N). Additionally, the mRNA and protein levels of genes involved in HBP were not altered (FIGS. 50 and 5P). The carbon-tracing studies and the analysis of genes in HBP collectively suggest that the flux of HBP itself is not altered in ECs from WT and ΔE1HK1 mice. However, the decrease in the HBP metabolites is likely attributed to changes downstream of HBP, including processes related to protein N-glycosylation and 0-GlcNAcylation.

### Example 6—Dislocation of HK1 in ECs Causes a Reduction in Protein N-Glycosylation

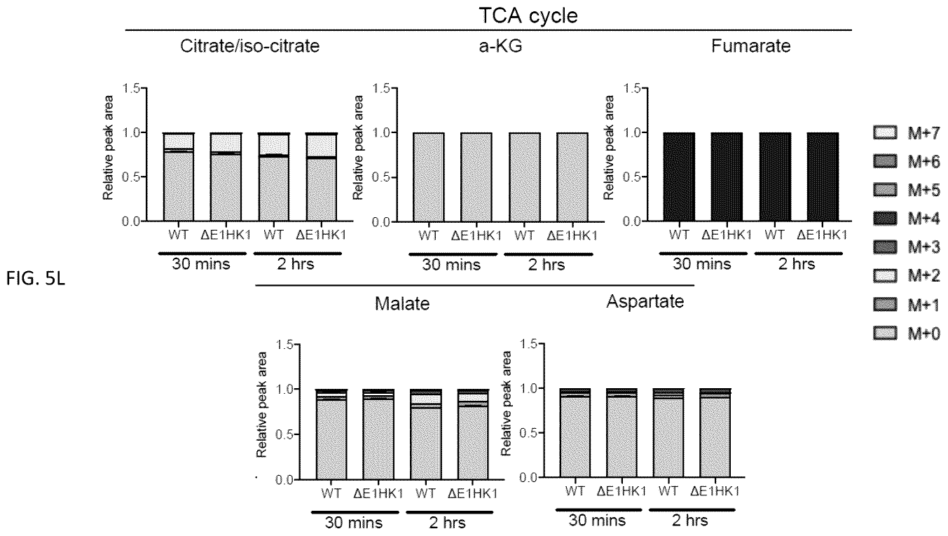

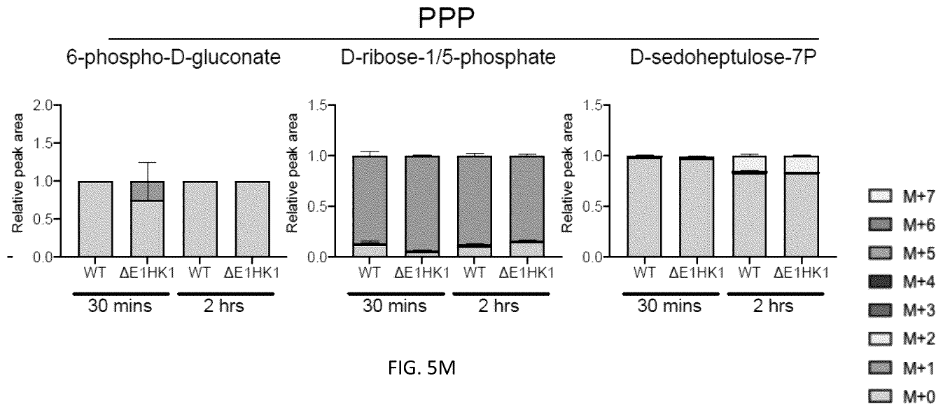

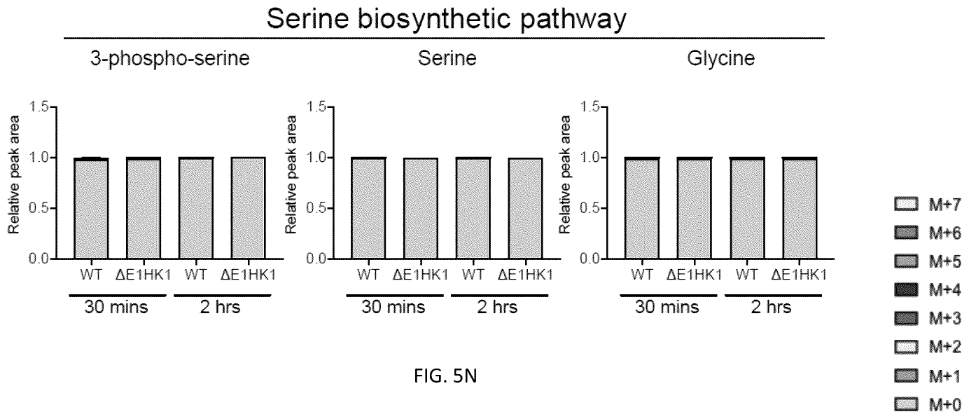

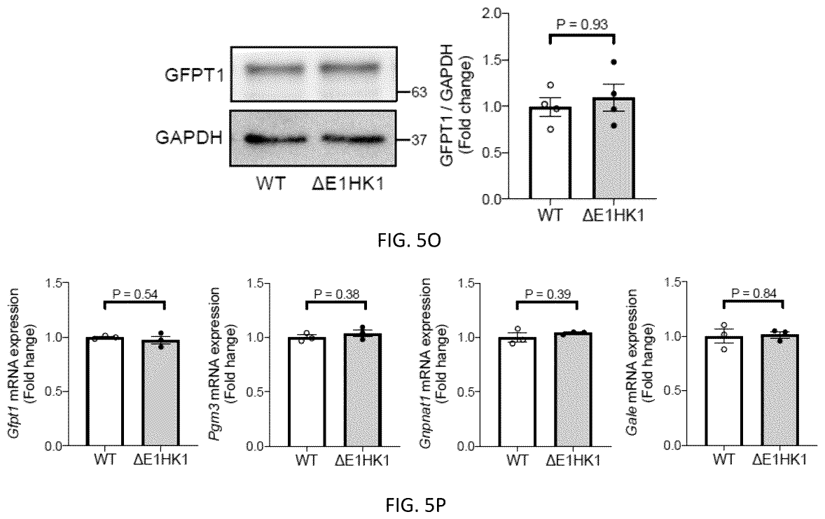

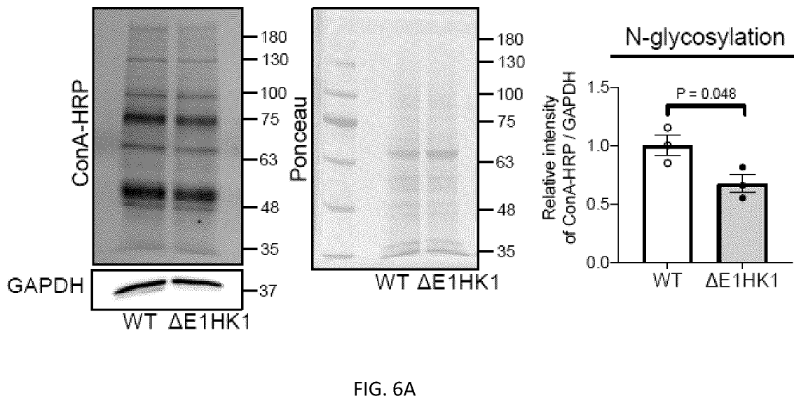

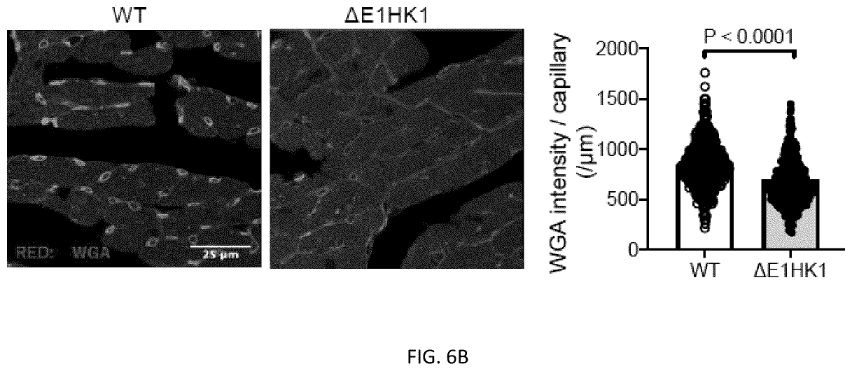

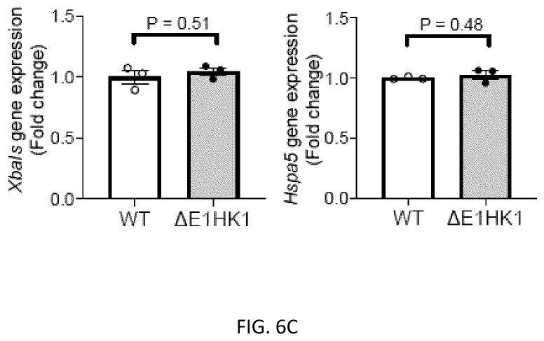

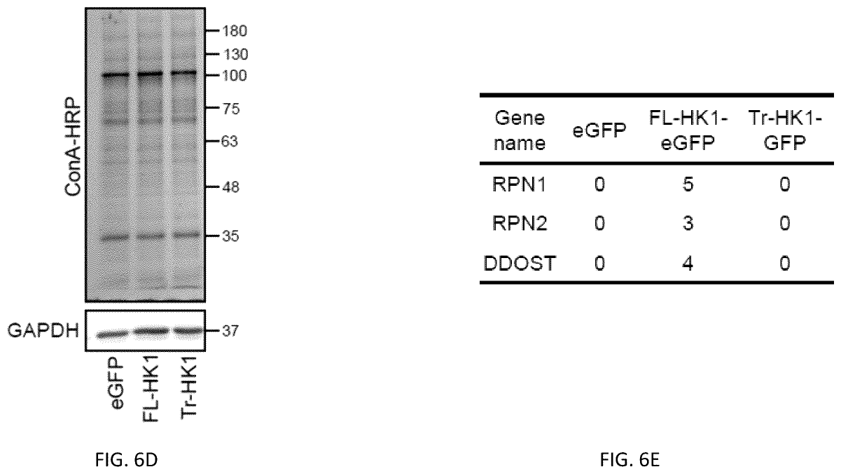

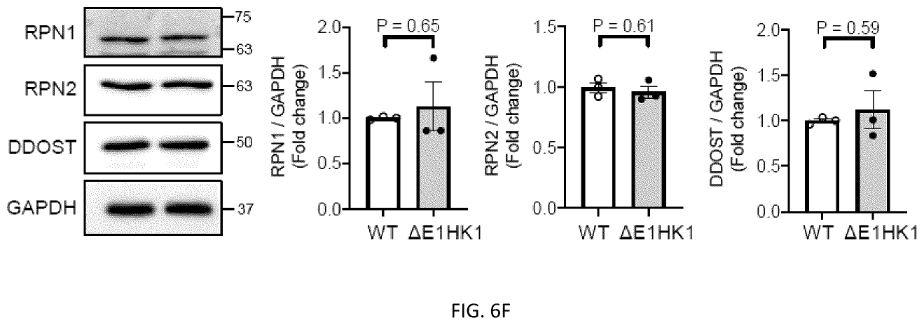

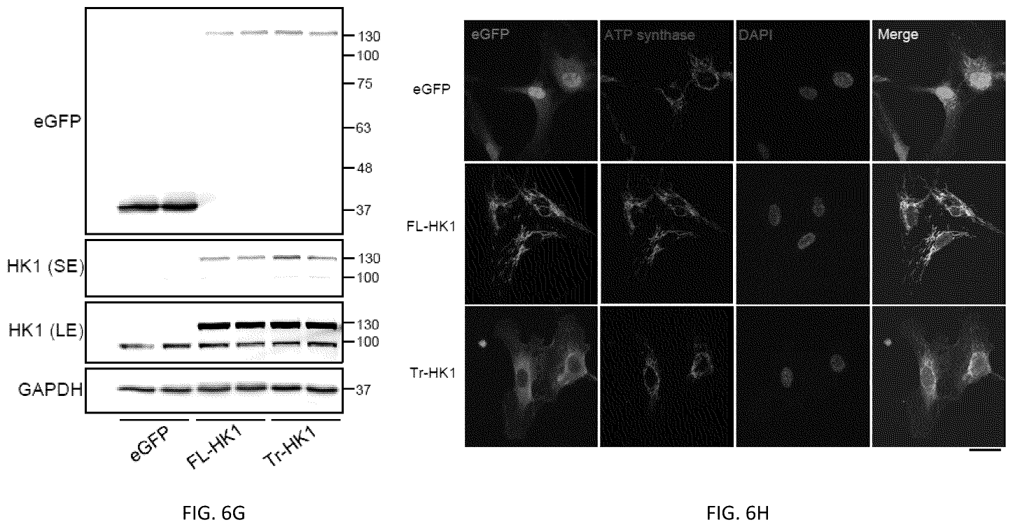

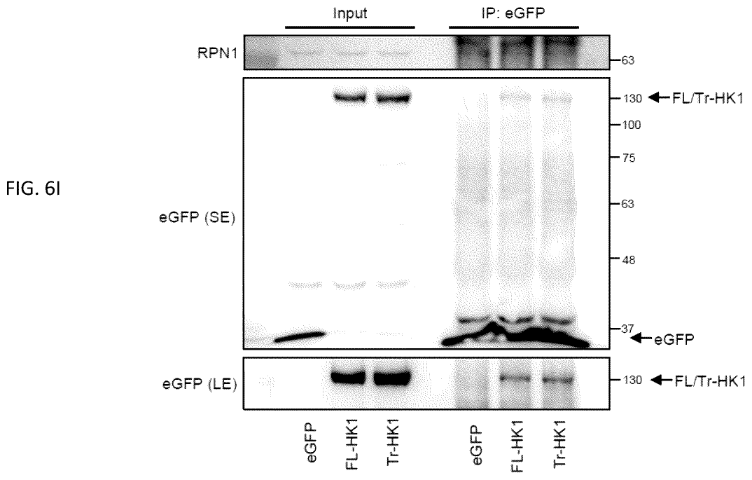

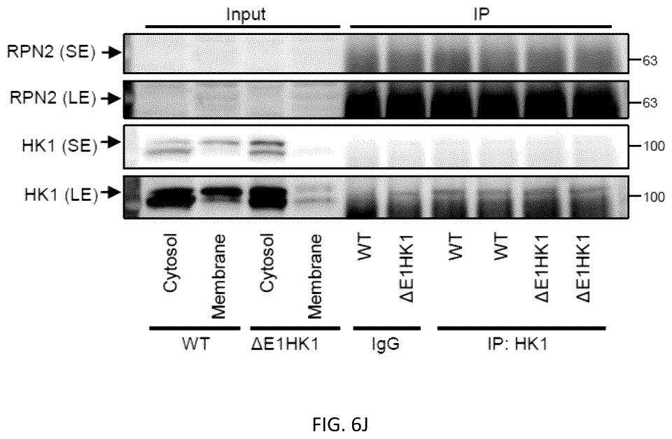

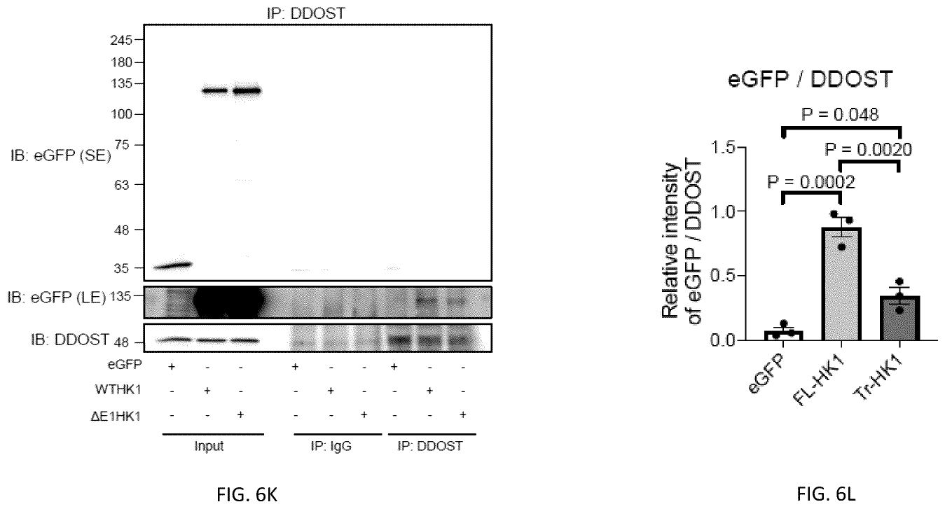

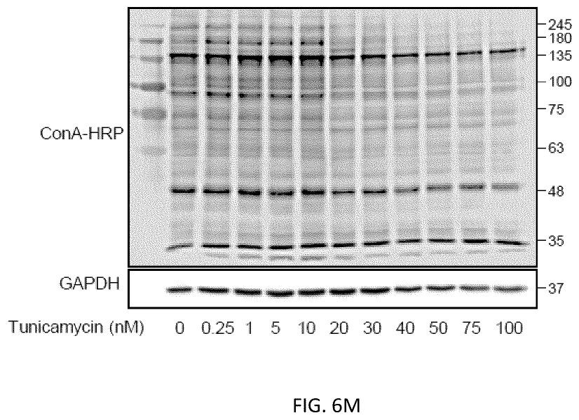

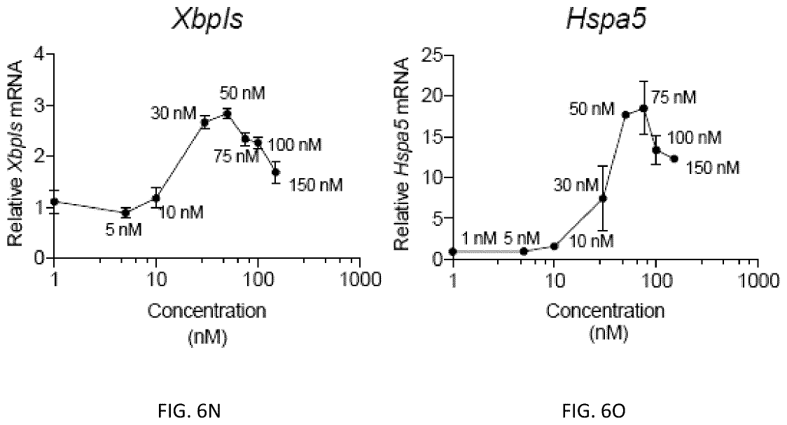

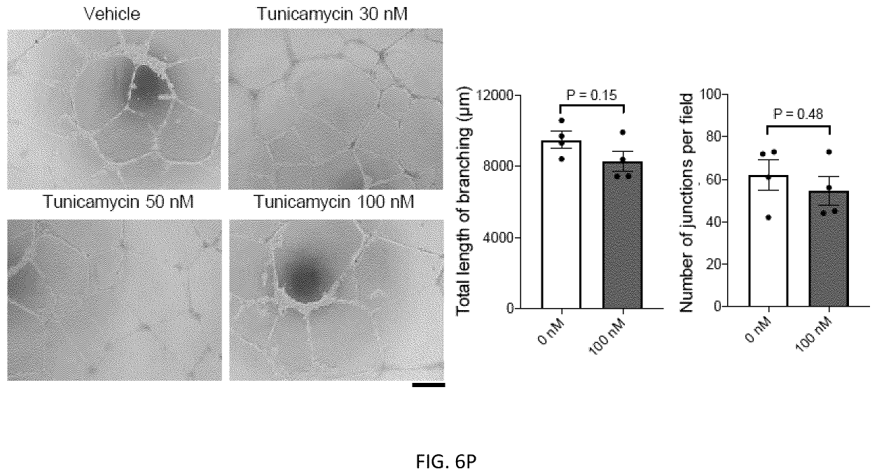

The initial investigation focused on protein N-glycosylation, which is one of the primary consequences of HBP. N-glycosylation of proteins was significantly reduced in ECs from ΔE1HK1 mice, as evidenced by lectin blot analysis using Concanavalin A (ConA)-horseradish peroxidase (HRP) (FIG. 6A). Additionally, the assessment of glycan levels on membrane proteins based on wheat germ agglutinin (WGA) fluorescence in capillaries further confirmed the reduction of N-glycosylated proteins (FIG. 6B). Despite the reduction in N-glycosylated proteins, the mRNA levels of genes involved in unfolded protein response (UPR) remained unchanged between ECs from WT and ΔE1HK1 mice (FIG. 6C), suggesting the proper protein folding is preserved in spite of decreased protein N-glycosylation. Either full length HK1 (FL-HK1) or truncated HK1 lacking the MBD (Tr-HK1) conjugated with eGFP was also overexpressed in HUVECs, but no effect on protein N-glycosylation was noted (FIG. 6D), likely because additional mitochondrial HK1 expression does not have a capacity to increase N-glycosylation.

An analysis of proteomic studies on immunoprecipitated samples with an anti-GFP antibody in HepG2 cells overexpressing either FL-HK1 or Tr-HK1 (De Jesus et al., 2022) indicated that ribophorin 1 (RPN1), RPN2, and dolichyl-diphosphooligosaccharide-protein glycosyltransferase non-catalytic subunit (DDOST), subunits of oligosaccharyl transferase, are possible binding partners of FL-HK1, but not Tr-HK1 (FIG. 6E). The inventors first confirmed that there were no significant differences in the protein levels of RPN1, RPN2, and DDOST in the ECs from WT and ΔE1HK1 mice (FIG. 6F). Subsequently, co-immunoprecipitation (co-IP) experiments using HUVECs stably overexpressing eGFP, FL-HIK1, and Tr-HK1 (FIGS. 6G and 6H) showed no clear evidence of binding of HK1 and RPN1 or RPN2 (FIGS. 6I and 6J). In contrast, DDOST was detected to interact mostly with FL-HK1, and not Tr-HK1, in HUVECs (FIGS. 6K and 6L). These results indicate that mitochondrial localization of HK1 is crucial for the interaction between HK1 and DDOST, and that the reduced N-glycosylation observed in the ECs of ΔE1HK1 mice is likely due to the lack of this interaction.

Given the importance of mitochondria and endoplasmic reticulum (ER) interaction in physiological cellular processes and glucose metabolism (Rieusset, 2018; Szymanski et al., 2017), whether the interaction between HK1 and DDOST and reduced N-glycosylation has an effect on the angiogenic potential of ECs was assessed. Tube formation of HUVECs with tunicamycin, an agent that inhibits the N-glycosylation machinery (Jin and Chung, 2018), was first assessed by conducting preliminary dose-curve experiments, and confirmed reduced protein N-glycosylation at 20 nM of tunicamycin with the plateau at 50 nM (FIG. 6M). The mRNA levels of genes related to UPR acted in the same manner as observed in N-glycosylation experiments (FIGS. 6N and 6O). Based on the data, HUVECs were treated with 0, 30, 50, and 100 nM of tunicamycin, and no significant effects on tube formation was noted (FIG. 6P). Thus, localization of HK1 plays a significant role in the regulation of protein N-glycosylation, but this process is not responsible for the defect in the angiogenic potential of ECs when HK1 is not bound to the mitochondria.

### Example 7—Increased O-GlcNAcylation in ECs Causes a Defect in Angiogenesis

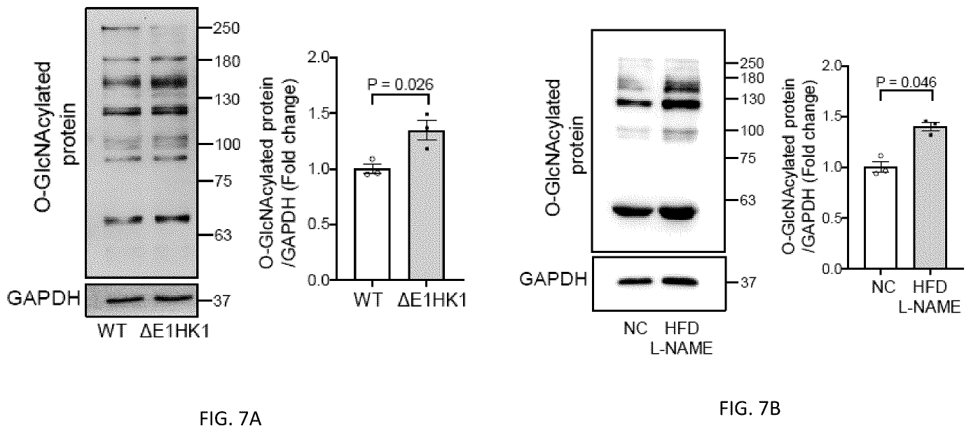

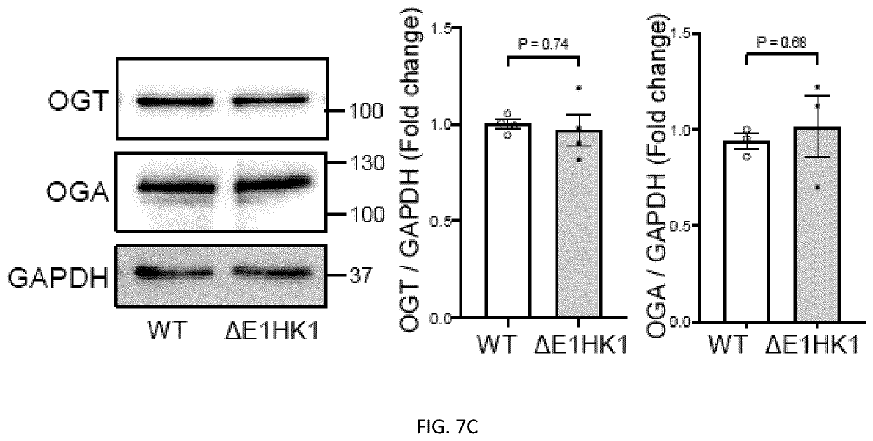

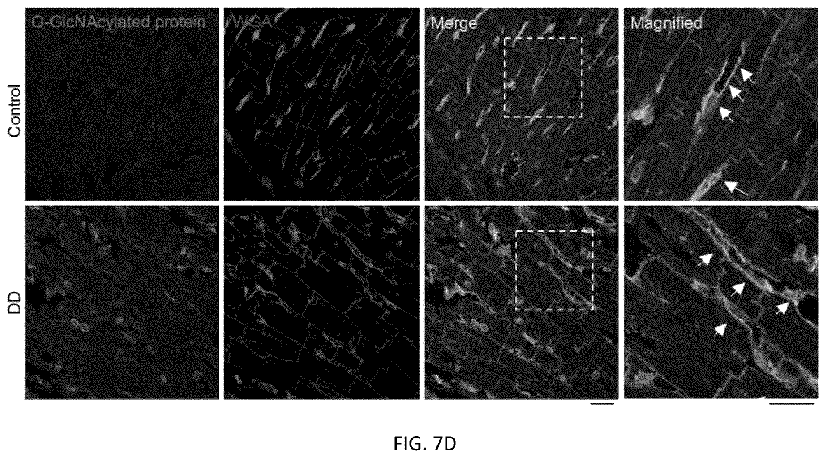

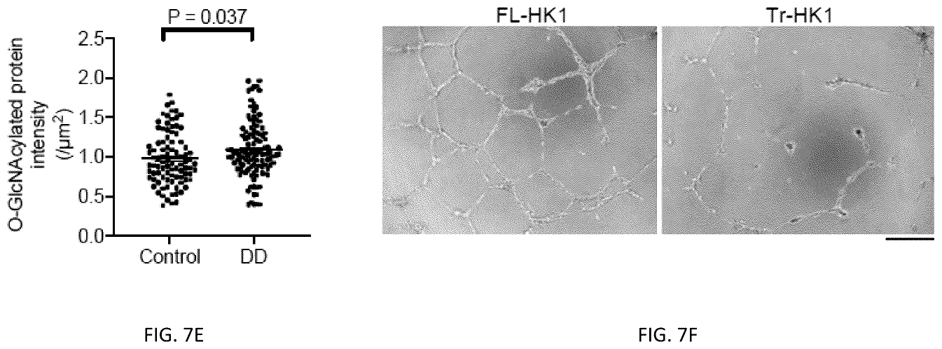

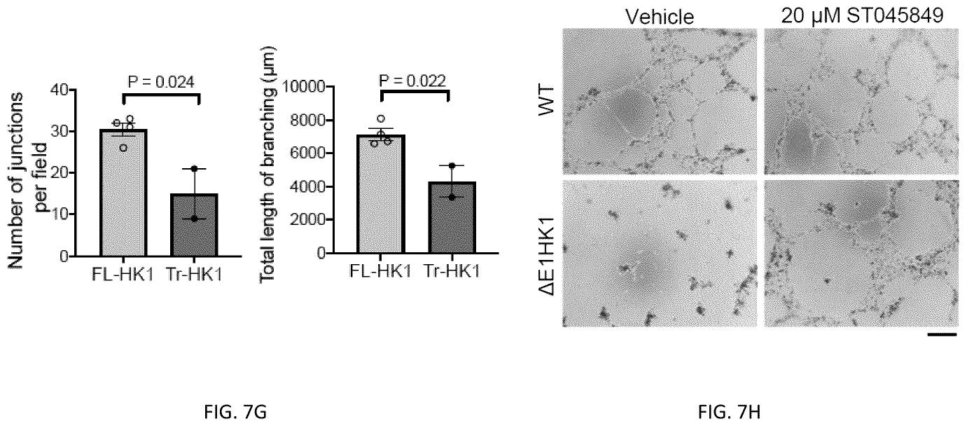

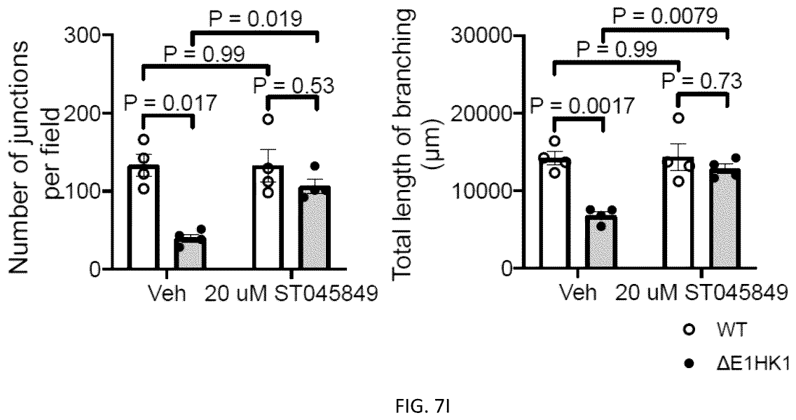

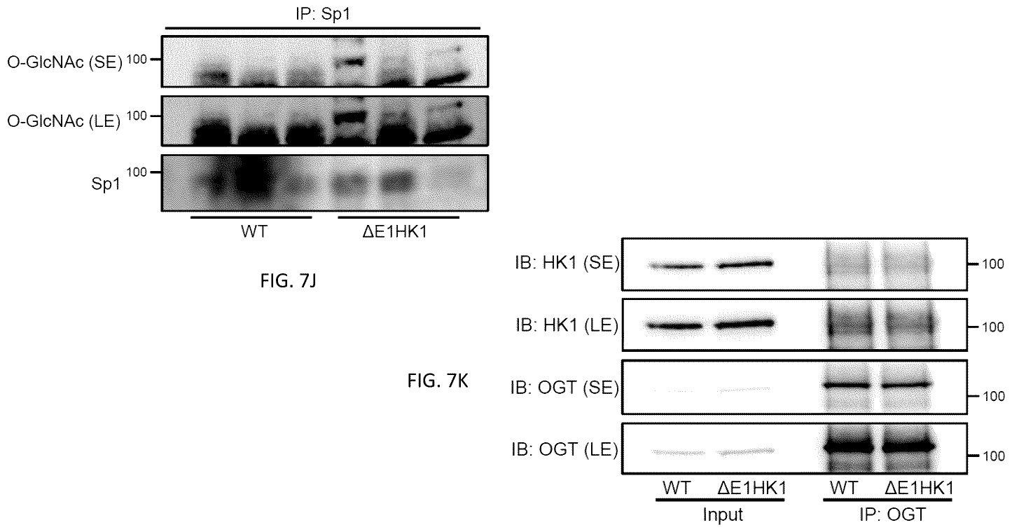

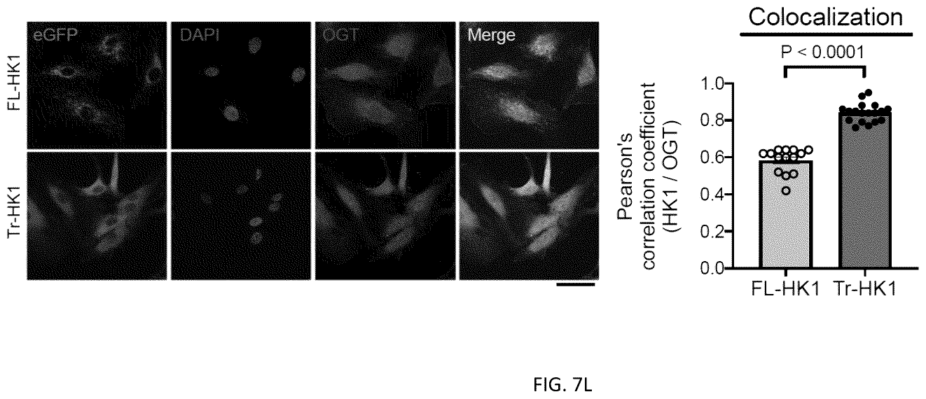

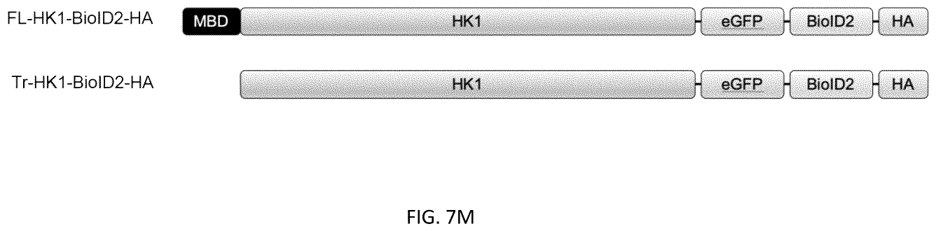

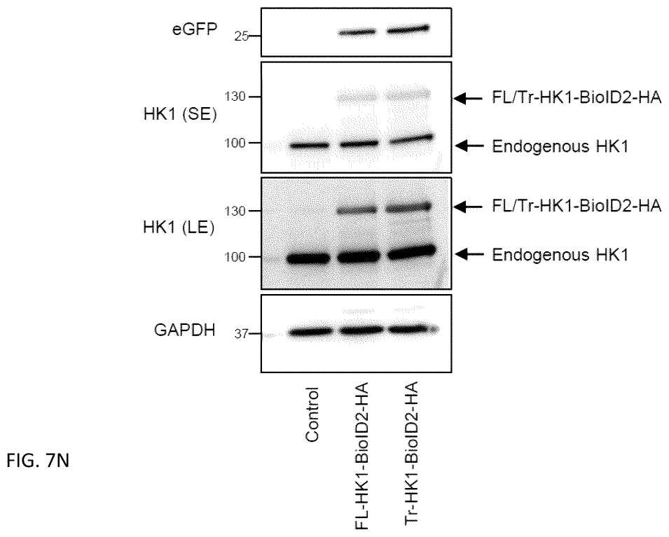

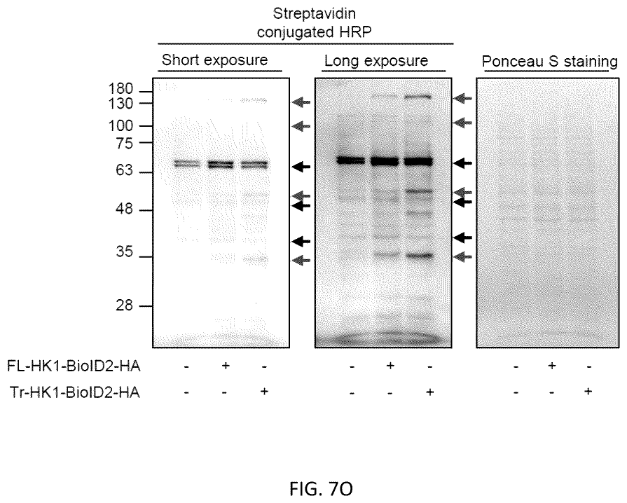

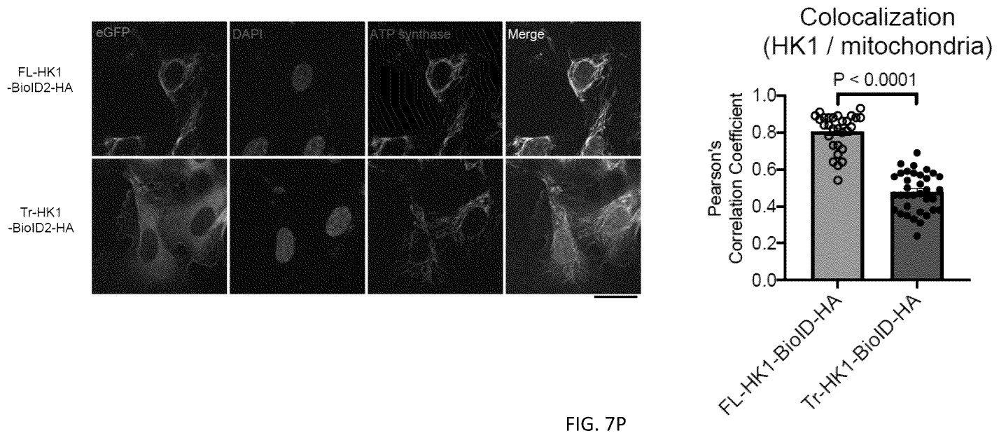

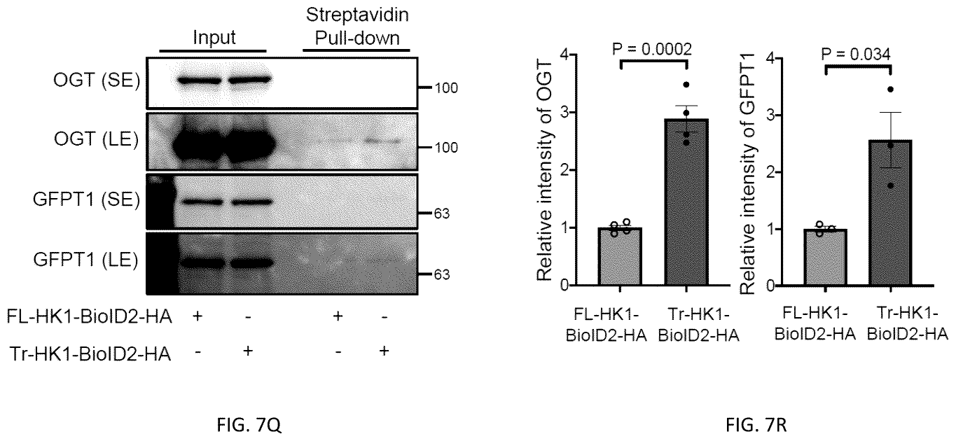

As the data indicated that the reduction in N-glycosylation was not the underlying cause of the angiogenic defect, the investigation shifted focus to another prominent outcome of the HBP: protein O-GlcNAcylation. This process is increased in diabetes and hypertension, which are both risk factors for the development of HFpEF (Lima et al., 2010; Makino et al., 2015). Immunoblots employing an anti-O-GlcNAcylated protein antibody on total cell lysates revealed an increased level of protein O-GlcNAcylation in ECs derived from ΔE1HK1 mice compared to WT mice (FIG. 7A). A similar increase in protein O-GlcNAcylation was observed in isolated ECs from C57BL/6J mice subjected to HFD_L-NAME treatment (FIG. 7B). Subsequently, the protein levels of two key enzymes in the O-GlcNAcylation machinery, OGT and OGA was quantified, and no significant differences was found between ECs from WT and ΔE1HK1 hearts (FIG. 7C). To further investigate O-GlcNAcylation in ECs, immunofluorescent staining on human tissues was conducted. The clinical features of the subjects are summarized in Supplemental Table 1, and significant increase in LV mass was highlighted in a group with LV diastolic dysfunction (DD) in comparison to without DD. The findings demonstrated an increased O-GlcNAcylation signal associated with capillaries in hearts exhibiting DD compared to those without DD (FIGS. 7D and 7E). To explore whether the heightened O-GlcNAcylation resulted from the gain-of-function of AE1HK1, FL-HK1 and Tr-HK1 were overexpressed in HUVECs. Notably, cells overexpressing Tr-HK1 displayed reduced tube formation in comparison to those overexpressing FL-HK1 (FIGS. 7F and 7G). Next, whether the increase in protein O-GlcNAcylation in ECs from the ΔE1HK1 mice was the causative factor for the diminished angiogenesis noted in these cells was studied. ECs from ΔE1HK1 mice were treated with the OGT inhibitor ST045849, resulting in a significant enhancement of tube formation markers, including the number of junctions and total length of branching (FIGS. 7H and 7I). Previous reports indicate the involvement of heightened Sp1 O-GlcNAcylation in endothelial cell dysfunction (Du et al., 2000). To assess the role of Sp1 O-GlcNAcylation in the development of HFpEF, immunoprecipitation studies were performed in ECs from WT and ΔE1HK1 mice, which showed increased O-GlcNAcylation of Sp1 in ΔE1HK1 ECs (FIG. 7J). These findings collectively indicate that the mechanism responsible for the reduced angiogenesis in the ECs from ΔE1HK1 mice is linked to an elevation in protein O-GlcNAcylation within the cells.

The machinery of O-GlcNAcylation predominantly resides within the cytosol and nucleus (Yang and Qian, 2017), a distinct spatial compartment from the N-glycosylation system. Considering that dislocated HK1 localizes to the cytosol and nucleus (FIGS. 2J-2O, FIGS. 4B and 4C and FIGS. 6G and 611), the inventors hypothesized that HBP intermediates undergo a redirection, shifting from protein N-glycosylation toward protein O-GlcNAcylation through HK1 mitochondrial dislocation. While direct binding of OGT and HK1 lacking the MBD (FIG. 7K) was not observed, immunofluorescent imaging demonstrated an enhanced colocalization between Tr-HK1 and OGT (FIG. 7L). Additionally, HUVECs that stably overexpressed either FL-HIK1 or Tr-HK1 were generated, each conjugated with BioID2 (Kim et al., 2016) (FIGS. 7M-7P), enabling investigation of the molecular-level proximity of HK1 lacking the MBD to enzymes related to 0-GlcNAcylation machinery. By administering biotin to these cells for 18 hours and subsequently purifying protein samples, distinct band patterns of biotinylated proteins between FL-HK1- and TrHK1-BioID2-HA were identified, confirming that these two proteins biotinylated different protein populations (FIG. 7O). Further analysis involved the isolation of biotinylated proteins using streptavidin-conjugated sepharose beads, which demonstrated a notably higher degree of biotinylation of OGT in HUVECs overexpressing Tr-HK1-BioID2 than FL-HK1 (FIGS. 7Q and 7R). Together, these data indicate the proximity of HK1 to OGT when it is dislocated from mitochondria, and this spatial proximity exerts a significant influence on modulating the balance of HBP intermediate allocation between the O-GlcNAcylation machinery and N-glycosylation.

### Example 8—Overexpression of OGA Reverses the HFpEF Phenotype

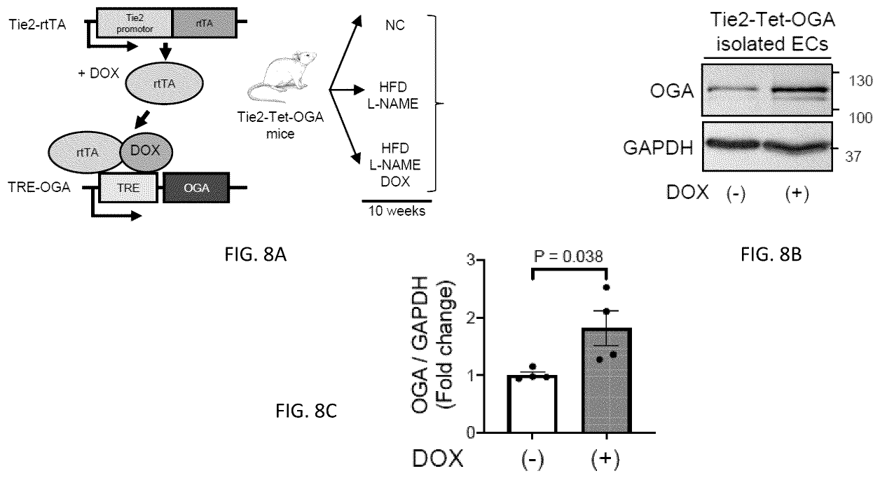

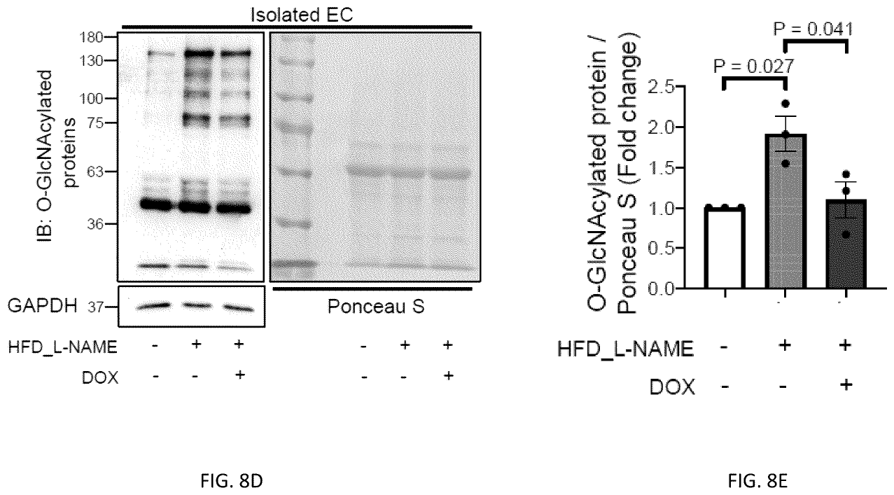

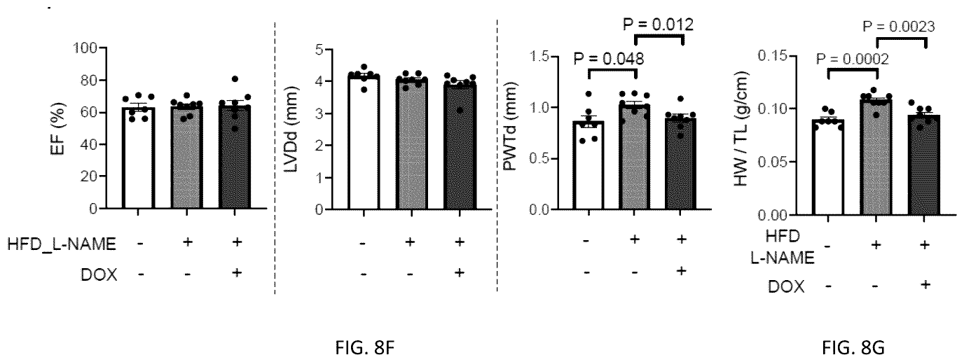

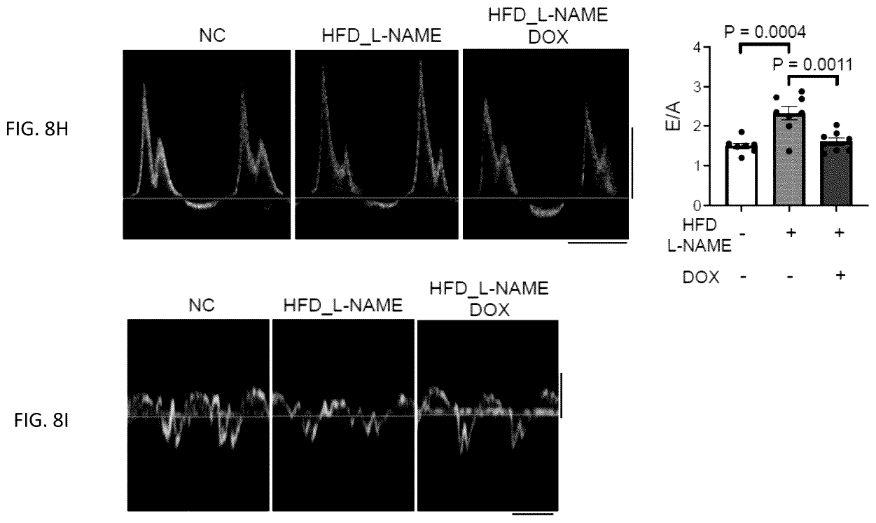

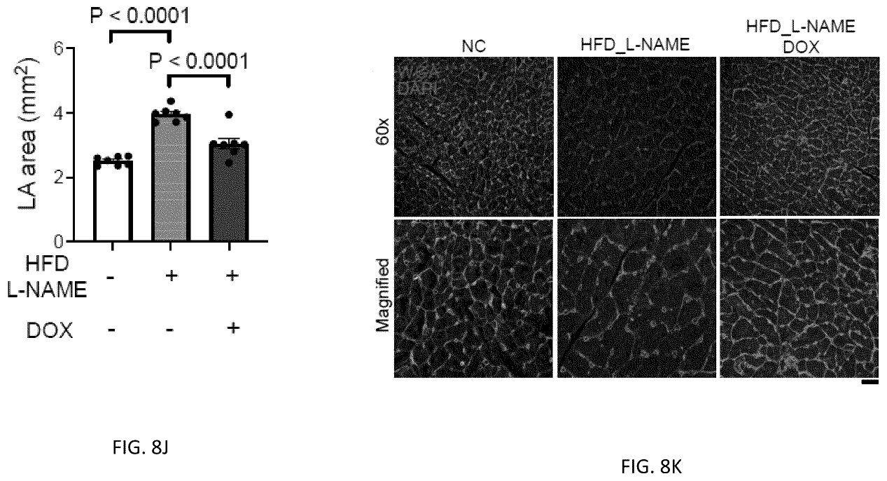

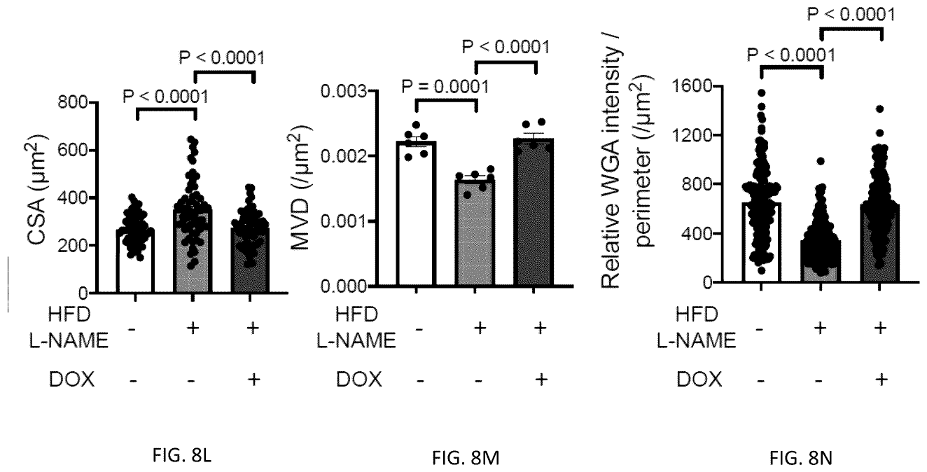

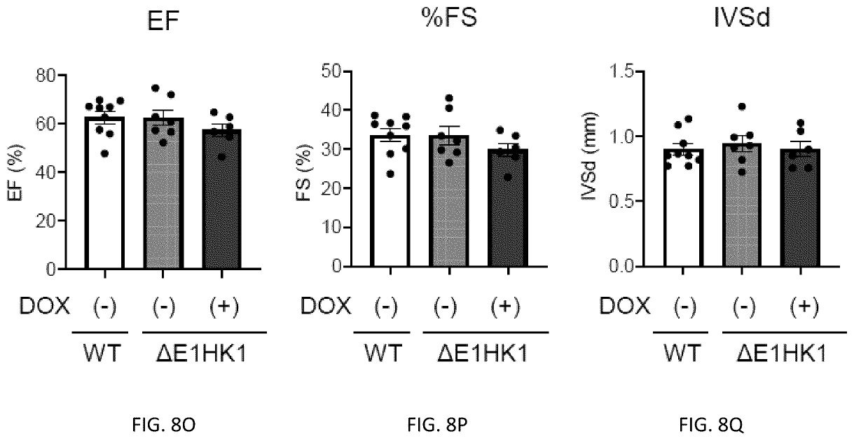

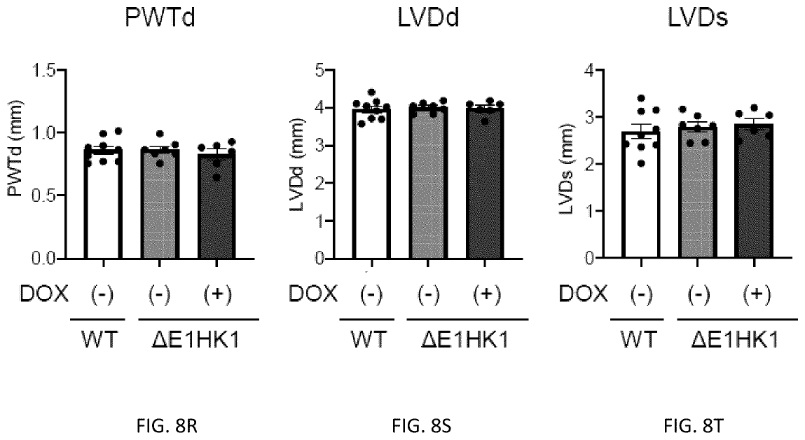

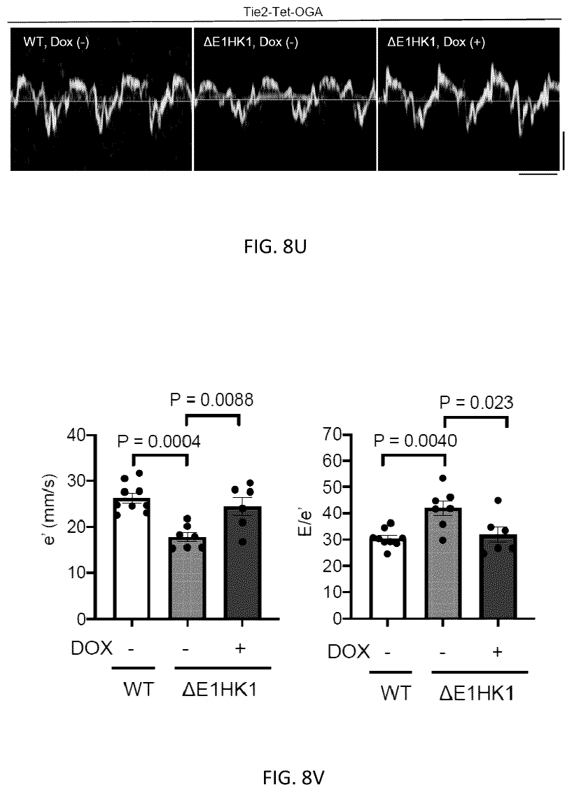

ΔE1HK1 mice have deletion of the MBD globally, so it may be argued that the HFpEF phenotype of these mice could be due to HK1 mitochondrial dislocation in other cells, besides the ECs. To address this concern, whether a reduction in protein O-GlcNAcylation specifically within ECs can reverse the HFpEF phenotype was studied. A doxycycline (DOX)-inducible, endothelial-specific OGA overexpression mouse model (Tie2-Tet-OGA) (Makino et al., 2015) was used, and the mice were subjected to various experimental conditions, including NC, HFD_L-NAME, or HFD_L-NAME combined with DOX induction to trigger OGA overexpression (FIG. 8A). The efficacy of OGA overexpression in ECs following DOX treatment was validated through western blot analysis (FIGS. 8B and 8C). Subsequent analyses revealed a reduction in total protein 0-GlcNAcylation within the ECs of DOX-treated mice (FIGS. 8D and 8E). These mice exhibited no alterations in EF, LV diameter in diastole (LVDd), but rather a significant reversal in posterior wall thickness in diastole (PWTd) (FIG. 8F) and heart weight (HW) (FIG. 8G). Furthermore, OGA overexpression in the ECs resulted in a reversal of HFpEF markers, including E/A, e', and LA area (FIGS. 811-8J). To further corroborate these findings, WGA fluorescent images of hearts confirmed the reversal of CM hypertrophy and microvascular rarefaction induced by HFD_LNAME following DOX administration (FIGS. 8K-8M). A reversal of the WGA signal intensity on capillaries with OGA overexpression was also observed (FIG. 8N). This observation suggested that the reduction in protein O-GlcNAcylation is sufficient to preserve protein N-glycosylation and maintain a balanced utilization of UDP-GlcNAc between those glycosylation machinery components. ΔE1HK1 was also crossed with Tie2-Tet-OGA mice. While no significant change was noted in EF and structural parameters (FIGS. 8O-8T), reduced e' in ΔE1HK1 mice was significantly reversed by EC specific OGA induction (FIGS. 8U and 8V). These collective results underscore that the reversal of protein O-GlcNAcylation within ECs is sufficient to counteract the HFpEF phenotype in those mouse models. Additionally, these studies into ΔE1HK1 crossed with Tie2-Tet-OGA mice suggest the critical roles of protein O-GlcNAcylation, particularly within ECs, in the development of HFpEF, despite the global deletion of the MBD of HK1 in ΔE1HK1 mice.

### Example 9—Pharmacological Inhibition of OGT Reverses the HFpEF Phenotype

The studies thus far have used genetic models to reverse the HFpEF phenotype. Next, whether the HFpEF phenotype can be reversed with pharmacological agents that can inhibit OGT was studied. Among the array of OGT inhibitors, 5S-GlcNHex has shown a significant in vivo efficacy (Liu et al., 2018). Dose dependency studies were performed and showed that 20 mg/kg of 5S-GlcNHex initiated the reduction of protein O-GlcNAcylation and reached a plateau with 30 mg/kg of 5S-GlcNHex (FIG. 9A). These dosages had no impact on body weight and systolic BP (FIGS. 9B and 9C). 20 mg/kg and 30 mg/kg of 5S-GlcNHex was then administered to mice every 48 hours for 10 weeks following 5 weeks of HFD_L-NAME treatment (FIG. 9D). While no significant changes were observed in EF, fraction shortening (FS), LVDd, and LV diameter in systole (LVDs) among the groups (FIGS. 9E and 9F), the increased E/A ratio in mice with HFD_L-NAME was reversed by 20 and 30 mg/kg of 5S-GlcNHex (FIGS. 9G and 911). Similarly, the increased LA sizes and heart weights in these mice displayed a tendency to decrease with the treatments (FIGS. 91 and 9J).

Moreover, a treadmill test demonstrated a significant improvement in exercise capacity with 30 mg/kg of 5S-GlcNHex (FIG. 9K). A single intraperitoneal injection of 50 mg/kg of 5S-GlcNHex to C57BL6/J mice was also administered, which effectively curtailed protein 0-GlcNAcylation in various organs, in line with a previous report (Liu et al., 2018). The levels of protein O-GlcNAcylation experienced a discernable reduction over 48 hours, eventually returning to baseline after 72 hours (FIG. 9M). The body weights of mice receiving drug treatment every 48 hours for 5 weeks remained comparable to those of their untreated counterparts (FIG. 9N). Treatment with 50 mg/kg of 5S-GlcNHex for 3 weeks following 5 weeks of HFD_LNAME treatment indicated that while no discernable effects were observed on EF, cardiac hypertrophy, as indicated by PWTd and heart weights, was ameliorated with 5S-GlcNHex treatment (FIGS. 90-9S). Additionally, increased E/A, E/e' and LA area, and decreased e' in mice treated with HFD_L-NAME were substantially restored by 50 mg/kg of 5S-GlcNHex (FIG. 9T-9X), suggesting its promising therapeutic potential in suppressing the development of HFpEF. These results collectively suggest that 5S-GlcNHex may be used as a novel pharmacological approach for reversing the HFpEF phenotype, holding significant clinical implications for addressing this prevalent disorder.

In the disclosure above, a novel mouse model of HFpEF is reported and hexokinase-1 mitochondrial binding in endothelial cells is critical for protein O-GlcNAcylation and the development of HFpEF is shown. Increased mitochondrial dislocation of HK1 in ECs from HFpEF mice is demonstrated. Mice with deletion of the mitochondrial-binding-domain of HK1 spontaneously develop HFpEF, and their ECs display impaired angiogenic potential. Mitochondrial-bound HK1 associates with dolichyl-diphosphooligosaccharide-protein-glycosyltransferase (DDOST) and its mitochondrial dislocation decreases protein N-glycosylation.

The spatial proximity of dislocated HK1 and N-acetylglucosamine transferase (OGT) increases protein O-GlcNAcylation by shifting the balance of the hexosamine-biosynthetic-pathway intermediate supply into the O-GlcNAcylation machinery is also shown. EC-specific overexpression of OGA reverses angiogenic defects in ECs and the HFpEF phenotype, indicating that increased protein O-GlcNAcylation in ECs is responsible for the development of HFpEF. Finally, it is shown that a novel OGT inhibitor reverses HFpEF phenotype, providing a possible new treatment for this devastating disorder. This disclosure demonstrates a new mechanism for HFpEF through HK1 cellular localization and resultant protein O-GlcNAcylation in ECs and provides a potential novel therapy for HFpEF.

In this description it will be readily apparent to one skilled in the art that varying substitutions and modifications may be made to the invention disclosed herein without departing from the scope and spirit of the invention. The invention illustratively described suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made throughout this disclosure. The cited references are incorporated by reference in their entireties. In the event that there is an inconsistency between a definition of a term in this specification as compared to a definition of the term in a cited reference, the term should be interpreted based on the definition in the specification.

### REFERENCES

Abel, E. D., Kaulbach, H. C., Tian, R., Hopkins, J. C., Duffy, J., Doetschman, T., Minnemann, T., Boers, M. E., Hadro, E., Oberste-Berghaus, C., et al. (1999). Cardiac hypertrophy with preserved contractile function after selective deletion of GLUT4 from the heart. J Clin Invest 104, 1703-1714.
Ackers-Johnson, M., Li, P. Y, Holmes, A. P., O'Brien, S. M., Pavlovic, D., and Foo, R. S. (2016). A Simplified, Langendorff-Free Method for Concomitant Isolation of Viable Cardiac Myocytes and Nonmyocytes From the Adult Mouse Heart. Circulation research 119, 909-920.
Adeva-Andany, M. M., Perez-Felpete, N., Fernandez-Fernandez, C., Donapetry-Garcia, C., and Pazos-Garcia, C. (2016). Liver glucose metabolism in humans. Biosci Rep 36.
Akella, N. M., Ciraku, L., and Reginato, M. J. (2019). Fueling the fire: emerging role of the hexosamine biosynthetic pathway in cancer. BMC Biol 17, 52.
Baghirova, S., Hughes, B. G., Hendzel, M. J., and Schulz, R. (2015). Sequential fractionation and isolation of subcellular proteins from tissue or cultured cells. MethodsX 2, 440-445.
Bell, G. I., Burant, C. F., Takeda, J., and Gould, G. W. (1993). Structure and function of mammalian facilitative sugar transporters. J Biol Chem 268, 19161-19164.
Bhatia, R. S., Tu, J. V., Lee, D. S., Austin, P. C., Fang, J., Haouzi, A., Gong, Y, and Liu, P. P. (2006). Outcome of heart failure with preserved ejection fraction in a population-based study. N Engl J Med 355, 260-269.
Borlaug, B. A., Olson, T. P., Lam, C. S., Flood, K. S., Lerman, A., Johnson, B. D., and Redfield, M. M. (2010). Global cardiovascular reserve dysfunction in heart failure with preserved ejection fraction. JAm Coll Cardiol 56, 845-854.
Chatham, J. C., Zhang, J., and Wende, A. R. (2021). Role of O-Linked N-Acetylglucosamine Protein Modification in Cellular (Patho)Physiology. Physiol Rev 101, 427-493.
Chen, C. Y, Caporizzo, M. A., Bedi, K., Vite, A., Bogush, A. I., Robison, P., Heffler, J. G., Salomon, A. K., Kelly, N. A., Babu, A., et al. (2018). Suppression of detyrosinated microtubules improves cardiomyocyte function in human heart failure. Nat Med 24, 1225-1233.
Cui, Y, Zheng, Y, Liu, X., Yan, L., Fan, X., Yong, J., Hu, Y, Dong, J., Li, Q., Wu, X., et al. (2019). Single-Cell Transcriptome Analysis Maps the Developmental Track of the Human Heart. Cell reports 26, 1934-1950 e1935.
Das, A., Huang, G. X., Bonkowski, M. S., Longchamp, A., Li, C., Schultz, M. B., Kim, L. J., Osborne, B., Joshi, S., Lu, Y, et al. (2018). Impairment of an Endothelial NAD(+)-H2S Signaling Network Is a Reversible Cause of Vascular Aging. Cell 173, 74-89 e20.
De Bock, K., Georgiadou, M., Schoors, S., Kuchnio, A., Wong, B. W., Cantelmo, A. R., Quaegebeur, A., Ghesquiere, B., Cauwenberghs, S., Eelen, G., et al. (2013). Role of PFKFB3-driven glycolysis in vessel sprouting. Cell 154, 651-663.
De Jesus, A., Keyhani-Nejad, F., Pusec, C. M., Goodman, L., Geier, J. A., Stoolman, J. S., Stanczyk, P. J., Nguyen, T., Xu, K., Suresh, K. V., et al. (2022). Hexokinase 1 cellular localization regulates the metabolic fate of glucose. Molecular cell 82, 1261-1277 e1269.
Dehennaut, V., Leprince, D., and Lefebvre, T. (2014). O-GlcNAcylation, an Epigenetic Mark. Focus on the Histone Code, TET Family Proteins, and Polycomb Group Proteins. Front Endocrinol (Lausanne) 5, 155.
Deng, Y, Xie, M., Li, Q., Xu, X., Ou, W., Zhang, Y, Xiao, H., Yu, H., Zheng, Y, Liang, Y, et al. (2021). Targeting Mitochondria-Inflammation Circuit by beta-Hydroxybutyrate Mitigates HFpEF. Circ Res 128, 232-245.
Dhingra, A., Garg, A., Kaur, S., Chopra, S., Batra, J. S., Pandey, A., Chaanine, A. H., and Agarwal, S. K. (2014). Epidemiology of heart failure with preserved ejection fraction. Curr Heart Fail Rep 11, 354-365.
Du, X. L., Edelstein, D., Dimmeler, S., Ju, Q., Sui, C., and Brownlee, M. (2001). Hyperglycemia inhibits endothelial nitric oxide synthase activity by posttranslational modification at the Akt site. Journal of Clinical Investigation 108, 1341-1348.
Du, X. L., Edelstein, D., Rossetti, L., Fantus, I. G., Goldberg, H., Ziyadeh, F., Wu, J., and Brownlee, M. (2000). Hyperglycemia-induced mitochondrial superoxide overproduction activates the hexosamine pathway and induces plasminogen activator inhibitor-1 expression by increasing Sp1 glycosylation. Proceedings of the National Academy of Sciences of the United States of America 97, 12222-12226.
Farrero, M., Blanco, I., Batlle, M., Santiago, E., Cardona, M., Vidal, B., Castel, M. A., Sitges, M., Barbera, J. A., and Perez-Villa, F. (2014). Pulmonary hypertension is related to peripheral endothelial dysfunction in heart failure with preserved ejection fraction. Circ Heart Fail 7, 791-798.
Federici, M., Menghini, R., Mauriello, A., Hribal, M. L., Ferrelli, F., Lauro, D., Sbraccia, P., Spagnoli, L. G., Sesti, G., and Lauro, R. (2002). Insulin-dependent activation of endothelial nitric oxide synthase is impaired by O-linked glycosylation modification of signaling proteins in human coronary endothelial cells. Circulation 106, 466-472.
Fonarow, G. C., Stough, W. G., Abraham, W. T., Albert, N. M., Gheorghiade, M., Greenberg, B. H., O'Connor, C. M., Sun, J. L., Yancy, C. W., Young, J. B., et al. (2007). Characteristics, treatments, and outcomes of patients with preserved systolic function hospitalized for heart failure: a report from the OPTIMIZE-HF Registry. J Am Coll Cardiol 50, 768-777.
Gould, G. W., and Holman, G. D. (1993). The glucose transporter family: structure, function and tissue-specific expression. Biochem J 295 (Pt 2), 329-341.
Gussak, G., Marszalec, W., Yoo, S., Modi, R., O'Callaghan, C., Aistrup, G. L., Cordeiro, J. M., Goodrow, R., Kanaporis, G., Blatter, L. A., et al. (2020). Triggered Ca(2+) Waves Induce Depolarization of Maximum Diastolic Potential and Action Potential Prolongation in Dog Atrial Myocytes. Circ Arrhythm Electrophysiol 13, e008179.
Haass, M., Kitzman, D. W., Anand, I. S., Miller, A., Zile, M. R., Massie, B. M., and Carson, P. E. (2011). Body mass index and adverse cardiovascular outcomes in heart failure patients with preserved ejection fraction: results from the Irbesartan in Heart Failure with Preserved Ejection Fraction (I-PRESERVE) trial. Circ Heart Fail 4, 324-331.
Hanover, J. A., Krause, M. W., and Love, D. C. (2012). Bittersweet memories: linking metabolism to epigenetics through O-GlcNAcylation. Nat Rev Mol Cell Biol 13, 312-321.
Jin, S. P., and Chung, J. H. (2018). Inhibition of N-glycosylation by tunicamycin attenuates cell-cell adhesion via impaired desmosome formation in normal human epidermal keratinocytes. Biosci Rep 38.
Ju Kim, E. (2020). O-GlcNAc Transferase: Structural Characteristics, Catalytic Mechanism and Small-Molecule Inhibitors. ChemBioChem 21, 3026-3035.
Justus, C. R., Leffler, N., Ruiz-Echevarria, M., and Yang, L. V. (2014). In vitro cell migration and invasion assays. J Vis Exp.
Kardys, I., Deckers, J. W., Stricker, B. H., Vletter, W. B., Hofman, A., and Witteman, J. C. (2009). Echocardiographic parameters and all-cause mortality: the Rotterdam Study. Int J Cardiol 133, 198-204.
Kim, D. I., Jensen, S. C., Noble, K. A., Kc, B., Roux, K. H., Motamedchaboki, K., and Roux, K. J. (2016). An improved smaller biotin ligase for BiolD proximity labeling. Mol Biol Cell 27, 1188-1196.
Lewis, B. A., and Hanover, J. A. (2014). O-GlcNAc and the epigenetic regulation of gene expression. J Biol Chem 289, 34440-34448.
Li, X., Sun, X., and Carmeliet, P. (2019). Hallmarks of Endothelial Cell Metabolism in Health and Disease. Cell Metab 30, 414-433.
Lima, VV., Giachini, F. R., Carneiro, F. S., Carneiro, Z. N., Saleh, M. A., Pollock, D. M., Fortes, Z. B., Carvalho, M. H., Ergul, A., Webb, R. C., and Tostes, R. C. (2010). O-GlcNAcylation contributes to augmented vascular reactivity induced by endothelin 1. Hypertension 55, 180-188.
Liu, T. W., Zandberg, W. F., Gloster, T. M., Deng, L., Murray, K. D., Shan, X., and Vocadlo, D. J. (2018). Metabolic Inhibitors of O-GlcNAc Transferase That Act In Vivo Implicate Decreased O-GlcNAc Levels in Leptin-Mediated Nutrient Sensing. Angew Chem Int Ed Engl 57, 7644-7648.
Luo, B., Soesanto, Y, and McClain, D. A. (2008). Protein modification by O-linked GlcNAc reduces angiogenesis by inhibiting Akt activity in endothelial cells. Arterioscler Thromb Vasc Biol 28, 651-657.
Luo, S., Truong, A. H., and Makino, A. (2016). Isolation of Mouse Coronary Endothelial Cells. J Vis Exp.
Makino, A., Dai, A., Han, Y, Youssef, K. D., Wang, W., Donthamsetty, R., Scott, B. T., Wang, H., and Dillmann, W. H. (2015). O-GlcNAcase overexpression reverses coronary endothelial cell dysfunction in type 1 diabetic mice. Am J Physiol Cell Physiol 309, C593-599.
Middleton, R. J. (1990). Hexokinases and glucokinases. Biochem Soc Trans 18, 180-183.
Mohammed, S. F., Hussain, S., Mirzoyev, S. A., Edwards, W. D., Maleszewski, J. J., and Redfield, M. M. (2015). Coronary microvascular rarefaction and myocardial fibrosis in heart failure with preserved ejection fraction. Circulation 131, 550-559.
Mueckler, M. (1994). Facilitative glucose transporters. Eur J Biochem 219, 713-725.
Nacev, B. A., Grassi, P., Dell, A., Haslam, S. M., and Liu, J. O. (2011). The antifungal drug itraconazole inhibits vascular endothelial growth factor receptor 2 (VEGFR2) glycosylation, trafficking, and signaling in endothelial cells. J Biol Chem 286, 44045-44056.
Nagaraj, R., Sharpley, M. S., Chi, F., Braas, D., Zhou, Y, Kim, R., Clark, A. T., and Banerjee, U. (2017). Nuclear Localization of Mitochondrial TCA Cycle Enzymes as a Critical Step in Mammalian Zygotic Genome Activation. Cell 168, 210-223 e211.
Pastorino, J. G., Shulga, N., and Hoek, J. B. (2002). Mitochondrial binding of hexokinase II inhibits Bax-induced cytochrome c release and apoptosis. J Biol Chem 277, 7610-7618.
Paulus, W. J., and Tschope, C. (2013). A novel paradigm for heart failure with preserved ejection fraction: comorbidities drive myocardial dysfunction and remodeling through coronary microvascular endothelial inflammation. J Am Coll Cardiol 62, 263-271.
Printz, R. L., Osawa, H., Ardehali, H., Koch, S., and Granner, D. K. (1997). Hexokinase II gene: structure, regulation and promoter organization. Biochem Soc Trans 25, 107-112.
Rieusset, J. (2018). The role of endoplasmic reticulum-mitochondria contact sites in the control of glucose homeostasis: an update. Cell Death Dis 9, 388.
Rizzuto, R., De Stefani, D., Raffaello, A., and Mammucari, C. (2012). Mitochondria as sensors and regulators of calcium signalling. Nat Rev Mol Cell Biol 13, 566-578.
Rossi, A., Rigotto, G., Valente, G., Giorgio, V., Basso, E., Filadi, R., and Pizzo, P. (2020). Defective Mitochondrial Pyruvate Flux Affects Cell Bioenergetics in Alzheimer's Disease-Related Models. Cell Rep 30, 2332-2348 e2310.
Schiattarella, G. G., Altamirano, F., Tong, D., French, K. M., Villalobos, E., Kim, S. Y, Luo, X., Jiang, N., May, H. I., Wang, Z. V., et al. (2019). Nitrosative stress drives heart failure with preserved ejection fraction. Nature 568, 351-356.
Sears, R. M., May, D. G., and Roux, K. J. (2019). BiolD as a Tool for Protein-Proximity Labeling in Living Cells. Methods Mol Biol 2012, 299-313.
Shah, S. J., Lam, C. S. P., Svedlund, S., Saraste, A., Hage, C., Tan, R. S., Beussink-Nelson, L., Ljung Faxen, U., Fermer, M. L., Broberg, M. A., et al. (2018). Prevalence and correlates of coronary microvascular dysfunction in heart failure with preserved ejection fraction: PROMIS-HFpEF. Eur Heart J 39, 3439-3450.
Singh, J. P., Zhang, K., Wu, J., and Yang, X. (2015). O-GlcNAc signaling in cancer metabolism and epigenetics. Cancer Lett 356, 244-250.
Sui, D., and Wilson, J. E. (1997). Structural determinants for the intracellular localization of the isozymes of mammalian hexokinase: intracellular localization of fusion constructs incorporating structural elements from the hexokinase isozymes and the green fluorescent protein. Arch Biochem Biophys 345, 111-125.
Sun, L., Shukair, S., Naik, T. J., Moazed, F., and Ardehali, H. (2008). Glucose phosphorylation and mitochondrial binding are required for the protective effects of hexokinases I and II. Molecular and cellular biology 28, 1007-1017.
Szymanski, J., Janikiewicz, J., Michalska, B., Patalas-Krawczyk, P., Perrone, M., Ziolkowski, W., Duszynski, J., Pinton, P., Dobrzyn, A., and Wieckowski, M. R. (2017). Interaction of Mitochondria with the Endoplasmic Reticulum and Plasma Membrane in Calcium Homeostasis, Lipid Trafficking and Mitochondrial Structure. Int J Mol Sci 18.
Taqueti, V. R., Solomon, S. D., Shah, A. M., Desai, A. S., Groarke, J. D., Osborne, M. T., Hainer, J., Bibbo, C. F., Dorbala, S., Blankstein, R., and Di Carli, M. F. (2018). Coronary microvascular dysfunction and future risk of heart failure with preserved ejection fraction. Eur Heart J 39, 840-849.
Ureta, T. (1982). The comparative isozymology of vertebrate hexokinases. Comp Biochem Physiol B 71, 549-555.
van Beijnum, J. R., Rousch, M., Castermans, K., van der Linden, E., and Griffioen, A. W. (2008). Isolation of endothelial cells from fresh tissues. Nat Protoc 3, 1085-1091.
Vite, A., Caporizzo, M. A., Corbin, E. A., Brandimarto, J., McAfee, Q., Livingston, C. E., Prosser, B. L., and Margulies, K. B. (2022). Extracellular stiffness induces contractile dysfunction in adult cardiomyocytes via cell-autonomous and microtubule-dependent mechanisms. Basic Res Cardiol 117, 41.
Wilson, J. E. (2003). Isozymes of mammalian hexokinase: structure, subcellular localization and metabolic function. J Exp Biol 206, 2049-2057.
Withaar, C., Lam, C. S. P., Schiattarella, G. G., de Boer, R. A., and Meems, L. M. G. (2021a). Heart failure with preserved ejection fraction in humans and mice: embracing clinical complexity in mouse models. Eur Heart J 42, 4420-4430.
Withaar, C., Meems, L. M. G., Markousis-Mavrogenis, G., Boogerd, C. J., Sillje, H. H. W., Schouten, E. M., Dokter, M. M., Voors, A. A., Westenbrink, B. D., Lam, C. S. P., and de Boer, R. A. (2021b). The effects of liraglutide and dapagliflozin on cardiac function and structure in a multi-hit mouse model of heart failure with preserved ejection fraction. Cardiovasc Res 117, 2108-2124.
Xu, B., and Daimon, M. (2016). Cardiac aging phenomenon and its clinical features by echocardiography. Journal of echocardiography 14, 139-145.
Yang, X., and Qian, K. (2017). Protein O-GlcNAcylation: emerging mechanisms and functions. Nat Rev Mol Cell Biol 18, 452-465.

We claim:

1. A method of treating or preventing heart failure with preserved ejection fraction (HFpEF) comprising administering a composition comprising an O-linked N-acetylglucosamine transferase (OGT) inhibitor, wherein the composition treats the HFpEF.

2. The method of claim 1, wherein the OGT inhibitor is 2-deoxy-2-N-hexanamide-5-thioD-glucopyranoside (5SGlcNHex).

3. The method of claim 2, wherein the composition is a pharmaceutical composition, and wherein the pharmaceutical composition is administered to a subject with or at risk of experiencing HFpEF.

4. The method of claim 1, wherein the OGT inhibitor reduces protein O-linked N-acetylglucosaminylation (O-GlcNAcylation) in endothelial cells of the heart.

5. The method of claim 4, wherein the protein O-GlcNAcylation in the endothelial cells is mediated by hexokinase-1 (HK-1) dislocation from mitochondria in the cells.

6. The method of claim 3, wherein diastolic function is restored or maintained in the heart of the subject.

7. The method of claim 1, wherein OGT inhibitor restores or maintains angiogenic function in endothelial cells of the heart.

8. A method of treating or preventing heart failure with preserved ejection fraction (HFpEF) comprising administering a composition that increases O-linked N-acetylglucosaminase (OGA) in endothelial cells of the heart.

9. The method of claim 8, wherein the composition protects against angiogenic defect in the endothelial cells.

10. The method of claim 9, wherein the angiogenic defect is associated with increased protein O-linked N-acetylglucosaminylation (O-GlcNAcylation) in the endothelial cells.

11. The method of claim 8, wherein the method reduces O-GlcNAcylation in the endothelial cells.

12. The method of claim 8, wherein diastolic function is maintained in the heart of a subject who is at risk of developing HFpEF.

13. The method of claim 8, wherein diastolic function is restored in the heart of a subject who has experienced HFpEF.

14. The method of claim 8, wherein cardiomyocyte hypertrophy in a subject who has experienced HFpEF is reduced or eliminated.

15. The method of claim 8, wherein macrovascular rarefaction in a subject who has experienced HFpEF is reduced or eliminated.
